(19) United States
(12) Patent Application Publication
(10) Pub. No.: US 2023/0266326 A1
(43) Pub. Date: Aug. 24, 2023

(54) HOST SIGNATURES FOR PREDICTING IMMUNOTHERAPY RESPONSE

(71) Applicant: ONCOHOST LTD., Binyamina (IL)

(72) Inventors: Coren LAHAV, Tel Aviv (IL); Michal HAREL, Hod Hasharon (IL); Eran ISSLER, Zichron Yaakov (IL)

(21) Appl. No.: 18/012,007

(22) PCT Filed: Jun. 21, 2021
(86) PCT No.: PCT/IL2021/050756

§ 371 Date: Dec. 21, 2022

Related U.S. Application Data
(60) Provisional application No. 63/041,971, filed on Jun. 21, 2020.

(51) Int. Cl. G01N 33/574

(52) U.S. Cl. CPC G01N 33/57423; G01N 33/57488; G01N 2800/52

(57) ABSTRACT

Methods of determining a therapeutic response to immunotherapy in a subject suffering from lung cancer are provided. Kits for use in determining a therapeutic response to immunotherapy are also provided.

### CROSS REFERENCE TO RELATED APPLICATIONS

This application claims the benefit of priority of U.S. Provisional Patent Application No. 63/041,971, Jun. 21, 2020, the contents of which are all incorporated herein by reference in their entirety.

### FIELD OF INVENTION

The present invention is in the field of immunotherapy response.

### BACKGROUND OF THE INVENTION

One of the major complications in oncology is resistance to therapy. Many studies have focused on the involvement of mutations and epigenetic changes in tumor cells in conferring drug resistance. However, in recent years, studies have indicated that in response to almost any type of anti-cancer therapy, the patient (i.e., the host) may generate pro-tumorigenic and pro-metastatic effects. This phenomenon, called host-response, is the physiological reaction of the patient to the cancer therapy that potentially counteracts the anti-tumor activity of the treatment.

Non-small cell lung cancer (NSCLC) is the leading cause of cancer-related deaths world-wide. Immunotherapeutic agents have become the most promising type of treatment in NSCLC. However, several limitations exist with these therapeutic agents when used as monotherapy, with objective responses observed in only 20-30% of patients. In addition, immune mechanisms involved in the response to these therapeutic interventions remain poorly elucidated. Thus, advanced proteomic technologies enabling an easy and non-invasive means for the discovery of blood-based protein biomarkers promise to identify host and tumor changes associated with immunotherapy response/non-response, and uncover biological mechanisms underlying host related primary resistance. A method of determining which subjects will respond to immunotherapy is greatly needed.

### SUMMARY OF THE INVENTION

The present invention provides methods for determining a therapeutic response to immunotherapy in a subject suffering from cancer, comprising determining expression levels of at least one factor in a biological sample obtained from the subject at a first time point relative to the immunotherapy, and/or determining expression levels of the at least one factor in a biological sample obtained from the subject at a second time point relative to the immunotherapy, wherein a differential expression level of one or more factors is indicative of the responsiveness of the subject to the immunotherapy. Kits for performance of a method of the invention are also provided.

According to a first aspect, there is provided a method for predicting a therapeutic response to immunotherapy in a subject suffering from lung cancer, the method comprising:
a) determining an expression level of at least two factors selected from Plasminogen activator, urokinase receptor (PLAUR), Cadherin 3 (CDH3), Interleukin 6 (IL6), Nectin cell adhesion molecule 1 (NECTIN1), Vascular endothelial growth factor D (VEGFD), Bone morphogenetic protein 4 (BMP4), X-linked inhibitor of apoptosis (XIAP), Interleukin 2 (IL2), Proline and arginine rich end leucine rich repeat protein (PRELP), Fibroblast growth factor 17 (FGF17), Mucin 16, cell surface associated (MUC16), Periostin (POSTN), MST1, Keratin 18 (KRT18), Ephrin A4 (EFNA4), Interleukin 4 receptor (IL4R), Granulysin (GNLY), Interleukin 18 (IL18), Beta-1,4-galactosyltransferase 1 (B4GALT1), Growth differentiation factor 2 (GDF2), Semaphorin 4D (SEMA4D), Erythropoietin receptor (EPOR), Ephrin type-B receptor 3 (EPHB3), Hepatocyte growth factor (HGF), Interferon alpha and beta receptor subunit 2 (IFNAR2), Secreted phosphoprotein 1 (SPP1), Fibronectin leucine rich transmembrane protein 1 (FLRT1), Inducible T cell costimulatory ligand (ICOSLG), Notch receptor 3 (NOTCH3), Neurturin (NRTN), Carbohydrate sulfotransferase 2 (CHST2), C-C motif chemokine ligand 1 (CCL1), Cluster of differentiation 97 (CD97), Linker for activation of T cells family member 2/non-T cell activation linker (LAT2), Dorsal inhibitory axon guidance protein (DRAXIN), Insulin like growth factor binding protein 4 (IGFBP4), TAFA Chemokine like family member 5 (TAFA5), Insulin like growth factor binding protein 5 (IGFBP-5), REST corepressor 1 (RCOR1), C-C motif chemokine ligand 11 (CCL11), Interleukin 12B (IL12B), Cystatin B (CSTB), Nucleobindin 2 (NUCB2), Pancreatic polypeptide (PPY) and DNA fragmentation factor subunit alpha (DFFA) in a biological sample obtained from the subject at a first time point relative to the immunotherapy;
b) determining an expression level of the at least two factors in a biological sample obtained from the subject at a second time point relative to the immunotherapy;
c) calculating a fold-change in expression of the at least two factors from the first time point to the second time point; and
d) analyzing the calculated fold-changes with a machine learning classifier, wherein the classifier is trained on a training set of fold-changes of the at least two factors in subjects who are known responders and non-responders, and wherein the classifier outputs a response prediction for the subject;
thereby predicting a therapeutic response to immunotherapy in a subject.

According to some embodiments, a response prediction comprises a response score, and wherein a response score below a predetermined threshold indicates the subject is a non-responder, and a response score above a predetermined threshold indicates the subject is a responder.

According to some embodiments, the first time point is a time point before the administration of the immunotherapy and the second time point is a time point after the administration of the immunotherapy.

According to another aspect, there is provided a method for predicting a therapeutic response to immunotherapy in a subject suffering from lung cancer, the method comprising:
a) determining an expression level of at least one factor selected from PLAUR, CDH3, IL6, NECTIN1, VEGFD, BMP4, XIAP, IL2, PRELP, FGF17, MUC16, POSTN, MST1, KRT18, EFNA4, IL4R, GNLY, IL18, B4GALT1, GDF2, SEMA4D, EPOR, EPHB3, HGF, IFNAR2, SPP1, FLRT1, ICOSGL NOTCH3, NRTN, CHST2, CCL1, CD97, LAT2, DRAXIN, IGFBP4, TAFA5, IGFBP5, RCOR1, CCL11, IL12B, CSTB, NUCB2, PPY and DFFA in a biological sample obtained from the subject before initiation of the immunotherapy; and
b) determining an expression level of the at least one factor in a biological sample obtained from the subject after initiation of immunotherapy;
wherein the factor is selected from BMP4, MUC16, KRT18, DRAXIN, EPHB3, EFNA4, GNLY, HGF, CCL1, IGFBP4, IL12B, IL18, IL2, IL4R, IL6, SPP1, CDH2, POSTN, SEMA4D, TAFA5, PLAUR and DFFA and an increased expression level of the at least one factor in the sample after initiation of immunotherapy is indicative of the subject being a non-responder to the immunotherapy, or wherein the factor is selected from B4GALT1, ICOSLG, BMP9, CCL11, CD97, CHST2, EPOR, FGF17, FLRT1, IFNAR2, IGFBP5, MST1, NECTIN1, NOTCH3, LAT2, NRTN, PRELP, RCOR1, VEGFD, CSTB, NUCB2, PPY and XIAP and an increased expression level of the at least one factor in the sample after initiation of immunotherapy is indicative of the subject being a responder to the immunotherapy, thereby predicting a therapeutic response to immunotherapy in a subject.

According to some embodiments, the factor is selected from B4GALT1, ICOSLG, BMP9, CCL11, CD97, CHST2, EPOR, FGF17, FLRT1, IFNAR2, IGFBP5, MST1, NECTIN1, NOTCH3, LAT2, NRTN, PRELP, RCOR1, VEGFD, CSTB, NUCB2, PPY and XIAP and a lack of increased expression level of the at least one factor in the sample after initiation of immunotherapy is indicative of the subject being a non-responder to the immunotherapy.

According to some embodiments, the at least one factor is selected from MUC16, IL6, ICOSLG, B4GALT1, CSTB, SPP1, CDH2, NUCB2, PPY, PLAUR, and DFFA.

According to some embodiments, determining an expression level comprises determining an expression level of a response signature, and wherein the response signature comprises at least a first factor selected from PLAUR, CDH3, and IL6, and at least a second factor selected from PLAUR, CDH3, IL6, BMP4, PRELP, SSP1, NRTN, NECTIN1, IL18, FGF17, CCL11, and IL12B.

According to some embodiments, the immunotherapy is immune checkpoint blockade.

According to some embodiments, the immune checkpoint blockade comprises an anti-PD-1/PD-L1 immunotherapy.

According to some embodiments, the lung cancer is non-small cell lung cancer (NSCLC).

According to some embodiments, the response signature is selected from the group consisting of:
a) PLAUR, CDH3, IL6, BMP4, PRELP and SPP1;
b) PLAUR, PRELP and IL6;
c) PLAUR and IL6;
d) CDH3, IL6, BMP4, PRELP and SPP1;
e) PLAUR and PRELP;
f) PLAUR, IL6, and BMP4;
g) CDH3 and PRELP;
h) IL6 and NRTN;
i) CDH3, IL18 and FGF17;
j) IL6 and NECTIN1;
k) CDH3 and NECTIN1;
l) PRELP and IL6; and
m) CDH3, CCL11 and IL12B.

According to some embodiments, the biological sample is plasma.

According to some embodiments, the expression level is a protein expression level or an mRNA expression level.

According to some embodiments, the expression level is a protein expression level.

According to some embodiments, the method comprises determining expression levels of a plurality of factors.

According to some embodiments, the increase is by at least a pre-determined threshold.

According to some embodiments, the method further comprises administering the immunotherapy between step (a) and step (b).

According to some embodiments, the method further comprises continuing to administer the immunotherapy to a subject who is not a non-responder.

According to some embodiments, the method further comprises administering to a non-responder an agent that modulates a pathway differentially regulated in the non-responder.

According to another aspect, there is provided a kit comprising reagents adapted to specifically determine the expression levels of at least two factors selected from PLAUR, CDH3, IL6, NECTIN1, VEGFD, BMP4, XIAP, IL2, PRELP, FGF17, MUC16, POSTN, MMST1SP, KRT18, EFNA4, IL4R, GNYL, IL18, B4GALT1, BMP9, SEMA4D, EPOR, EPHB3, HGF, IFNAR2, SPP1, FLRT1, ICOSLG, NOTCH3, CHST2, CCL1, CD97, LAT2, DRAXIN, IGFBP4, TAFA5, IGFBP5, RCOR1, NRTN, CCL11, IL12B, CSTB, NUCB2, PPY and DFFA and comprising at most 50 different reagents.

According to some embodiments, the at least two factors are selected from MUC16, IL6, ICOSLG, B4GALT1, CSTB, SPP1, CDH3, NUCB2, PPY, PLAUR, and DFFA.

According to some embodiments, the kit comprises reagents adapted to specifically determine the expression level of at least a first factor selected from PLAUR, CDH3, and IL6, and at least a second factor selected from PLAUR, CDH3, IL6, BMP4, PRELP, SPP1, NRTN, NECTIN1, and IL18+FGF17.

According to some embodiments, the kit comprises reagents adapted to specifically determine the expression level of at least one group of factors selected from the group consisting of:
n) PLAUR, CDH3, IL6, BMP4, PRELP and SPP1;
o) PLAUR, PRELP and IL6;
p) PLAUR and IL6;
q) CDH3, IL6, BMP4, PRELP and SPP1;
r) PLAUR and PRELP;
s) PLAUR, IL6, and BMP4;
t) CDH3 and PRELP;
u) IL6 and NRTN;
v) CDH3, IL18 and FGF17;
w) IL6 and NECTIN1;
x) CDH3 and NECTIN1;
y) PRELP and IL6; and
z) CDH3, CCL11 and IL12B.

According to some embodiments, the expression level is selected from protein expression level and mRNA expression level.

According to some embodiments, the expression level is protein expression level and the reagents are antibodies.

According to some embodiments, the expression level is mRNA expression level and the reagents are isolated oligonucleotides, each oligonucleotide specifically hybridizing to a nucleic acid sequence of at least one of the factors.

According to some embodiments, the kit further comprises any one of: (i) a detectable tag or label, (ii) a secondary reagent for detection of the specific reagent, (iii) a solution for rendering a protein susceptible to binding or an mRNA susceptible to hybridization, (iv) a solution for lysing cells, (v) a solution for the purification of proteins or nucleic acids, (vi) any combination thereof.

According to some embodiments, the kit further comprises at least one reagent adapted to specifically determine the expression level of a control.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: An overall workflow of proteomic data collection and analysis. Blood samples were collected at a first time point relative to treatment with immunotherapy (T0) and at a second time point relative to the treatment with immunotherapy (T1) from a cohort of NSCLC patients. The plasma proteome of each sample was profiled using an antibody array. The data were then analyzed in order to discover a proteomic signature that can be used to predict response to treatment. Further analysis of the proteins and the pathways that underlie the resistance to immunotherapy enabled the identification of potential targets for intervention.

FIGS. 2A-2C: Stable proteins discovered using repeated SEMMS with subsets of −75% of the patients. (2A) Of the 300 repeats, 171 converged into models with AUC between 0.5 and 1. Of these, the large majority had AUC between 0.85 and 0.95. (2B) Each of these models included 1 to 4 proteins as predictors. (2C) uPAR (PLAUR) emerged as a highly stable protein, as it appeared in 96% of the models. Other proteins were less stable with IL-6, BMP-4, P-Cadherin, PRELP, Neurturin and XIAP having 12-23% recurrence. Dashed line indicates 10% threshold for protein selection.

FIGS. 3A-3C: Stable proteins discovered using repeated L2N with subsets of −75% of the patients. (3A) Of the 300 repeats, 205 converged into models with AUC between 0.5 and 1. Of these, the large majority had AUC between 0.85 and 0.95. (3B) Each of these models included 2 to 14 proteins as predictors. (3C) IL-6, BMP-4, Neurturin and Cardiotrophin 1 emerged as highly stable proteins as they appeared in >80% of the models. The proteins 1-309, MMP-7, RANK, Arginase 1, Inhibin A, EphB3, FKBP51, DFF45, Nesfatin-1, Cathepsin V, Insulin R, XIAP, Nectin-1, CEACAM-5, Calreticulin-2, R-Spondin 2, Kallikrein 7 had >10% recurrence. Dashed line indicates 10% threshold for protein selection.

FIG. 4: Venn diagram of proteins discovered by SEMMS and L2N when using a threshold of 10% of the models.

FIG. 5: Patient fold-change for the 10 proteins with the highest ROC AUCs. Left dots represent non-responders (NR) and right dots represent responders (R). The lines indicate the median of each group. P-values represent two sample t-test with no correction for multiple comparisons. Zero FC values occur when T0 and T1 measures are below the limit of detection.

FIGS. 6A-6C: Prediction using linear SVM with 4-fold validation with uPAR as a single predictor. (6A) SVM with 4-fold validation yielded an AUC of 0.871. (6B) Waterfall plot displaying the model's predicted response probability for each patient, cutoff is presented at response probability=0.5. Above and below this line the patient is predicted to be a responder or non-responder, respectively. Actual patient responses are indicated by color (light gray=responder, dark gray=non-responder). (6C) At threshold=0.5: accuracy is 0.788, sensitivity is 0.900, specificity is 0.636, PPV is 0.771 and NPV is 0.824. Note: Since threshold=0.5 overlapped with sensitivity=0.9, the confusion diagram represents both conditions. TP—true positive; FN—false negative; TN—true negative; FP—false positive, PPV—positive predictive value, NPV—negative predictive value.

FIGS. 7A-7D: Prediction using linear SVM with 4-fold validation with P-Cadherin as a single predictor. (7A) SVM with 4-fold validation yielded an AUC of 0.738. (7B) Waterfall plot displaying the model's predicted response probability for each patient, cutoff is presented at response probability=0.5. Above and below this line the patient is predicted to be a responder or non-responder, respectively. Actual patient responses are indicated by color (light gray=responder, dark gray=non-responder). (7C) At threshold=0.5: accuracy=0.673, sensitivity=0.767, specificity=0.545, PPV=0.697 and NPV=0.632. (7D) Sensitivity of 0.9 was achieved at threshold=0.408: accuracy=0.654, specificity=0.318, PPV=0.643, NPV=0.700. TP—true positive; FN—false negative; TN—true negative; FP—false positive, PPV—positive predictive value, NPV—negative predictive value.

FIGS. 8A-8D: Prediction using linear SVM with 4-fold validation with uPAR and IL-6 as predictors. (8A) SVM with 4-fold validation yielded an AUC of 0.905. (8B) Waterfall plot display of the model-based prediction of response probability for each patient. Cutoff is presented at response probability=0.5. Above and below this line the patient is predicted to be a responder or non-responder, respectively. Actual patient responses are indicated by color (light gray=responder, dark gray=non-responder). (8C) At threshold=0.5: accuracy=0.808, sensitivity=0.867, specificity=0.727, PPV=0.812 and NPV=0.800. (8D) Sensitivity of 0.9 was achieved at threshold=0.481: accuracy=0.827, specificity=0.727, PPV=0.818, NPV=0.842. TP—true positive; FN—false negative; TN—true negative; FP—false positive, PPV—positive predictive value, NPV—negative predictive value.

FIGS. 9A-9D: Prediction using linear SVM with 4-fold validation with uPAR, IL-6, PRELP, XIAP, and P-Cadherin as predictors. (9A) SVM with 4-fold validation yielded an AUC of 0.952. (9B) Waterfall plot display of the model-based prediction of response probability for each patient. Cutoff is presented at response probability=0.5. Above and below this line the patient is predicted to be a responder or non-responder, respectively. Actual patient responses are indicated by color (light gray=responder, dark gray=non-responder). (9C) At threshold=0.5: accuracy=0.865, sensitivity=0.833, specificity=0.909, PPV=0.926 and NPV=0.800. (9D) Sensitivity of 0.9 was achieved at threshold=0.483: accuracy=0.904, specificity=0.909, PPV=0.931, NPV=0.870. TP—true positive; FN—false negative; TN—true negative; FP—false positive, PPV—positive predictive value, NPV—negative predictive value.

FIG. 10: Principal component analysis of the Cohort B New 1, 2 and Cohort A T0 and T1 data.

FIG. 11: Principal component analysis of the Cohort B New 1, 2 and Cohort A T1/T0 data.

FIG. 12: Measurability of the selected proteins over multiple experimental datasets. Proteins were sorted by percent of measurements in the highest confidence range in the dataset. Measurement quality was divided into four categories: The highest confidence is the concentration range where measurement is linear; measurements below the limit of detection (LOD) are highly inaccurate; measurements near the LOD and above the maximum are non-linear but remain stable. The sum of percent in all four categories is 100% per protein.

FIG. 13: Measurement stability at TO over multiple experimental datasets. Left pane: Cohort B New 1, Middle pane: Cohort B New 2, Right pane: Cohort A. Dots and Xs—measured value per patient (bots—above LOD, Xs—below LOD). Dashed lines: best confidence region. Solid line: limit of detection (LOD). For zero measurements, log is undefined and appears as -inf. The X marks indicate proteins that were excluded from the models due to low measurability and inter-dataset differences.

FIGS. 14A-14D: Validation of linear SVM model trained on Cohort A dataset using P-Cadherin, Nectin-1 as predictors on Cohort B New 1+2 full dataset. (14A) SVM yielded an AUC of 0.795. (14B) Waterfall plot display of the model-based prediction of response probability for each patient. Cutoff is presented at response probability=0.5. Above and below this line the patient is predicted to be a responder or non-responder, respectively. Actual patient responses are indicated by color (light gray=responder, dark gray=non-responder). (14C) At threshold=0.5: accuracy=0.695, sensitivity=0.815, specificity=0.636, PPV=0.524 and NPV=0.875. (14D) Sensitivity of 0.9 was achieved at threshold=0.333: accuracy=0.622, specificity=0.473, PPV=0.490, NPV=0.929. TP—true positive; FN—false negative; TN—true negative; FP—false positive, PPV—positive predictive value, NPV—negative predictive value.

FIGS. 15A-15D: Validation of linear SVM model trained on Cohort A dataset using P-Cadherin, IL-18, and FGF-17 as predictors on New 1+2 full dataset. (15A) SVM yielded an AUC of 0.834. (15B) Waterfall plot display of the model-based prediction of response probability for each patient. Cutoff is presented at response probability=0.5. Above and below this line the patient is predicted to be a responder or non-responder, respectively. Actual patient responses are indicated by color (light gray=responder, dark gray=non-responder). (15C) At threshold=0.5: accuracy=0.756, sensitivity=0.815, specificity=0.727, PPV=0.595 and NPV=0.889. (15D) Sensitivity of 0.9 was achieved at threshold=0.463: accuracy=0.598, specificity=0.436, PPV=0.446, NPV=0.923. TP—true positive; FN—false negative; TN—true negative; FP—false positive, PPV—positive predictive value, NPV—negative predictive value.

FIGS. 16A-16D: Validation of linear SVM model trained on Cohort A dataset using P-Cadherin, PRELP as predictors on New 1+2 full dataset. (16A) SVM yielded an AUC of 0.774. (16B) Waterfall plot display of the model-based prediction of response probability for each patient. Cutoff is presented at response probability=0.5. Above and below this line the patient is predicted to be a responder or non-responder, respectively. Actual patient responses are indicated by color (light gray=responder, dark gray=non-responder). (16C) At threshold=0.5: accuracy=0.634, sensitivity=0.815, specificity=0.545, PPV=0.468 and NPV=0.857. (16D) Sensitivity of 0.9 was achieved at threshold=0.254: accuracy=0.524, specificity=0.327, PPV=0.403, NPV=0.900. TP—true positive; FN—false negative; TN—true negative; FP—false positive, PPV—positive predictive value, NPV—negative predictive value.

FIG. 17: Patient fold-change of the proteins used in the final signatures and that of uPAR in the validation set. Left dots designates non-responders (NR) and right dots designates responders (R). The lines indicate the median of each group. P-values represent two-sample t-test with no correction for multiple comparisons; p-value is not indicated when p>0.1.

FIGS. 18A-18G: Cohort B characteristic. (18A) Overall, 82 NSCLC patients are included in the study, divided into training set (n=33) and validation set (n=49). The response (18B), sex (18C), NSCLC type (18D), α-PD1 type (18E), line of treatment (18F) and age (18G) distributions are indicated.

FIGS. 19A-19B: The performance of the 3-protein signature. (19A) Receiver operating characteristics (ROC) curve indicates a strong predictive power with an area under the curve (AUC) of 0.92 and 0.84 in the training and the validation sets, respectively. The dots indicate the optimal points in the ROC curve when the sensitivity is above 0.9 and the accuracy is at the maximum, respectively. CI, confidence interval (95%). (19B) Sankey plots that show the results of the confusion matrix at threshold=0.5 (maximal accuracy) in each set.

FIGS. 20A-20B: Deep examination of the 3 proteins that comprise the signature. (20A) Dot plots represent the protein expression values in responders and non-responders in each of the signature proteins. Right dots and left dots designate responders and non-responders, respectively. (20B) Kaplan Meier plot of each protein in the signature. Light grey and dark grey designate the overall survival of patients with expression values below or above the median, respectively. R, responders. NR, non-responders.

FIGS. 21A-21B: Selected significantly enriched pathways (FDR p-value <0.05) in non-responders (21A) and responders (21B). The axis represents −log 10 p-values. The different categories are divided into five main groups.

FIG. 22: A multilayer analysis enables the identification of potential targets for intervention. Each column represents a single DEP.

### DETAILED DESCRIPTION OF THE INVENTION

The present invention, in some embodiments, provides methods for determining a therapeutic response to immunotherapy in a subject suffering from cancer. A kit comprising reagents adapted to specifically determine expression levels is also provided.

By a first aspect, there is provided a method for predicting a therapeutic response to immunotherapy in a subject suffering from lung cancer, the method comprising:
a. determining an expression level of at least one factor in a sample obtained from the subject at a first time point relative to the immunotherapy; and
b. determining an expression level of the at least one factor in a sample obtained from the subject at a second time point relative to the immunotherapy;
   wherein a change in expression level of the at least one factor in the subject is indicative of the subject being a non-responder or responder to the immunotherapy, thereby predicting a therapeutic response to immunotherapy in a subject.

By another aspect, there is provided a method for predicting a therapeutic response to immunotherapy in a subject suffering from lung cancer, the method comprising:
a. determining an expression level of at least two factors in a sample obtained from the subject at a first time point relative to the immunotherapy;
b. determining an expression level of the at least two factors in a sample obtained from the subject at a second time point relative to the immunotherapy; and
c. analyzing the determined expression levels with a machine learning classifier, thereby predicting a therapeutic response to immunotherapy in a subject.

By another aspect, there is provided a method for predicting a therapeutic response to immunotherapy in a subject suffering from lung cancer, the method comprising:
a. determining an expression level of at least two factors in a sample obtained from the subject at a first time point relative to the immunotherapy;
b. determining an expression level of the at least two factors in a sample obtained from the subject at a second time point relative to the immunotherapy;
c. calculating a fold-change in expression of the at least two factor from the first time point to the second time point; and
d. analyzing the calculated fold-changes with a machine learning classifier, thereby predicting a therapeutic response to immunotherapy in a subject.

In some embodiments, the immunotherapy is the initiation of the immunotherapy. In some embodiments, the immunotherapy is the start of the immunotherapy. In some embodiments, the immunotherapy is the first dose of the immunotherapy. In some embodiments, the first time point is a time point before the immunotherapy. In some embodiments, the second time point is a time point after the immunotherapy. In some embodiments, determining expression levels at a first time point is before the initiation of the immunotherapy treatment, and determining expression levels at a second time point is after the initiation of the immunotherapy treatment.

In some embodiments, the method is a diagnostic method. In some embodiments, the method is a computer implemented method. In some embodiments, the method is an in vitro method. In some embodiments, the method is an ex vivo method. In some embodiments, the method is for determining response to immunotherapy. In some embodiments, the method is for predicting response to immunotherapy. In some embodiments, predicting is determining. In some embodiments, determining is predicting. In some embodiments, the method is for determining if a subject is a responder to the immunotherapy. In some embodiments, the method is for determining if a subject is a non-responder to the immunotherapy. In some embodiments, the method is for predicting a subject's response to an immunotherapy. In some embodiments, the method is for monitoring response to the immunotherapy. In some embodiments, the method is for determining if the immunotherapy should continue. In some embodiments, the method is for determining if the immunotherapy should end.

Response to immunotherapy may be binary, e.g., positive/negative, and/or expressed in discrete categories, e.g., on a scale of 1-5. In some embodiments, determining response to immunotherapy may be expressed in a binary label, e.g., as ‘yes/no,’ ‘responsive/non-responsive,’ or ‘favorable/non-favorable response.’ In some embodiments, determining response to immunotherapy may be expressed by values indicating a response probability (e.g., at a scale of 1-100%, or 0 to 1). In some embodiments, determining response to immunotherapy may be expressed on a scale and/or be associated with a confidence parameter. Accordingly, in some embodiments, determining response to therapy of the present disclosure may provide for predicting a response rate and/or success rate of a specified treatment in a patient, e.g., the likelihood of a favorable response of a patient to the specified treatment or therapy. For example, in some embodiments, the prediction may be expressed in discrete categories and/or on a scale comprising ‘complete response’, ‘partial response’, ‘stable disease’, ‘progressive disease’, ‘pseudo-progression’, and ‘hyper-progression disease’. In some embodiments, additional and/or other scales and/or thresholds and/or response criteria may be used, e.g., a gradual scale of 1 (non-responsive) to 5 (responsive). In some embodiments, the prediction may indicate the probability of response to immunotherapy. In some embodiments, the prediction may indicate resistance of the patient to immunotherapy.

As used herein, the term “therapy”, “anticancer therapy”, “anti-cancer treatment”, “cancer therapy modality”, “treatment modality”, “cancer treatment”, or “anti-cancer treatment”, as used herein, refer to any method of treatment of cancer in a cancer patient including radiotherapy; chemotherapy; targeted therapy, immunotherapy (immune checkpoint inhibitors, immune checkpoint modulators, adoptive-cell transfer therapy, oncolytic viruses therapy, treatment vaccines, immune system modulators and monoclonal antibodies), hormonal therapy, anti-angiogenic therapy and photodynamic therapy; thermotherapy and surgery or a combination thereof.

In some embodiments, the immunotherapy is a single immunotherapy. In some embodiments, the immunotherapy is a combination of more than one type of immunotherapy. In some embodiments, the immunotherapy is a plurality of immunotherapies. In some embodiments, the immunotherapy is in combination with other therapies. In some embodiments, the other therapy is another anticancer treatment. Examples of other anticancer treatments include but are not limited to chemotherapy, radiation, surgery, and targeted therapy. Any other anticancer treatment may be combined. In some embodiments, the immunotherapy is in combination with chemotherapy. In some embodiments, the immunotherapy is in combination with targeted therapy. In some embodiments, the immunotherapy is combined with more than one type of an additional immunotherapy. In some embodiments, the immunotherapy is immune checkpoint blockade. In some embodiments, the immunotherapy is immune checkpoint protein inhibition. In some embodiments, the immunotherapy is immune checkpoint modulation. In some embodiments, immune checkpoint blockade and/or immune checkpoint inhibition and/or immune checkpoint modulation comprises administering to the subject an immune checkpoint inhibitor (ICI).

As used herein, the term “an immune checkpoint inhibitor (ICI)” refers to a single ICI, a combination of more than one type of ICI and a combination of an ICI with another cancer therapy. The ICI may be a monoclonal antibody, a humanized antibody, a fully human antibody, a bispecific antibody, a fusion protein, or a combination thereof. Any known method or compound used for ICI may be used as part of a method of the invention.

In some embodiments, the immune checkpoint protein is selected from PD-1 (Programmed Death-1) PD-L1, PD-L2; CTLA-4 (Cytotoxic T-Lymphocyte-Associated protein 4); A2AR (Adenosine A2A receptor), also known as ADORA2A; BT-H3, also called CD276; BT-H4, also called VTCN1; BT-H5; BTLA (B and T Lymphocyte Attenuator), also called CD272; IDO (Indoleamine 2,3-dioxygenase); KIR (Killer-cell Immunoglobulin-like Receptor); LAG-3 (Lymphocyte Activation Gene-3); TDO (Tryptophan 2,3-dioxygenase); TIM-3 (T-cell Immunoglobulin domain and Mucin domain 3); NOX2 (nicotinamide adenine dinucleotide phosphate NADPH oxidase isoform 2); SIGLEC7 (Sialic acid-binding immunoglobulin-type lectin 7), also called CD328; SIGLEC9 (Sialic acid-binding immunoglobulin-type lectin 9), also called CD329, OX40, TIGIT and VISTA (V-domain Ig suppressor of T cell activation). In some embodiments, the immune checkpoint protein is selected from PD-1, PD-L1 and PD-L2. In some embodiments, the immune checkpoint protein is selected from PD-1 and PD-L1. In some embodiments, the immune checkpoint protein is PD-1. In some embodiments, immune checkpoint blockade comprises an anti-PD-1/PD-L1/PD-L2 immunotherapy. In some embodiments, immune checkpoint blockade comprises an anti-PD-1 immunotherapy. In some embodiments, the immunotherapy is anti-PD-L1 therapy. In some embodiments, the immunotherapy is anti-CTLA-4 therapy. In some embodiments, the immunotherapy is anti-PD-1 and anti-CTLA-4 therapy. In some embodiments, the immunotherapy is anti-PD-L1 and anti-CTLA-4 therapy. In some embodiments, the immunotherapy is selected from anti-PD-1/PD-L1 therapy, anti-CTLA-4 therapy, and both.

In some embodiments, immune checkpoint blockade comprises an anti-PD-1 and/or anti-PD-L1 immunotherapy. In some embodiments, immune checkpoint blockade comprises an anti-PD-1 and/or anti-CTLA-4 immunotherapy. In some embodiments, the immunotherapy is a blocking antibody. In some embodiments, the immunotherapy is administration of a blocking antibody to the subject.

In some embodiments, the ICI is a monoclonal antibody (mAb) against PD-1 or PD-L 1. In some embodiments, the ICI is a mAb that neutralizes/blocks the PD-1 pathway. In some embodiments, the ICI is a mAb against PD-1. In some embodiments, the anti-PD-1 mAb is Pembrolizumab (Keytruda; formerly called lambrolizumab). In some embodiments, the anti-PD-1 mAb is Nivolumab (Opdivo). In some embodiments, the anti-PD-1 mAb is Pidilizumab (CT0011). In some embodiments, the anti-PD-1 mAb is any one of REGN2810, AMP-224, MEDI0680, or PDR001. In some embodiments, the ICI is a mAb against PD-L1. In some embodiments, the anti-PD-L1 mAb is selected from Atezolizumab (Tecentriq), Avelumab (Bavencio), and Durvalumab (Imfinzi). In some embodiments, the ICI is a mAb against CTLA-4. In some embodiments, the anti-CTLA-4 mAb is ipilimumab.

In some embodiments, the immunotherapy is administered in combination with one or more conventional cancer therapy including chemotherapy, targeted cancer therapy, steroids and radiotherapy. Combinations of ICI and radiation therapy have been studied in multiple clinical trials. It will be understood by a skilled artisan that the predictive proteins disclosed herein are predictive in immunotherapy as a monotherapy, as well as part of a combination therapy.

In some embodiments, the method further comprises administering the immunotherapy to the subject. In some embodiments, the method further comprises administering the immunotherapy to the subject after the first determining. In some embodiments, the method further comprises administering the immunotherapy to the subject before the second determining. In some embodiments, the method further comprises administering the immunotherapy to the subject after the second determining. In some embodiments, the first determining is immediately before the immunotherapy, or before administration of the immunotherapy. In some embodiments, the first determining is at least 1 hour, 2 hours, 3 hours, 6 hours, 8 hours, 12 hours, 1 day, 2 days, 3 days, 5 days, 1 week, 2 weeks, 3 weeks, 4 weeks or 1 month before the immunotherapy or before administration of the immunotherapy. Each possibility represents a separate embodiment of the invention. In some embodiments, the first determining is at most 1 day, 2 days, 3 days, 5 days, 1 week, 2 weeks, 3 weeks, 4 weeks or 1 month before the immunotherapy or before administration of the immunotherapy. Each possibility represents a separate embodiment of the invention. In some embodiments, the first determining is at most 1 week before the immunotherapy or before administration of the immunotherapy. In some embodiments, the first determining is at most 2 weeks before the immunotherapy or before administration of the immunotherapy. In some embodiments, the first determining is at most 3 weeks before the immunotherapy or before administration of the immunotherapy. In some embodiments, the first determining is at most 1 month before the immunotherapy or before administration of the immunotherapy. In some embodiments, the second determining is at a time after initiation of the immunotherapy, or after administration of the immunotherapy, sufficient for altered expression of the at least one factor. In some embodiments, the time sufficient is at least 1 day, 2 days, 3 days, 5 days, 1 week, 2 weeks, 3 weeks, 4 weeks, 1 month, 2 months, 3 months, 4 months, 5 months, 6 months or a year. Each possibility represents a separate embodiment of the invention. In some embodiments, the second determining is at most 1 week, 2 weeks, 3 weeks, 4 weeks, 1 month, 2 months, 3 months, 4 months, 5 months, 6 months or a year after initiation of the immunotherapy, or after administration of the immunotherapy. Each possibility represents a separate embodiment of the invention. In some embodiments, the first determining is before the initiation of immunotherapy. In some embodiments, the second determining is after a single administration of the immunotherapy. In some embodiments, the second determining is at a time after initiation of the immunotherapy and more than 2 administrations of immunotherapy were applied. In some embodiments, the second determining is at a time after initiation of the immunotherapy after a single treatment with immunotherapy. In some embodiments, the first determining is before the first treatment with immunotherapy, and the second determining is after the first treatment with immunotherapy.

In some embodiments, the subject is a mammal. In some embodiments, the subject is a human. In some embodiments, the subject suffers from lung cancer. In some embodiment, the subject is diagnosed with lung cancer. In some embodiments, the lung cancer is a cancer responsive to the immunotherapy. In some embodiments, the lung cancer is a cancer non-responsive to the immunotherapy. In some embodiments, the lung cancer is a PD-L1 positive cancer. In some embodiments, the lung cancer is a PD-L1 negative cancer. In some embodiments, the lung cancer is non-small cell lung cancer (NSCLC). In some embodiments, the lung cancer is small cell lung cancer (SCLC). In some embodiments, the subject is naïve to therapy before the first determining. In some embodiments, the subject has previously been treated by an anti-cancer therapy other than the immunotherapy. In some embodiments, the subject is naïve to any immunotherapy. In some embodiments, the subject has previously been treated by an immunotherapy other than the immunotherapy.

In some embodiments, the expression is protein expression. In some embodiments, a factor is a protein. In some embodiments, a factor is a gene. In some embodiments, protein expression is soluble protein expression. In some embodiments, the protein expression is metabolic protein expression. In some embodiments, the protein expression is membranal protein expression. In some embodiments, the protein expression is secreted protein expression. In some embodiments, the protein expression is cellular protein expression. In some embodiments, the expression is mRNA expression. In some embodiments, the expression is protein expression or mRNA expression. The terms “expression” and “expression levels” are used herein interchangeably and refer to the amount of a gene product (e.g., mRNA and/or protein) present in the sample. In some embodiments, determining comprises quantification of expression levels. Determining of the expression level of the factor can be performed by any method known in the art. Methods of determining protein expression include, for example, antibody arrays, immunoblotting, immunohistochemistry, flow cytometry (FACS), enzyme-linked immunosorbent assay (ELISA), Western blotting, proteomics arrays, proximity extension assay (PEA) proteomics arrays, proteome sequencing, flow cytometry (CyTOF), aptamer-based assays, multiplex assays, mass spectrometry and chromatography. In some embodiments, determining protein expression levels comprises ELISA. In some embodiments, determining protein expression levels comprises protein array hybridization. In some embodiments, determining protein expression levels comprises mass-spectrometry quantification. In some embodiments, determining protein expression levels comprises targeted mass spectrometry. In some embodiments, determining protein expression levels comprises untargeted mass spectrometry. In some embodiments, determining protein expression levels comprises shotgun proteomics using mass spectrometry. In some embodiments, determining protein expression levels comprises top-down mass spectrometry. In some embodiments, determining protein expression levels comprises bottom-up mass spectrometry. In some embodiments, determining protein expression levels comprises data-independent acquisition (DIA) mass spectrometry. In some embodiments, determining protein expression levels comprises data-dependent acquisition (DDA) mass spectrometry. In some embodiments, determining protein expression levels comprises PEA. In some embodiments, determining protein expression levels comprises aptamer-based assays. Methods of determining mRNA expression include, for example polymerase chain reaction (PCR), reverse transcriptase PCR (RT-PCR), quantitative PCR, real-time PCR, digital PCR, microarrays, RNA sequencing, single-cell RNA sequencing, northern blotting, in situ hybridization, next generation sequencing, deep sequencing and massively parallel sequencing. In some embodiments, determining expression levels is by a combination of any of the methods for determining protein and RNA.

In some embodiments, the determining is directly in the sample. In some embodiments, the determining is in the unprocessed sample. In some embodiments, the determining is in a processed sample. In some embodiments, the method further comprises processing the sample. In some embodiments, processing comprises isolating protein from the sample. In some embodiments, processing comprises isolating nucleic acids from the sample. In some embodiments, the nucleic acid is RNA. In some embodiments, the RNA is mRNA. In some embodiments, the processing comprises lysing cells in the sample.

In some embodiments, the sample is a biological sample. Biological samples may include any type of biological sample obtained from an individual, including body tissues, body fluids, body excretions, exhaled breath, or other sources. In some embodiments, the biological fluid is selected from, blood, plasma, lymph, cerebral spinal fluid, urine, feces, semen, tumor fluid and gastric fluid. In some embodiments, the biological sample is a tumor. In some embodiments, the sample is not a tumor sample. In some embodiments, the sample is a fluid. In some embodiments, the fluid is a biological fluid. In some embodiments, a biological fluid is selected from whole blood, blood plasma, blood serum, peripheral blood mononuclear cells (PBMCs), lymph, urine, saliva, semen, synovial fluid and spinal fluid. In some embodiments, the biological fluid may be fresh or frozen. In some embodiments, the biological sample is selected from blood plasma, whole blood, blood serum, cerebrospinal fluid (CSF), and PBMCs. In some embodiments, the sample is from the subject. In some embodiments, the sample is not a tumor sample. In some embodiments, the sample is not a hematopoietic cancer and the sample is a blood sample. In some embodiments, the sample is a sample that does not comprise cancer cells. In some embodiments, the biological sample is circulating tumor cells. In some embodiments, the sample comprises circulating tumor cells. In some embodiments, a blood sample comprises a peripheral blood sample and a plasma sample. In some embodiments, the sample is a plasma sample. In some embodiments, processing comprises isolating plasma. In some embodiments, the sample obtained at the first time point, and the sample obtained at the second time point are the same type of sample. In some embodiments, the sample obtained at the first time point, and the sample obtained at the second time point are different types of samples. In some embodiments, the biological sample is blood plasma. In some embodiments, the biological sample is CSF. In some embodiments, the biological sample is PBMCs. In some embodiments, the biological sample is a blood sample. In some embodiments, blood is peripheral blood.

In some embodiments, expression of at least one factor is determined. In some embodiments, expression of a plurality of factors is determined. In some embodiments, expression of at least 1, 2, 3, 4, 5, 6, 7, 8, 9, 10, 11, 12, 13, 14, 15, 16, 17, 18, 19, 20, 21, 22, 23, 24 or 25 factors is determined. Each possibility represents a separate embodiment of the invention. In some embodiments, the factor determined at the first time point is the same factor as is determined at the second time point. In some embodiments, the expression level is a sum of expression levels of the measured factors. In some embodiments, the expression level is a multiplicity of expression levels of the measured factors. In some embodiments, the expression level is a ratio of expression levels of the measured factors. In some embodiments, the expression level is the expression level of each factor determined. In some embodiments, the expression levels of a factor are weighted. In some embodiments, a factor in a signature is given increased or decreased weight.

In some embodiments, the analyzing comprises calculating the change in expression. In some embodiments, the analyzing comprises calculating the change in expression from the determining at a first time point and the determining at the second time point. In some embodiments, the analyzing comprises calculating the change in expression from the determining before initiation and the determining after initiation. In some embodiments, the change in expression is the change from the first time point to the second time point. In some embodiments, the change in expression is a fold-change. In some embodiments, the change in expression is the log of the fold-change. In some embodiments, the change in expression is the log of expression at the second time point divided by the expression at the first time point. In some embodiments, the change in expression is the log of expression after initiation divided by the expression before initiation. In some embodiments, the change in expression is indicative of a responder or a non-responder. In some embodiments, the change in expression is analyzed with the machine learning classifier. In some embodiments, the classifier classifies changes in expression of a plurality of factors. In some embodiments, the classifier classifies changes in expression of a signature.

In some embodiments, expression of a control factor is determined. In some embodiments, the method further comprises determining expression levels of a control in the sample obtained at the first time point. In some embodiments, the method further comprises determining expression levels of a control in the sample obtained at the second time point. In some embodiments, the control factor at the first time point is the same control factor as at the second time point. In some embodiments, the control factor at the first and second time points are different. In some embodiments, the method further comprises determining expression levels of a control in the sample obtained before immunotherapy. In some embodiments, the method further comprises determining expression levels of a control in the sample obtained after immunotherapy. In some embodiments, the expression level of the control is measured in both samples. In some embodiments, the expression of the at least one factor is normalized to expression of the control. In some embodiments, the control is used to confirm the quality of the sample or of the data produced from the sample. In some embodiments, the control is a housekeeping gene/protein. Housekeeping genes/proteins are well known in the art and any such gene/protein may be used as a control. Generally, housekeeping genes/proteins are constitutively expressed, easily measured and play a role in an essential cellular function. In some embodiments, the control is a protein other than the at least one factor. In some embodiments, expression of the control is the same in responders and non-responders. In some embodiments, expression of the control does not change from the before to after immunotherapy. In some embodiments, the same is substantially the same. In some embodiments, does not change is does not substantially change. In some embodiments, substantially is not more than a 10% difference or change. In some embodiments, substantially is not more than a 5% difference or change. In some embodiments, the control is determined in a sample other than the sample used to determine the expression level of the factor. In some embodiments, the control is a clinical, demographic, and/or physical information.

In some embodiments, the expression is normalized. In some embodiments, the change in expression is normalized. In some embodiments, the normalization is to a median expression value. In some embodiments, the fold-change is normalized. In some embodiments, the normalization is to a median fold-change. In some embodiments, the normalization is to a median log fold-change. In some embodiments, all expressions are normalized. In some embodiments, all fold-changes are normalized. In some embodiments, all log fold-changes are normalized.

In some embodiments, the factor is selected from those provided in Table 2. In some embodiments, the factor is selected from those provided in Table 3. In some embodiments, the factor is selected from those provided in Table 4. In some embodiments, the factor is selected from those provided in Table 5. In some embodiments, the factor is selected from those provided in Table 7. In some embodiments, the factor is selected from those provided in Table 10. In some embodiments, the factor is selected from the group consisting of uPAR, P-Cadherin, IL-6, Nectin-1, VEGF-D, BMP-4, XIAP, IL-2, PRELP, FGF-17, CA125, Periostin, MSP, CK18, Ephrin-A4, IL-4 Ra, Granulysin, IL-18, B4GalT1, BMP-9, Semaphorin 4D, Epo R, EphB3, HGF, IFNab R2, OPN, FLRT1, B7-H2, Notch-3, Neurturin, CHST2, 1-309, CD97, NTAL, Draxin, IGFBP-4, TAFA5, IGFBP-5, RCOR1, CCL11, and IL12B, Cystatin B, Nesfatin, PP and DFF45. In some embodiments, the factor is selected from the group consisting of uPAR, P-Cadherin, IL-6, Nectin-1, VEGF-D, BMP-4, XIAP, IL-2, PRELP, FGF-17, CA125, Periostin, MSP, CK18, Ephrin-A4, IL-4 Ra, Granulysin, IL-18, B4GalT1, BMP-9, Semaphorin 4D, Epo R, EphB3, HGF, IFNab R2, OPN, FLRT1, B7-H2, Notch-3, Neurturin, CHST2, 1-309, CD97, NTAL, Draxin, IGFBP-4, TAFA5, IGFBP-5, RCOR1, CCL11, and IL12B, Cystatin B, and Nesfatin. In some embodiments, the factor is selected from the group consisting of uPAR, P-Cadherin, IL-6, Nectin-1, VEGF-D, BMP-4, XIAP, IL-2, PRELP, FGF-17, CA125, Periostin, MSP, CK18, Ephrin-A4, IL-4 Ra, Granulysin, IL-18, B4GalT1, BMP-9, Semaphorin 4D, Epo R, EphB3, HGF, IFNab R2, OPN, FLRT1, B7-H2, Notch-3, CHST2, 1-309, CD97, NTAL, Draxin, IGFBP-4, TAFA5, IGFBP-5, RCOR1, CCL11, and IL12B. In some embodiments, the factor is selected from the group consisting of uPAR, P-Cadherin, IL-6, Nectin-1, VEGF-D, BMP-4, XIAP, IL-2, PRELP, FGF-17, CA125, Periostin, MSP, CK18, Ephrin-A4, IL-4 Ra, Granulysin, IL-18, B4GalT1, BMP-9, Semaphorin 4D, Epo R, EphB3, HGF, IFNab R2, OPN, FLRT1, B7-H2, Notch-3, CHST2, 1-309, CD97, NTAL, Draxin, IGFBP-4, TAFA5, IGFBP-5, RCOR1, Neurturin, CCL11, and IL12B. In some embodiments, the factor is selected from the group consisting of CA125, IL-6, B7-H2, B4GalT1, Cystatin B, OPN, P-Cadherin, Nesfatin-1, PP, uPAR, and DFF45. The factors described herein are well known and their nucleic acid sequence and amino acid sequence are well known and can be accessed from numerous databases, including, but not limited to Uniprot, NCBI, and UCSC Genome Browser. As numerous genes/proteins have multiple names, Table 1 provides the names of the proteins referred to herein, as well as their official name, the Uniprot accession number for the human protein, and the gene name. It will be understood that these various names are interchangeable and a reference to one name of a protein is the same as using any other known name of the protein. For example, a reference to uPAR and a reference to PLAUR are both referring to the same protein and there should be no distinction applied to the use of one name or the other. Table 1 can be used to determine if two different names indeed refer to the same protein.

TABLE 1
Factors recited herein.

| Target name | Protein name | Uniprot ID | Gene name |
|---|---|---|---|
| uPAR | PLAUR | Q03405 | PLAUR |
| IL-6 | IL6 | P05231 | IL6, IFNB2 |
| P-Cadherin | CDH3 | P22223 | CDH3, CDHP |
| PRELP | PRELP | P51888 | PRELP, SLRR2A |
| Neurturin | NRTN | Q99748 | NRTN |
| XIAP | XIAP | P98170 | XIAP, API3, BIRC4, |
| | | | IAP3 |
| Cardiotrophin-1 | CTF1 | Q16619 | CTF1 |
| I-309 | CCL1 | P22362 | CCL1, SCYA1 |
| MMP-7 | MMP7 | P09237 | MMP7, MPSL1, PUMP1 |
| RANK | TNFRSF11A | Q9Y6Q6 | TNFRSF11A, RANK |
| Arginase 1 | ARG1 | P05089 | ARG1 |
| Calreticulin-2 | CALR3 | Q96L12 | CALR3, CRT2 |
| Inhibin A | INHA/ | P05111/ | INHA/INHBA |
| | INHBA | P08476 | |
| EphB3 | EphB3 | P54753 | EPHB3, ETK2, HEK2, |
| | | | TYRO6 |
| Nesfatin-1 | NUCB2 | P80303 | NUCB2, NEFA |
| R-Spondin 2 | RSPO2 | Q6UXX9 | RSPO2, |
| | | | UNQ9384/PRO34209 |
| Cathepsin V | CTSV | O60911 | CTSV, CATL2, CTSL2, |
| | | | CTSU, |
| | | | UNQ268/PRO305 |
| Insulin R | INSR | P06213 | INSR |
| Kallikrein 7 | KLK7 | P49862 | KLK7, PRSS6, SCCE |
| XIAP | XIAP | P98170 | XIAP |
| CEACAM-5 | CEACAM5 | P06731 | CEACAM5 |
| BMP-4 | BMP4 | P12644 | BMP4, BMP2B, DVR4 |
| BMP-9 | GDF2 | Q9UK05 | GDF2, BMP9 |
| OPN | SPP1 | P10451 | SPP1, BNSP, OPN, |
| | | | PSEC0156 |
| MSP | MST1 | P26927 | MST1, D3F15S2, |
| | | | DNF15S2, HGFL |
| VEGF-D | FIGF | O43915 | FIGF, VEGFD |
| CK18 | KRT18 | P05783 | KRT18, CYK18, PIG46 |
| EphB3 | EphB3 | P54753 | EPHB3, ETK2, HEK2, |
| | | | TYRO6 |
| Nectin-1 | NECTIN1 | Q15223 | NECTIN1, HVEC, |
| | | | PRR1, PVRL1 |
| CA9 | CA9 | Q16790 | CA9, G250, MN |
| CA19-9 | — | — | |
| Semaphorin 4D | SEMA4D | Q92854 | SEMA4D, C9orf164, |
| | | | CD100, SEMAJ |
| B7-H2 | ICOSLG | O75144 | ICOSLG, B7H2, B7RP1, |
| | | | ICOSL, KIAA0653 |
| Cystatin E M | CST6 | Q15828 | CST6 |
| CD97 | CD97 | P48960 | CD97 |
| CA125 | MUC16 | Q8WXI7 | MUC16, CA125 |
| Draxin | DRAXIN | Q8NBI3 | DRAXIN, C1orf187, |
| | | | PSEC0258, |
| | | | UNQ3119/PRO10268 |
| B4GalT1 | B4GALT1 | P15291 | B4GALT1 GGTB2 |
| FGF-17 | FGF17 | O60258 | FGF17, |
| | | | UNQ161/PRO187 |
| Cystatin B | CSTB | P04080 | CSTB, CST6, STFB |
| IL-18 | IL18 | Q14116 | IL18, IGIF, IL1F4 |
| FKBP51 | FKBP5 | Q13451 | FKBP5, AIG6, FKBP51 |
| PRX2 | PRDX2 | P32119 | PRDX2, NKEFB, |
| | | | TDPX1 |
| Serpin B8 | Serpin B8 | P50452 | SERPINB8, PI8 |
| NPDC-1 | NPDC1 | Q9NQX5 | NPDC1 |
| OSCAR | OSCAR | Q8IYS5 | OSCAR |
| PILR-alpha | PILRA | Q9UKJ1 | PILRA |
| Ephrin-A4 | EFNA4 | P52798 | EFNA4, EPLG4, LERK4 |
| HAI-2 | SPINT2 | O43291 | SPINT2, HAI2, KOP |
| PP | PPY | P01298 | PPY, PNP |
| Cortactin | CTTN | Q14247 | CTTN, EMS1 |
| TACI | TNFRSF13B | O14836 | TNFRSF13B, TACI |
| Semaphorin 7A | SEMA7A | O75326 | SEMA7A, CD108, |
| | | | SEMAL |
| TRANCE | TNFSF11 | O14788 | TNFSF11, OPGL, |
| | | | RANKL, TRANCE |
| Cadherin-13 | CDH13 | P55290 | CDH13, CDHH |
| Cerberus 1 | CER1 | O95813 | CER1, DAND4 |
| DFF45 | DFFA | O00273 | DFFA, DFF1, DFF45, |
| | | | H13 |
| FKBP51 | FKBP5 | Q13451 | FKBP5, AIG6, FKBP51 |
| IL-2 | IL2 | P60568 | IL2 |
| Epo R | EPOR | P19235 | EPOR |
| TAFA5 | FAM19A5 | Q7Z5A7 | FAM19A5, TAFA5, |
| | | | UNQ5208/PRO34524 |
| KIR2DL3 | KIR2DL3 | P43628 | KIR2DL3, CD158B2, |
| | | | KIRCL23, NKAT2 |
| BMPR-IB | BMPR1B | O00238 | BMPRIB |
| Semaphorin 6C | SEMA6C | Q9H3T2 | SEMA6C, KIAA1869, |
| | | | SEMAY |
| LEDGF | PSIP1 | O75475 | PSIP1, DFS70, LEDGF, |
| | | | PSIP2 |
| IL-5 | IL5 | P05113 | IL5 |
| Testican 2 | SPOCK2 | Q92563 | SPOCK2, KIAA0275, |
| | | | TICN2, |
| | | | UNQ269/PRO306 |
| IL-5 Ra | IL5RA | Q01344 | IL5RA, IL5R |
| TAFA1 | FAM19A1 | Q7Z5A9 | FAM19A1, TAFA1 |
| Periostin | POSTN | Q15063 | POSTN, OSF2 |
| IL-4 Ra | IL4R | P24394 | IL4R, IL4RA, 582J2.1 |
| Granulysin | GNLY | P22749 | GNLY, LAG2, NKG5, |
| | | | TLA519 |
| HGF | HGF | P14210 | HGF, HPTA |
| IFNab R2 | IFNAR2 | P48551 | IFNAR2, IFNABR, |
| | | | IFNARB |
| FLRT1 | FLRT1 | Q9NZU1 | FLRT1, SPG68 |
| Notch-3 | NOTCH3 | Q9UM47 | NOTCH3 |
| CHST2 | CHST2 | Q9Y4C5 | CHST2, GN6ST |
| NTAL | LAT2 | Q9GZY6 | LAT2, LAB, NTAL |
| | | | WBS15, WBSCR15, |
| | | | WBSCR5, HSPC046 |
| IGFBP-4 | IGFBP4 | P22692 | IGFBP4, IBP4 |
| IGFBP-5 | IGFBP5 | P24593 | IGFBP5, IBP5 |
| RCOR1 | RCOR1 | Q9UKL0 | RCOR1, KIAA0071, |
| | | | RCOR |
| Eotaxin | CCL11 | P51671 | CCL11, SCYA11 |
| IL12p40 | IL12B | P29460 | IL12B |

In some embodiments, a plurality of factors is a signature. In some embodiments, at least two factors are a signature. In some embodiments, determining an expression level comprises determining expression level of a signature. In some embodiments, the method comprises determining the expression level of at least two factors. In some embodiments, the method comprises determining the expression level of a plurality of factors. In some embodiments, the method comprises determining the expression levels of a signature. In some embodiments, the signature is a response signature. In some embodiments, the signature comprises at least a first factor and a second factor. In some embodiments, the signature comprises at least two factors. In some embodiments, the method comprises determining expression level of a signature in the sample obtained at a first time point and the sample obtained at a second time point. In some embodiments, the method comprises determining expression level of a signature in the sample obtained before and the sample obtained after immunotherapy.

In some embodiments, determining the response to therapy in the subject uses additional information such as clinical, demographic, and/or physical information. For example, in some embodiments, such data may include characteristics obtained from the diseased tissue itself (e.g., from a tumor of a cancer patient). In some embodiments, such data may include, but is not limited to: demographic information (sex, age, ethnicity); performance status; hematological and chemistry measurements; cancer disease history, e.g., date of cancer diagnosis, primary cancer type and stage, disease biomarkers (e.g. PD-L1), disease treatment history, histology, TNM stage, assessment of measurable lesions, time of tumor progression, site of recurrence, proposed treatment; general medical history, including smoking history and drinking habits, background diseases including hypertension, diabetes, ischemic heart disease, renal insufficiency, chronic obstructive pulmonary disease, asthma, liver insufficiency, Inflammatory Bowel Disease, autoimmune diseases, endocrine diseases, and others; family medical history; genetic information, e.g. mutations, gene amplifications, and others (e.g. EGFR, BRAF, HER2, KRAS, MAP2K1, MET, NRAS, NTRK1, PIK3CA, RET, ROS1, TP53, ALK, MYC, NOTCH, PTEN, RB1, CDKN2A, KIT, NF1); physical parameters, e.g., temperature, pulse, height, weight, BMI, blood pressure, complete blood count including all examined parameters, liver function, renal function, electrolytes; medication (prescribed and non-prescribed); relative lymphocyte count; neutrophil to lymphocyte ratio; baseline protein levels in the plasma (e.g. LDH); and/or marker staining (e.g. PD-L1 in the tumor or in circulating tumor cells), side effects, adverse events, and time of death.

In some embodiments, the signature is a signature provided in Table 6. In some embodiments, the signature is a signature provided in Table 8. In some embodiments, the signature is a signature provide in Table 9. In some embodiments, the first factor is selected from uPAR, P-Cadherin, IL-6, BMP-4, PRELP and OPN. In some embodiments, the first factor is selected from uPAR, P-Cadherin and IL-6. In some embodiments, the second factor is selected from uPAR, P-Cadherin, IL-6, BMP-4, PRELP, OPN, Neurturin, Nectin-1, IL-18, CCL11, IL12B, and FGF-17. In some embodiments, the second factor is selected from uPAR, P-Cadherin, IL-6, BMP-4, PRELP, XIAP, OPN, Neurturin, Nectin-1, IL-18, CCL11, IL12B, and FGF-17. In some embodiments, the second factor is selected from uPAR, P-Cadherin, IL-6, BMP-4, PRELP, OPN, Neurturin, Nectin-1, CCL11+IL12B and IL-18+FGF-17. In some embodiments, the first factor is uPAR and the second factor is selected from IL-6, PRELP, P-Cadherin, BMP-4 and OPN. In some embodiments, the first factor is uPAR and the second factor is selected from IL-6, PRELP, P-Cadherin, XIAP, BMP-4 and OPN. In some embodiments, the first factor is P-Cadherin and the second factor is selected from IL-6, BMP-4, PRELP, OPN, IL-18, Nectin-1, CCL11, IL12B and FGF-17. In some embodiments, the first factor is P-Cadherin and the second factor is selected from IL-6, BMP-4, PRELP, OPN, IL-18, XIAP, Nectin-1, CCL11, IL12B and FGF-17. In some embodiments, the first factor is P-Cadherin and the second factor is selected from uPAR, IL-6, BMP-4, PRELP, OPN, Nectin-1, CCL11+IL12B and IL-18+FGF-17. In some embodiments, the first factor is IL-6 and the second factor is selected from Nectin-1, PRELP and Neurturin. In some embodiments, the first factor is IL-6 and the second factor is selected from Nectin-1, uPAR, PRELP and Neurturin. In some embodiments, the signature comprises or consists of uPAR, P-Cadherin, IL-6, BMP-4, PRELP and OPN. In some embodiments, the signature comprises or consists of uPAR, PRELP and IL-6. In some embodiments, the signature comprises or consists of uPAR and IL-6. In some embodiments, the signature comprises or consists of P-cadherin, IL-6, BMP-4, PRELP and OPN. In some embodiments, the signature comprises or consists of uPAR and PRELP In some embodiments, the signature comprises or consists of uPAR, IL-6, BMP-4. In some embodiments, the signature comprises or consists of P-cadherin and PRELP. In some embodiments, the signature comprises or consists of IL-6 and Neurturin. In some embodiments, the signature comprises or consists of P-cadherin, IL-18 and FGF-17. In some embodiments, the signature comprises or consists of P-cadherin, Nectin-1, IL-18 and FGF-17. In some embodiments, the signature comprises or consists of IL-6 and Nectin-1. In some embodiments, the signature comprises or consists of P-Cadherin and Nectin-1. In some embodiments, the signature comprises or consists of PRELP and IL-6. In some embodiments, the signature comprises or consists of P-cadherin, CCL11 and IL12B. In some embodiments, the signature comprises or consists of uPAR, IL-6, BMP-4, P-cadherin, PRELP, Neurturin and XIAP. In some embodiments, the signature comprises or consists of IL-6, BMP-4, Neurturin and cardiotrophin-1.

In some embodiments, BMP-4, CA125, CK18, Draxin, EphB3, Ephrin-A4, Granulysin, HGF, 1-309, IGFBP-4, IL-12p40, IL-12B, IL-18, IL-2, IL-4Ra, IL-6, OPN, P-Cadherin, Periostin, Semaphorin 4D, TAFA5, DFF45 and uPAR are increased in non-responders at the second time point. In some embodiments, an increase in expression of any one of BMP-4, CA125, CK18, Draxin, EphB3, Ephrin-A4, Granulysin, HGF, 1-309, IGFBP-4, IL-12p40, IL-12B, IL-18, IL-2, IL-4Ra, IL-6, OPN, P-Cadherin, Periostin, Semaphorin 4D, TAFA5, DFF45 and uPAR is indicative of a subject being a non-responder. In some embodiments, an increase in expression of any one of BMP-4, CA125, CK18, Draxin, EphB3, Ephrin-A4, Granulysin, HGF, 1-309, IGFBP-4, IL-12p40, IL-12B, IL-18, IL-2, IL-4Ra, IL-6, OPN, P-Cadherin, Periostin, Semaphorin 4D, TAFA5 and uPAR is indicative of a subject being a non-responder. In some embodiments, the factor is selected from BMP-4, CA125, CK18, Draxin, EphB3, Ephrin-A4, Granulysin, HGF, 1-309, IGFBP-4, IL-12p40, IL-12B, IL-18, IL-2, IL-4Ra, IL-6, OPN, P-Cadherin, Periostin, Semaphorin 4D, TAFA5, DFF45 and uPAR and an increased expression level of the factor is indicative of the subject being a non-responder to the immunotherapy.

In some embodiments, B4GalT1, B7-H2, BMP-9, CCL11, CD97, CHST2, Epo R, FGF-17, FLRT1, IFNab R2, IGFBP-5, MSP, Nectin-1, Notch-3, NTAL, Neurturin, PRELP, RCOR1, VEGF-D, Cystatin B, Nesfatin, PP and XIAP are increased in responders at the second time point. In some embodiments, B4GalT1, B7-H2, BMP-9, CCL11, CD97, CHST2, Epo R, FGF-17, FLRT1, IFNab R2, IGFBP-5, MSP, Nectin-1, Notch-3, NTAL, Neurturin, PRELP, RCOR1, VEGF-D, Cystatin B, Nesfatin, PP and XIAP are not increased in non-responders at the second time point. In some embodiments, an increase in expression of any one of B4GalT1, B7-H2, BMP-9, CCL11, CD97, CHST2, Epo R, FGF-17, FLRT1, IFNab R2, IGFBP-5, MSP, Nectin-1, Notch-3, NTAL, Neurturin, PRELP, RCOR1, VEGF-D, Cystatin B, Nesfatin, PP and XIAP is indicative of a subject being a responder. In some embodiments, a lack of increase in expression of any one of B4GalT1, B7-H2, BMP-9, CCL11, CD97, CHST2, Epo R, FGF-17, FLRT1, IFNab R2, IGFBP-5, MSP, Nectin-1, Notch-3, NTAL, Neurturin, PRELP, RCOR1, VEGF-D, Cystatin B, Nesfatin, PP and XIAP is indicative of a subject being a non-responder. In some embodiments, an increase in expression of any one of B4GalT1, B7-H2, BMP-9, CCL11, CD97, CHST2, Epo R, FGF-17, FLRT1, IFNab R2, IGFBP-5, MSP, Nectin-1, Notch-3, NTAL, Neurturin, PRELP, RCOR1, VEGF-D and XIAP is indicative of a subject being a responder. In some embodiments, the factor is selected from B4GalT1, B7-H2, BMP-9, CCL11, CD97, CHST2, Epo R, FGF-17, FLRT1, IFNab R2, IGFBP-5, MSP, Nectin-1, Notch-3, NTAL, Neurturin, PRELP, RCOR1, VEGF-D, Cystatin B, Nesfatin, PP and XIAP and an increased expression level of the factor is indicative of the subject being a responder to the immunotherapy. In some embodiments, the factor is selected from B4GalT1, B7-H2, BMP-9, CCL11, CD97, CHST2, Epo R, FGF-17, FLRT1, IFNab R2, IGFBP-5, MSP, Nectin-1, Notch-3, NTAL, Neurturin, PRELP, RCOR1, VEGF-D and XIAP and a lack of increased expression level of the factor is indicative of the subject being a non-responder to the immunotherapy.

In some embodiments, an increase in expression level of the at least one factor or signature indicates the subject is a non-responder. In some embodiments, a lack of increase in expression level of the at least one factor or signature indicates the subject is a non-responder. In some embodiments, a decrease in expression level of the at least one factor or signature indicates the subject is a non-responder. In some embodiments, an increased expression level of the at least one factor or signature indicates the subject is a non-responder. In some embodiments, a decreased expression level of the at least one factor or signature indicates the subject is a non-responder.

In some embodiments, an increase in expression level of the at least one factor or signature indicates the subject is a responder. In some embodiments, a lack of increase in expression level of the at least one factor or signature indicates the subject is a responder. In some embodiments, a decrease in expression level of the at least one factor or signature indicates the subject is a responder. In some embodiments, an increased expression level of the at least one factor or signature indicates the subject is a responder. In some embodiments, a decreased expression level of the at least one factor or signature indicates the subject is a responder.

In some embodiments, a non-responder is a subject that is not responsive to the immunotherapy. In some embodiments, a non-responder is a subject with a non-favorable response to the immunotherapy. In some embodiments, a responder is a subject that is responsive to the immunotherapy. In some embodiments, a responder is a subject with a favorable response to the immunotherapy.

In some embodiments, a signature comprises a plurality of factors and at least one factor of the plurality is increased in non-responders and at least one factor of the plurality of factors is increased in responders. In some embodiments, the signature comprises a combination of a factor increased in responders and a factor increased in non-responders. In some embodiments, a signature comprises a plurality of factors and at least one factor of the plurality is increased in non-responders. In some embodiments, a signature comprises a plurality of factors and at least one factor of the plurality is increased in responders. In some embodiments, a signature comprises a plurality of factors and at least one factor of the plurality is decreased in non-responders. In some embodiments, a signature comprises a plurality of factors and at least one factor of the plurality is decreased in responders. In some embodiments, a signature comprises a plurality of factors and at least one factor of the plurality is not increased in non-responders. In some embodiments, a signature comprises a plurality of factors and at least one factor of the plurality is not increased in responders.

In some embodiments, the increase and/or decrease is from the first determining to the second determining. In some embodiments, the increase and/or decrease is from the expression level in the sample obtained before to the expression level in the sample obtained after. In some embodiments, the increase and/or decrease is an increase/decrease in protein expression. In some embodiments, the increase and/or decrease is an increase/decrease in mRNA expression. In some embodiments, the increase and/or decrease is a significant increase/decrease. In some embodiments, the increase and/or decrease is an increase/decrease of at least one standard deviation. In some embodiments, the increase and/or decrease is an increase/decrease of at least a predetermined amount. In some embodiments, the predetermined amount is a predetermined threshold. In some embodiments, an increase above a predetermined threshold is indicative of a non-responder. In some embodiments, an increase above a predetermined threshold is indicative of a responder. In some embodiments, an increase by more than a predetermined threshold is indicative of a non-responder. In some embodiments, an increase by more than a predetermined threshold is indicative of a responder. In some embodiments, an increase below a predetermined threshold is indicative of a non-responder. In some embodiments, an increase below a predetermined threshold is indicative of a responder. In some embodiments, an increase by less than a predetermined threshold is indicative of a non-responder. In some embodiments, an increase by less than a predetermined threshold is indicative of a responder.

As used herein a “non-favorable response” of the cancer patient indicates “non-responsiveness” of the cancer patient to the treatment with the immunotherapy and thus the treatment of the non-responsive cancer patient with the immunotherapy will not lead to the desired clinical outcome, and potentially to a non-desired outcomes such as tumor expansion, recurrence and metastases. In some embodiments, the method further comprises discontinuing administration of the immunotherapy to a subject that is a non-responder.

In some embodiments, absence of an increase in expression level of the at least one factor or signature indicates the subject is a responder. In some embodiments, absence of an increase in expression level of the at least one factor or signature indicates the subject is a non-responder. In some embodiments, absence of an increased expression level of the at least one factor or signature indicates the subject is a responder. In some embodiments, absence of an increased expression level of the at least one factor or signature indicates the subject is a non-responder. In some embodiments, a responder is a subject that is responsive to the immunotherapy. In some embodiments, a responder is a subject with a favorable response to the immunotherapy. In some embodiments, absence of an increase is a non-significant increase. In some embodiments, absence of an increase is an increase of less than one standard deviation. In some embodiments, absence of an increase is an increase of less than a predetermined amount. In some embodiments, an increase below a predetermined threshold is indicative of a responder. In some embodiments, an increase below a predetermined threshold is indicative of a non-responder. In some embodiments, an increase by less than a predetermined threshold is indicative of a responder. In some embodiments, an increase by less than a predetermined threshold is indicative of a non-responder. In some embodiments, absence of an increase is no increase. In some embodiments, absence of an increase is expression levels that are unchanged. In some embodiments, absence of an increase is the same expression levels at each determination. In some embodiments, absence of an increase is a decrease.

As used herein, a “favorable response” of the cancer patient indicates “responsiveness” of the cancer patient to the treatment with the immunotherapy, namely, the treatment of the responsive cancer patient with the immunotherapy will lead to the desired clinical outcome such as tumor regression, tumor shrinkage or tumor necrosis; an anti-tumor response by the immune system; preventing or delaying tumor recurrence, tumor growth or tumor metastasis. In this case, it is possible and advised to continue the treatment of the responsive cancer patient with the immunotherapy. In some embodiments, the method further comprises continuing to administer the immunotherapy to a subject that is not a non-responder. In some embodiments, a subject that is not a non-responder is a responder.

In some embodiments, the classifier is trained on a training set of expression levels of the at least two factors. In some embodiments, the training set is expression in subjects who are known responders and known non-responders. In some embodiments, the at least two factors are a signature. In some embodiments, the training set is expression of a signature in subjects who are known responders and known non-responders. As used herein, the term “known” refers to subject who have completed a course of the immunotherapy and have been diagnosed as having responded to the therapy or as having not responded to the therapy. In some embodiments, the diagnosis is by a physician. In some embodiments, the change in expression is analyzed with the machine learning classifier. In some embodiments, classifier is trained on the change in expression. In some embodiments, expression levels are changes in expression levels.

In some embodiments, the classifier outputs a response score for the subject. In some embodiments, the classifier outputs a prediction score for the subject. In some embodiments, the classifier outputs a prediction of response or non-response for the subject. In some embodiments, the classifier outputs a confidence interval or the subject's response. In some embodiments, a score below a predetermined threshold indicates the subject is a non-responder. In some embodiments, a score above a predetermined threshold indicates the subject is a non-responder. In some embodiments, a score below a predetermined threshold indicates the subject is a responder. In some embodiments, a score above a predetermined threshold indicates the subject is a responder.

In some embodiments, a trained machine learning model of the present disclosure provides for predicting a response of a patient to the specified treatment or therapy as a binary value, e.g., ‘yes/no,’ ‘responsive/non-responsive,’ or ‘favorable/non-favorable response.’ In some embodiments, the prediction may be expressed on a scale and/or be associated with a confidence parameter. Accordingly, in some embodiments, a machine learning model of the present disclosure may provide for predicting a response rate and/or success rate of a specified treatment in a patient, e.g., the likelihood of a favorable response of a patient to the specified treatment or therapy. Accordingly, in some embodiments, a machine learning model of the present disclosure may provide for predicting a response rate and/or failure rate of a specified treatment in a patient, e.g., the likelihood of a non-favorable response of a patient to the specified treatment or therapy. For example, in some embodiments, the prediction may be expressed in discrete categories and/or on a scale comprising, e.g., ‘complete response,’ ‘partial response,’ ‘stable disease,’ ‘progressive disease,’ ‘pseudo-progression,’ and ‘hyper-progression disease.’ In some embodiments, the prediction may indicate whether a response by a patient is associated with adverse or any other secondary effects, e.g., side-effects. In some embodiments, additional and/or other scales and/or thresholds and/or response criteria may be used, e.g., a gradual scale of 1 (non-responsive) to 5 (responsive).

In some embodiments, one or more annotation schemes may be employed with respect to the training dataset. Accordingly, in some embodiments, a training dataset for a machine learning model of the present disclosure may comprise a plurality of sets of T₁/T₀ ratios or difference in expression values of the factors with respect to at least some of the subjects in the cohort, wherein at least some of these sets of values may be annotated with category labels denoting a response and/or outcome of the treatment in the respective subject. In some embodiments, such annotation may be binary, e.g., positive/negative, and/or expressed in discrete categories, e.g., on a scale of 1-5. In some embodiments, a binary value category label may be expressed, e.g., as ‘yes/no,’ ‘responsive/non-responsive,’ or ‘favorable/non-favorable response.’ In some embodiments, discrete category labels and/or annotations may be expressed on a scale, e.g., ‘complete response,’ ‘partial response,’ ‘stable disease,’ ‘progressive disease,’ ‘pseudo-progression,’ and ‘hyper-progression disease.’ In some embodiments, additional and/or other scales and/or thresholds and/or response criteria may be used, e.g., a gradual scale of 1 (non-responsive) to 5 (responsive). In some embodiments, category labels may be associated with adverse or any other secondary effects or response by a patient, e.g., therapy side-effects.

Machine learning is well known in the art, and any machine learning algorithm known in the art may be used. A skilled artisan will appreciate that by performing the methods of the invention on expression values from subjects with known response/non-response profiles the machine learning algorithm can learn to recognize subjects who will respond/not respond merely based on a small number of factors.

In some embodiments, the method further comprises administering to a subject that is a non-responder an agent that modulates the at least one factor. In some embodiments, the method further comprises administering to a subject that is a non-responder an agent that modulates a pathway that comprises the at least one factor. In some embodiments, modulating the at least one factor is modulating a pathway comprising the at least one factor. In some embodiments, modulating a pathway comprising modulating a driver protein/gene that controls the at least one factor. In some embodiments, modulating a pathway comprising modulating a driver protein/gene that controls the pathway. In some embodiments, modulating a pathway comprising the at least one factor is modulating a receptor of the factor, a ligand or the factor, a paralog of the factor, or a combination thereof. In some embodiments, the modulating is modulating a plurality of factors. In some embodiments, the modulating is modulating a plurality of factors in the signature. In some embodiments, the modulation is modulating each factor in the signature.

In some embodiments, the method further comprises administering to a subject that is a non-responder an agent that modulates a pathway that is differentially expressed in the non-responder. Measuring expression in the subject will provide proteins/RNAs/genes that are increased or decreased in the subject. These proteins/RNAs/genes are considered differentially expressed if they change in a way that is different from in responders. The proteins can be classified into pathways using any pathway analysis tool known in the art. Examples include, but are not limited to, GO analysis, Ingenuity analysis, reactome pathway analysis and STRING functional analysis. In some embodiments, the method further comprises performing pathway analysis on differentially expressed factors. In some embodiments, the method further comprises performing pathway analysis on differentially expressed proteins and/or RNA and/or genes. In some embodiments, the differential expression is in a non-responder as compared to a responder. In some embodiments, the differential expression is in a non-responder as compared to a standard. In some embodiments, the method comprises selecting a pathway. In some embodiments, the selected pathway is a pathway hypothesized to affect non-response to the immunotherapy. In some embodiments, the selected pathway is a pathway hypothesized to cause non-response to the immunotherapy. In some embodiments, the agent inhibits a protein/RNA/gene is the pathway. In some embodiments, the agent activates a protein/RNA/gene in the pathway. In some embodiments, the agent modulates the pathway. In some embodiments, the pathway's activity induces non-response and the agent inhibits the pathway. In some embodiments, the pathway's activity inhibits non-response and the agent activates the pathway. In some embodiments, the agent targets a hub protein/RNA/gene in the pathway. In some embodiments, the agent targets a regulator protein/RNA/gene in the pathway. In some embodiments, the regulator is a master regulator. In some embodiments, the RNA is a regulatory RNA. Examples of regulatory RNAs include microRNAs, long noncoding RNAs, piRNAs and many others.

In some embodiments, the method further comprises administering to a non-responder the agent and the immunotherapy. In some embodiments, the method further comprises continuing to administer the immunotherapy to a non-responder and initiating administration of the agent.

In some embodiments, modulating is inhibiting. In some embodiments, modulating is blocking. In some embodiments, modulating is neutralizing. In some embodiments, modulating is down-regulating. In some embodiments, modulating is reducing. In some embodiments, modulating is degrading. In some embodiments, modulating is rendering inactive. In some embodiments, modulating is decreasing expression. In some embodiments, modulating is administering an antagonist. In some embodiments, the antagonist is an antagonist of the factor. In order to achieve the desired clinical outcome in a non-responder, it may be necessary to blockade an increased factor or a dominant factor controlling the increased factor and then treating the non-responsive cancer patient with a combination of the immunotherapy and a therapeutic agent that blocks the activity of the selected factor. The terms “block”, “blockade”, “neutralize” or “inhibit” or “down regulate” or “reduce” are herein used interchangeably and refer to the capability of an agent of preventing the selected dominant factor from exerting its function/biological activity.

In some embodiments, modulating is activating. In some embodiments, modulating is unblocking. In some embodiments, modulating is enhancing. In some embodiments, modulating is increasing. In some embodiments, modulating is rendering active. In some embodiments, modulating is increasing expression. In some embodiments, modulating is upregulating. In some embodiments, modulating is inducing. In some embodiments, modulating is administering an agonist. In some embodiments, the agonist is an agonist of the factor. In some embodiments, modulating is administering a cofactor. In some embodiments, the cofactor is a cofactor of the factor. In order to achieve the desired clinical outcome in a non-responder, it may be necessary to enhance a not increased factor or a dominant factor controlling the not increased factor and then treating the non-responsive cancer patient with a combination of the immunotherapy and a therapeutic agent that enhances the activity of the selected factor. The terms “activate”, “enhance”, or “increase” or “upregulate” or “induce” are herein used interchangeably and refer to the capability of an agent of enhance the exerted function/biological activity of the selected dominant factor.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal.

The dosage administered will be dependent upon the age, health, and weight of the recipient, kind of concurrent treatment, if any, frequency of treatment, and the nature of the effect desired.

As used herein, the terms “treatment” or “treating” of a disease, disorder, syndrome or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, syndrome or condition, reduce the severity of symptoms associated therewith, or provide improvement to a patient or subject's quality of life.

In some embodiments, administering the agent comprises administering a pharmaceutical composition comprising the agent. In some embodiments, administering the agent comprises administering a therapeutically effective amount of the agent. In some embodiments, a therapeutically effective amount is an amount sufficient to modulate the factor. In some embodiments, the therapeutically effective amount is an amount of an agent effective to treat the cancer in combination with the immunotherapy. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, a pharmaceutical composition comprises the agent and a pharmaceutically acceptable carrier, excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

By another aspect, there is provided a method of converting a non-responder to an immunotherapy to a responder, the method comprising administering to the non-responder an agent that inhibits at least one factor with increased expression in a sample from the non-responder after initiation of the immunotherapy or activates at least one factor with increased expression in a sample from a responder after initiation of the immunotherapy.

In some embodiments, the agent inhibits a factor. In some embodiments, the agent blocks a factor. In some embodiments, the agent decreases a factor. In some embodiments, the agent activates a factor. In some embodiments, the agent increases a factor. In some embodiments, the agent enhances a factor. In some embodiments, the agent modulates a factor.

In some embodiments, a factor with increased expression in a responder is a factor without increased expression in a non-responder. In some embodiments, a factor without increased expression in a non-responder is a factor with increased expression in a responder. It will be understood by a skilled artisan that factors which are increased in non-responders will be inhibited/blocked/down-regulated or otherwise decreased; while factors which are not upregulated or decreased in non-responders will be activated/up-regulated or otherwise increased.

By another aspect, there is provided a kit comprising a reagent adapted to specifically determine the expression level of at least one factor.

In some embodiments, the factor is a factor described hereinabove. In some embodiments, the kit comprises reagents adapted to specifically determine the expression level of a plurality of factors. In some embodiments, the kit comprises reagents adapted to specifically determine the expression level of a signature. In some embodiments, the signature is a signature described hereinabove.

In some embodiments, the expression is selected from protein expression and mRNA expression. In some embodiments, the expression is protein expression. In some embodiments, the expression is mRNA expression. Reagents for detecting protein expression are well known in the art and include antibodies, protein binding arrays, protein binding proteins, and protein binding RNAs. Any reagent capable of binding specifically to the factor can be employed. As used herein, the terms “specific” and “specifically” refer to the ability to quantify the expression of one target to the exclusion of all other targets. Thus, for non-limiting example, an antibody that is specific to a target will bind to that target and no other targets. In some embodiments, the reagent is an antibody. In some embodiments, binding to a target and no other targets is binding measurable to a target and to no other targets. In some embodiments, binding to a target and no other targets is binding significantly to a target and no other targets. Reagents for detecting specific mRNAs are also well known in the art and include, for example, microarrays, primers, hybridization probes, and RNA-binding proteins. Any such reagent may be used. In some embodiments, the reagent is a primer. In some embodiments, the reagent is a pair of primers specific to the factor. It will be understood that a pair of primers that is specific will amplify the target and not significantly or detectably amplify other mRNAs. In some embodiments, the reagent is a nucleic acid molecule. In some embodiments, the reagent is an isolated oligonucleotide. In some embodiments, the isolated oligonucleotide specifically hybridizes to the factor or an mRNA of the factor. In some embodiments, the isolated oligonucleotide is no longer than 15, 20, 25, 30, 35, 40, 45 or 50 nucleotides in length. Each possibility represents a separate embodiment of the invention. In some embodiments, the isolated oligonucleotide hybridizes to only a portion of an mRNA of the factor. In some embodiments, the isolated oligonucleotide hybridizes to an mRNA of the factor with 100% complementarity. In some embodiments, the isolated oligonucleotide hybridizes to an mRNA of the factor with at least 90% complementarity. In some embodiments, the isolated oligonucleotide hybridizes to an mRNA of the factor with at least 95% complementarity. In some embodiments, the isolated oligonucleotide does not hybridize to an mRNA of a gene other than the factor with a complementarity of greater than 70, 75, 80, 85, 90, 95, 97, 99 or 100%. Each possibility represents a separate embodiment of the invention. In some embodiments, the isolated oligonucleotide does not hybridize to an mRNA of a gene other than the factor with 100% complementarity.

In some embodiments, the kit further comprises at least one reagent adapted to specifically determine the expression level of a control. In some embodiments, the control is a control such as described hereinabove. It will be understood that if the kit comprises reagents for determining protein expression of the factor, then the reagent for determining expression of the control would also determine protein expression. Similarly, for mRNA expression the reagents for the control would match the reagents for the factor. In some embodiments, the reagent for determining expression of the factor and the reagent for determining expression of the control are the same type of reagent.

In some embodiments, the kit further comprises detectable tags or labels. In some embodiments, the reagents are hybridized or attached to the labels. In some embodiments, the tag or label is a nucleic acid tag or label. In some embodiments, the nucleic acid tag or label is a primer. In some embodiments, the kit further comprises a secondary reagent for detection of the specific reagents. In some embodiments, the secondary reagents are non-specific and will detect all or a subset of the specific reagents. In some embodiments, the secondary reagents are secondary antibodies. In some embodiments, the secondary reagents are detectable. In some embodiments, the secondary reagents comprise a tag or label. In some embodiments, the tag or label is detectable. In some embodiments, a detectable molecule comprises a detectable moiety. Examples of detectable moieties include fluorescent moieties, dyes, bulky groups and radioactive moieties. In some embodiments, the kit further comprises a solution for rendering a protein susceptible to binding. In some embodiments, the kit further comprises a solution for rendering a nucleic acid susceptible to hybridization. In some embodiments, the nucleic acid is an mRNA. In some embodiments, the kit further comprises a solution for lysing cells. In some embodiments, the kit further comprises a solution for isolating plasma from blood. In some embodiments, the kit further comprises a solution for purification of proteins. In some embodiments, the kit further comprises a solution for purification of nucleic acids.

In some embodiments, a reagent is attached or linked to a solid support. In some embodiments, the reagent is non-natural. In some embodiments, the reagent is artificial. In some embodiments, the reagent is in a non-organic solution. In some embodiments, the reagent is ex vivo. In some embodiments, the reagent is in a vial. In some embodiments, the solid support is non-organic. In some embodiments, the solid support is artificial. In some embodiments, the solid support is an array. In some embodiments, the solid support is a chip. In some embodiments, the solid support is a bead.

In some embodiments, the kit comprises reagents. In some embodiments, the kit comprises a plurality of reagents. In some embodiments, the reagents are for determining expression levels of at least two factors. In some embodiments, the reagents are for determining expression levels of a plurality of factors. In some embodiments, a plurality is at least 2, 3, 4, 5, 6, 7, 8, 9, 10, 11, 12, 13, 14, 15, 16, 17, 18, 19, 20, 21, 22, 23, 24, or 25 factors. Each possibility represents a separate embodiment of the invention. In some embodiments, the plurality of factors is for detecting a signature. Thus, it will be understood that if the signature comprises a given number of factors, then the kit will have at least that number of reagents specifically adapted to determine the expression of those factors. In some embodiments, the minimum number of factors is the minimum number of factors in the signature.

In some embodiments, the kit comprises at most 10, 15, 20, 25, 30, 35, 40, 45, 50, 60, 70, 75, 80, 90 or 100 reagents. Each possibility represents a separate embodiment of the invention. In some embodiments, reagents are different reagents. In some embodiments, the kit comprises at most 10 reagents. In some embodiments, the kit comprises at most 25 reagents. In some embodiments, the kit comprises at most 50 reagents. In some embodiments, the kit comprises the number of reagents needed to detect a signature. In some embodiments, a kit consists of the reagents for detecting a signature. In some embodiments, the kit is limited to the reagents for detecting a signature. In some embodiments, the reagents consist of the reagents for detecting a signature. In some embodiments, the kit may comprise other elements, but the reagents consist of the number of reagents needed to detect a signature. Thus, for example, if a signature consisted of uPAR, P-Cadherin, IL-6, BMP-4, PRELP and OPN then a kit may consist of reagents specific for detection of uPAR, P-Cadherin, IL-6, BMP-4, PRELP and OPN or the reagents of the kit may consist of reagents specific for the detection of uPAR, P-Cadherin, IL-6, BMP-4, PRELP and OPN.

By another aspect there is provided, a computer program product comprising a non-transitory computer-readable storage medium having program code embodied thereon, the program code executable by at least one hardware processor to:
a. receive an expression level of at least two factors in a sample obtained from a subject suffering from lung cancer at a first time point relative to the initiation of immunotherapy;
b. receive an expression level of the at least two factors in a sample obtained from the subject at a second time point relative to the initiation of immunotherapy;
c. analyze the determined expression levels with a machine learning classifier; and
d. output for the subject a response score to the immunotherapy.

In some embodiments, the computer program product comprising a non-transitory computer-readable storage medium having program code embodied thereon, the program code executable by at least one hardware processor to:
a. receive an expression level of at least two factors in a sample obtained from a subject suffering from lung cancer before initiation of an immunotherapy;
b. receive an expression level of the at least two factors in a sample obtained from the subject after initiation of the immunotherapy;
c. analyze the determined expression levels with a machine learning classifier; and
d. output for the subject a response score to the immunotherapy.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, R, Python, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

As used herein, the term “about” when combined with a value refers to plus and minus 10% of the reference value. For example, a length of about 1000 nanometers (nm) refers to a length of 1000 nm+/−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Cohort Description and Data Preparation for Analysis

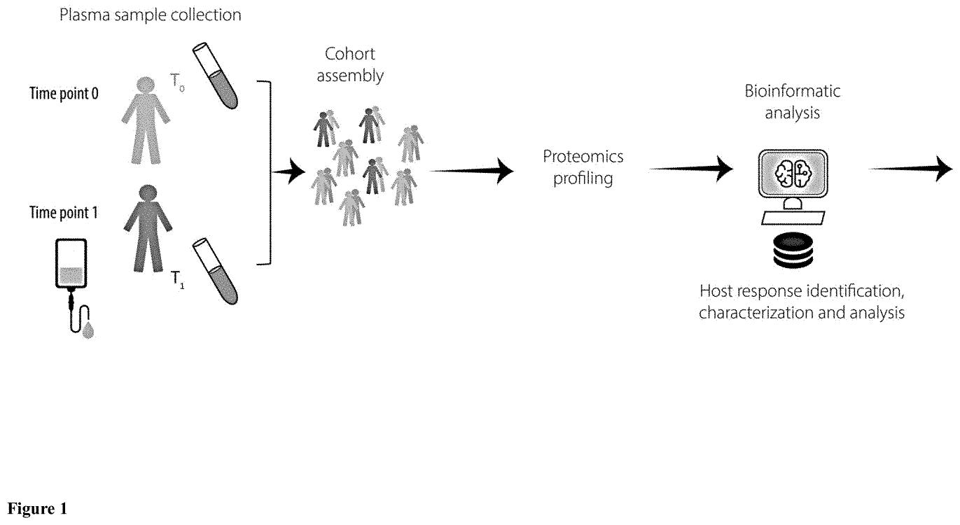

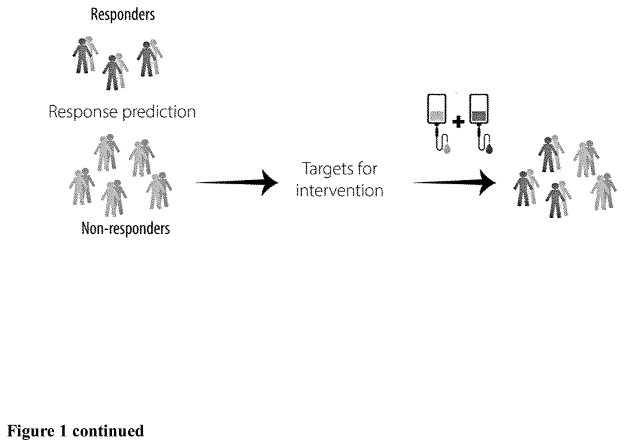

To generate a classifier that would enable prediction of response to treatment based on host-response data, the changes between TO and T1 were determined. For each patient, plasma samples pre- (T0) and early on- (T1) treatment were collected (FIG. 1), and the proteomic changes during anti-PD1/PD-L1 treatment were profiled using an antibody array (RayBiotech). A total of 1000 proteins were evaluated per sample. The response to treatment was determined using RECIST or clinical benefit estimation.

The cohort used for classification comprised plasma samples from 52 NSCLC patients (Cohort A) that received various immunotherapy treatment regimens including Pembrolizumab, Pembrolizumab in combination with chemotherapy, Nivolumab or another modality that involved mostly Atezolizumab. Out of the 52 samples, 30 patients were defined as responders (R), and 22 were defined as non-responders (NR).

The protein expression data was first subjected to quality check and normalization and following this step classification was performed.

Preparation of the data for analysis was done by applying the following steps in the following order:
1. Measurements at T0 or T1 that were below the limit of detection (LOD) were rounded to the LOD.
2. log 2(T1/T0)—The log of the fold-change between T1 and TO was calculated and further used for any downstream analysis.
3. Normalization—For each protein, the median of the log fold-change over all samples was subtracted from all measurements. This step was crucial for SEMMS to converge.
4. Proteins with missing or 0 values in more than 50% of the samples were excluded; following this filtration approach, the remaining 863 proteins were subjected to the following classification.

### Example 2: Classifier Training Process and Feature Selection

The entire analysis was performed using R software, and was divided into two steps:
1. Feature (protein) selection—assuming that only a small (but unknown) fraction of the candidate proteins explains the response to treatment, a feature selection method was employed to identify candidate proteins among the high number of proteins in the dataset, that are relevant for response prediction. For this purpose, two methods were used: SEMMS, a method for identifying significant predictors for a variable, and L2N, a method for identifying differentially expressed factors.
2. Supervised learning—to classify each subject into one of the two predicted classes: responders and non-responders, linear Support Vector Machine (linear SVM) was used, though Generalized Linear Model (GLM) and Random Forest (RF) are also applicable.

In order to identify proteins that are stable in response prediction, each algorithm was repeated 300 times, each time with a random subset of 75% of the subjects. Each subset was balanced between the responder and non-responder group (specifically, each subset contained 22 of the 30 responders and 16 of the 22 non responders). Every repeat that successfully converged and yielded a model with area under the curve (AUC) of the receiver operating characteristics (ROC) plot >0.5 was included in the analysis.

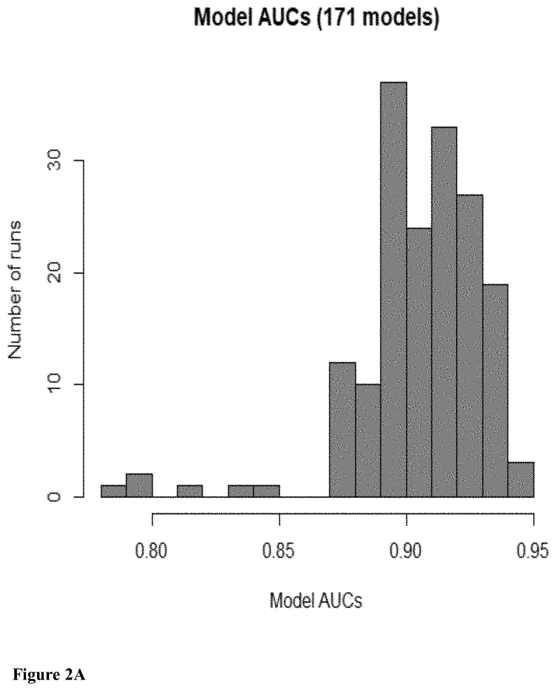

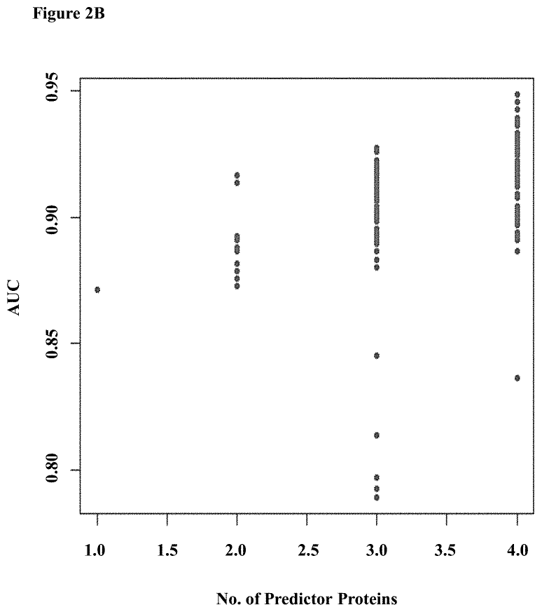

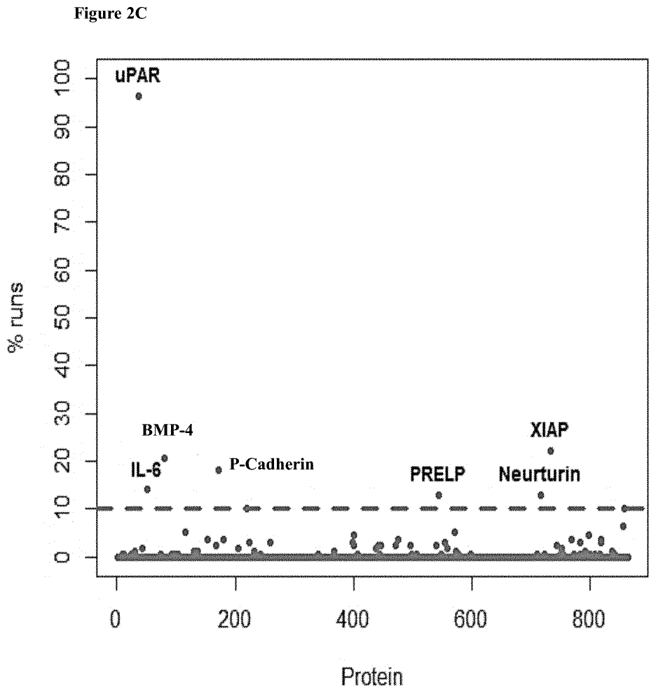

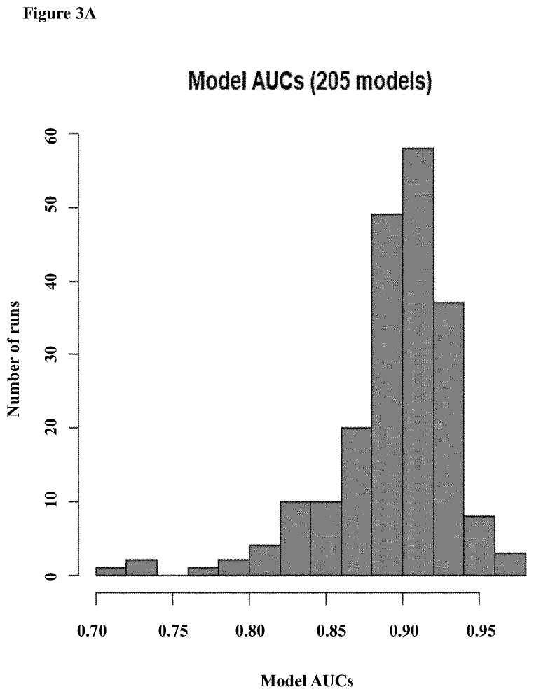

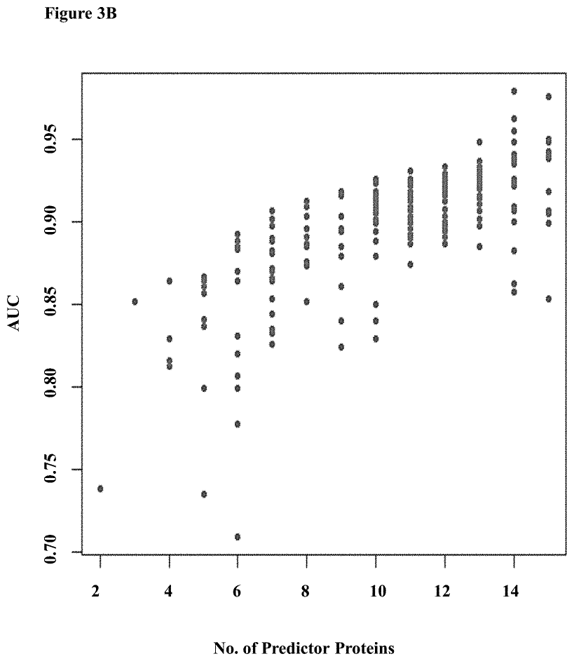

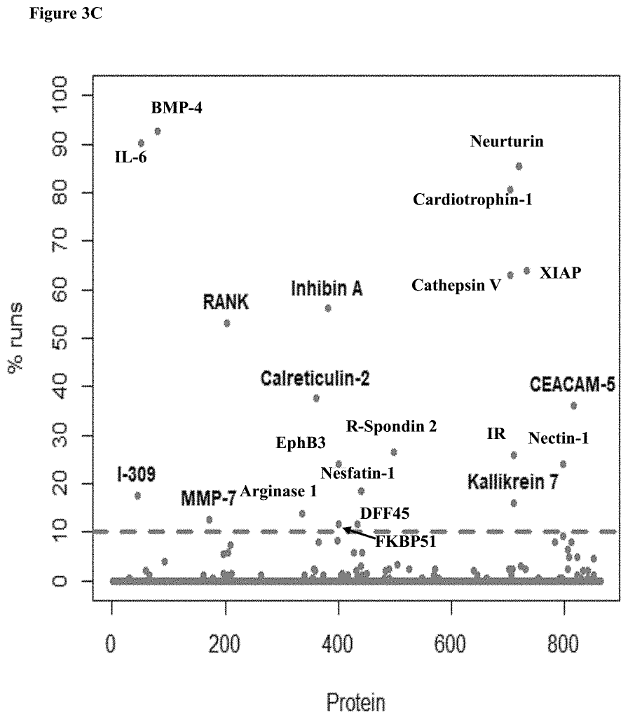

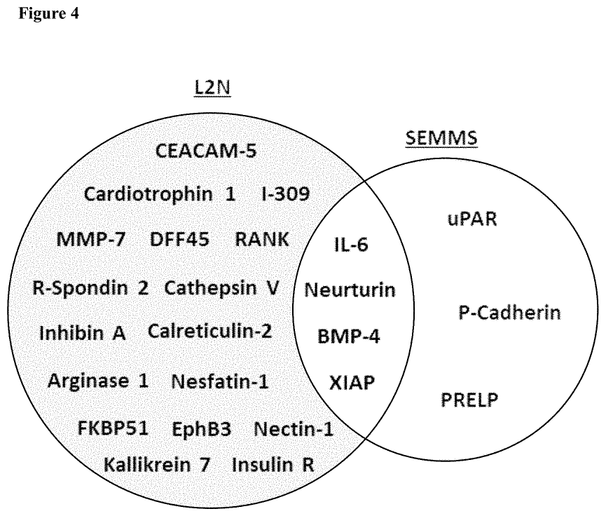

When running SEMMS (FIG. 2) the protein uPAR (PLAUR) emerged as a highly stable protein appearing in 96% of the models. No other protein crossed a 25% threshold. When using a 10% threshold (The % of recurrence of a certain protein in the obtained models) the proteins IL-6, BMP-4, P-Cadherin, PRELP, Neurturin and XIAP emerged. When running L2N analysis (FIG. 3), one can see that overall more proteins were stable in a higher percentage of the models and the models were larger compared to the SEMMS results. The proteins IL-6, BMP-4, Neurturin, and Cardiotrophin 1 emerged as highly stable proteins, they appeared in >80% of the models. The proteins 1-309, MMP-7, RANK, Arginase 1, Calreticulin-2, Inhibin A, EphB3, FKBP51 DFF45, Nesfatin-1, R-Spondin 2, Cathepsin V, Insulin R, Kallikrein 7, XIAP, and CEACAM-5 appeared in >10% of the models. Proteins common to both SEMMS and L2N analyses are presented in FIG. 4 and are summarized in Table 2.

TABLE 2
Top proteins identified by SEMMS and L2N algorithms.

| SEMMS | | L2N | |
|---|---|---|---|
| Protein | % of models | Protein | % of models |
| uPAR | 96.49123 | BMP-4 | 92.68293 |
| XIAP | 22.22222 | IL-6 | 90.2439 |
| BMP-4 | 20.46784 | Neurturin | 85.36585 |
| P-Cadherin | 18.12866 | Cardiotrophin-1 | 80.48781 |
| IL-6 | 14.03509 | XIAP | 63.90244 |
| PRELP | 12.8655 | Cathepsin V | 62.92683 |
| Neurturin | 12.8655 | Inhibin A | 56.09756 |
| BMP-9 | 9.94152 | RANK | 53.17073 |
| OPN | 9.94152 | Calreticulin-2 | 37.56098 |
| MSP | 6.432749 | CEACAM-5 | 36.09756 |
| VEGF-D | 5.263158 | R-Spondin 2 | 26.34146 |
| CK18 | 5.263158 | Insulin R | 25.85366 |
| EphB3 | 4.678363 | EphB3 | 23.90244 |
| Nectin-1 | 4.678363 | Nectin-1 | 23.90244 |
| CA9 | 3.508772 | Nesfatin-1 | 18.04878 |
| CA19-9 | 3.508772 | I-309 | 17.56098 |
| Semaphorin 4D | 3.508772 | Kallikrein 7 | 16.09756 |
| B7-H2 | 3.508772 | Arginase 1 | 13.65854 |
| Cystatin E M | 3.508772 | MMP-7 | 12.68293 |
| CD97 | 2.923977 | FKBP51 | 11.70732 |
| CA125 | 2.923977 | DFF45 | 11.70732 |
| Draxin | 2.923977 | OPN | 9.94152 |
| B4GalTl | 2.923977 | NKp30 | 9.268293 |
| FGF-17 | 2.923977 | Draxin | 8.292683 |
| Cystatin B | 2.923977 | Cerberus 1 | 7.804878 |
| IL-18 | 2.339181 | FGF-17 | 7.804878 |
| FKBP51 | 2.339181 | Cadherin-13 | 7.804878 |
| PRX2 | 2.339181 | TRANCE | 7.317073 |
| Serpin B8 | 2.339181 | Semaphorin 7A | 6.341463 |
| NPDC-1 | 2.339181 | TACI | 5.853659 |
| OSCAR | 2.339181 | Cortactin | 5.853659 |
| PILR-alpha | 2.339181 | PP | 5.853659 |
| Ephrin-A4 | 2.339181 | HAI-2 | 5.365854 |

### Example 3: Single Protein Predictions Using Linear SVM

Following the feature selection using the 10% threshold, the Support Vector Machine (SVM) algorithm was used for discovering a predictive signature for response to treatment based on host-response. Linear SVM was employed and cross-validation was performed using 4-fold validation and a single prediction was generated for each subject. Briefly, a model was trained based on 75% of the samples in the cohort (training set) and then its success was tested on the remaining 25% of the samples (test set) to see that it generalizes well to new data. This process was repeated 4 times (hence 4-fold cross-validation), once for every 25% of the data as the test set. This way four different models were generated, each trained on 75% of the data and tested on 25% of the data. Using this method, 4 test sets were obtained that overall contain all the samples in the cohort.

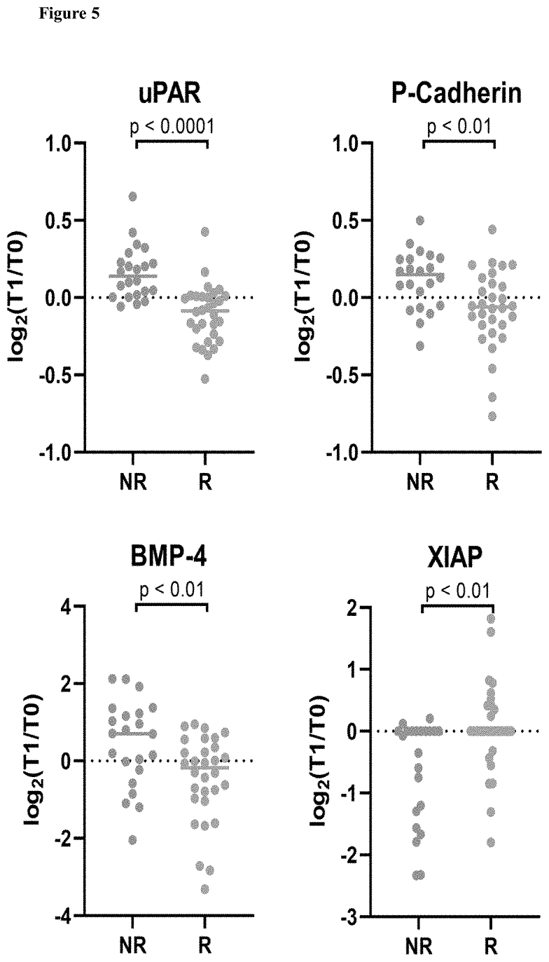

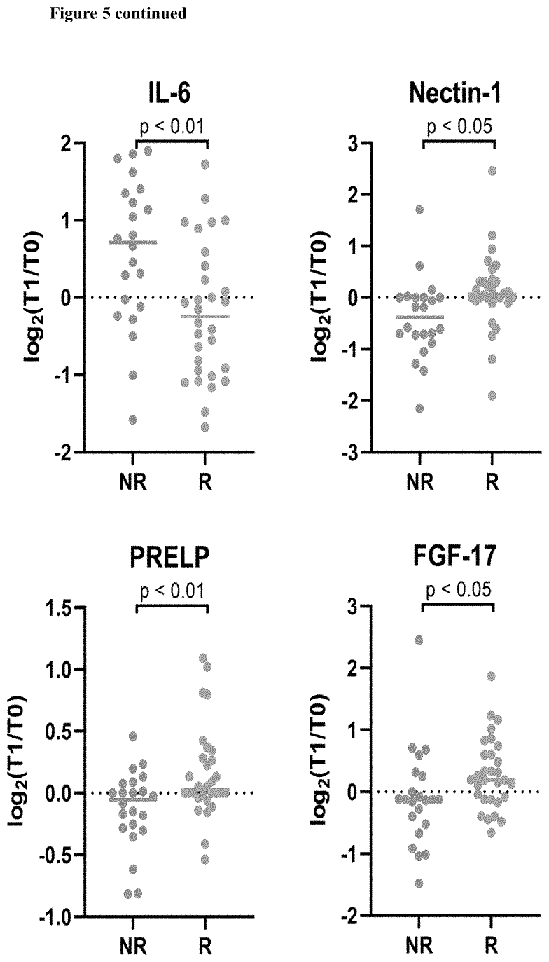

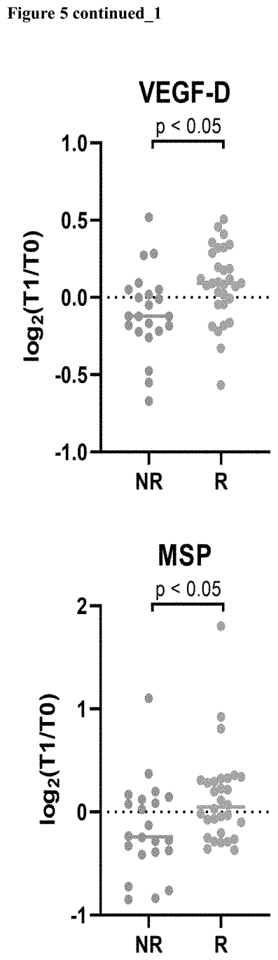

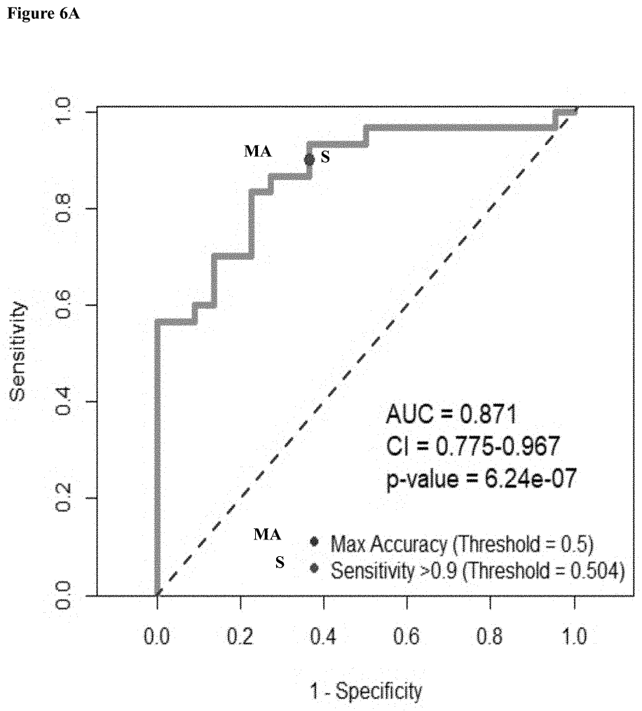

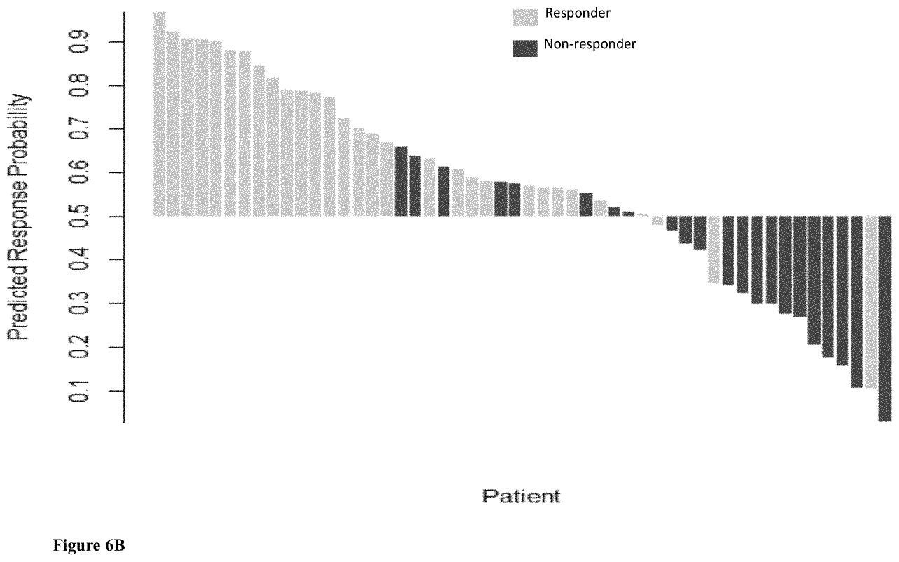

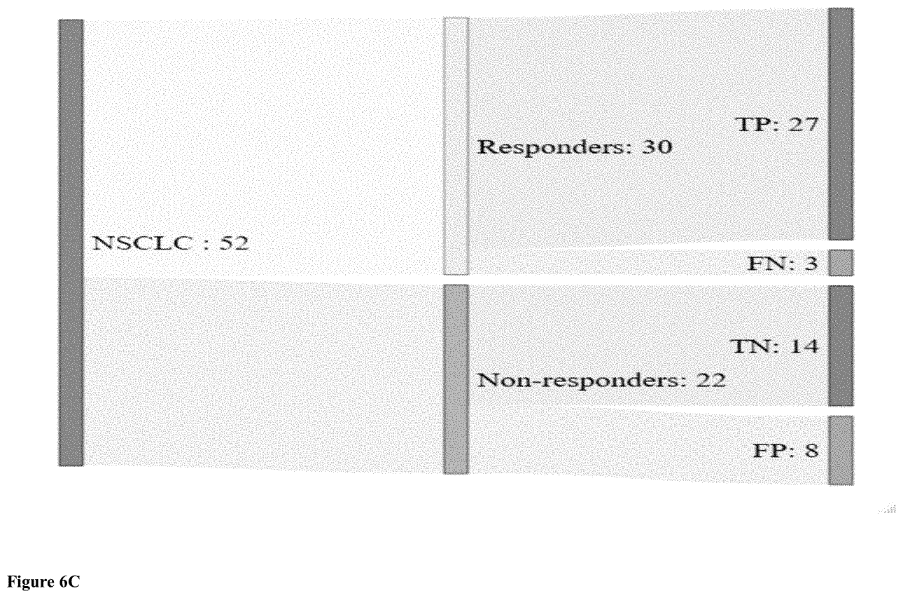

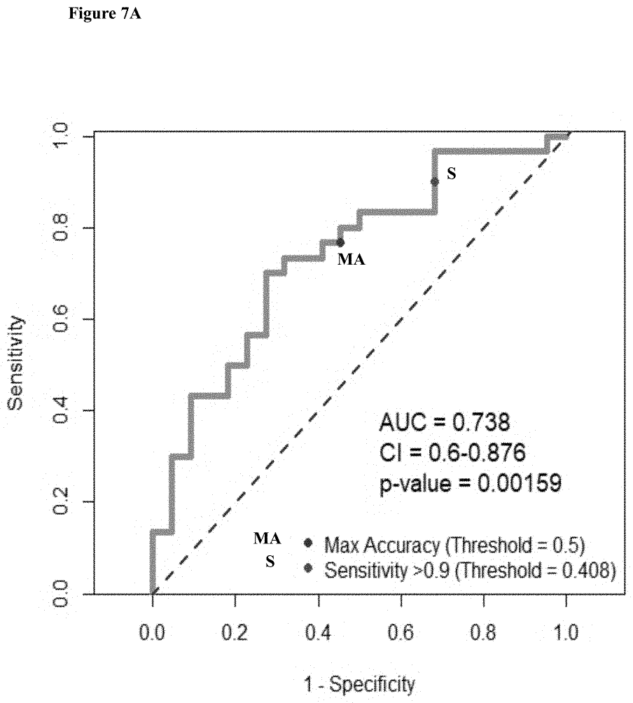

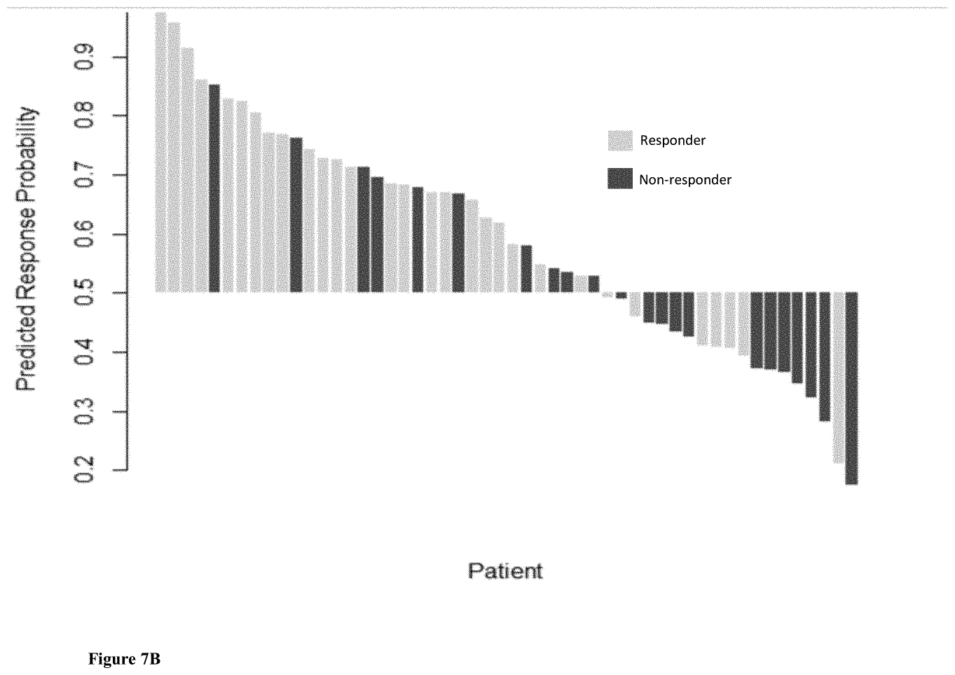

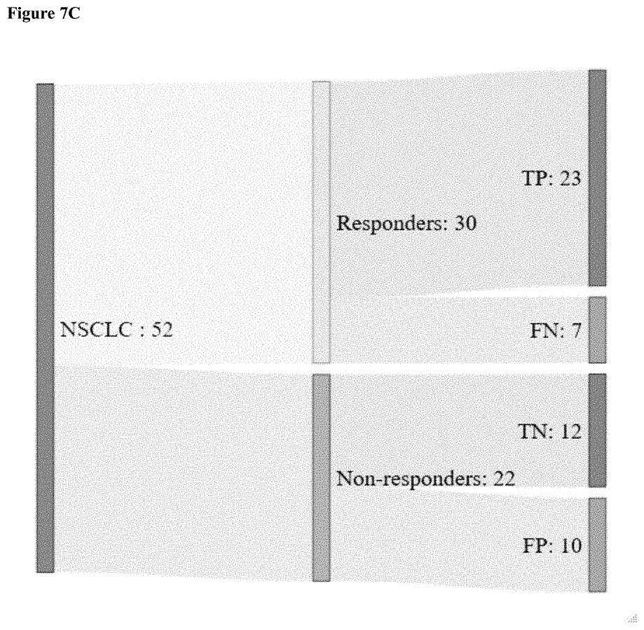

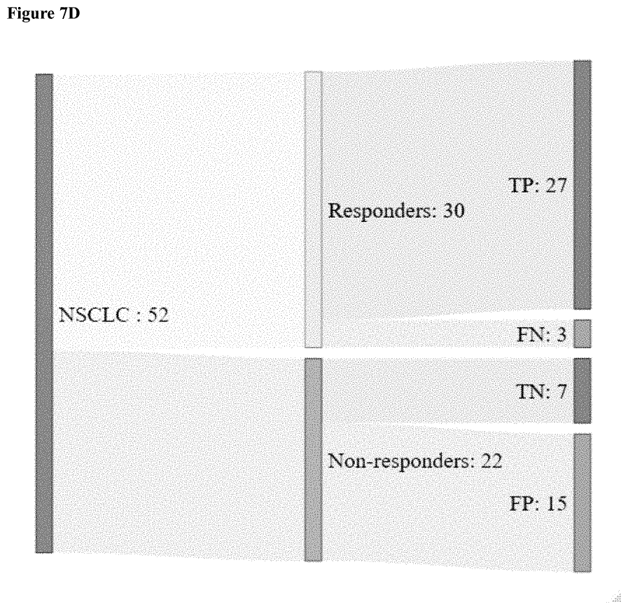

The top 20 protein predictors obtained using this approach show a varied AUC between 0.871 and 0.569 (Table 3); all of the top 10 proteins had p-values below 0.05 (FIG. 5). The best single protein predictors were uPAR and P-Cadherin (FIG. 6 and FIG. 7, respectively).

TABLE 3
ROC AUC of the top 20 single protein
signatures obtained using linear SVM.

| Proteins | AUC |
|---|---|
| uPAR | 0.871 |
| P-Cadherin | 0.738 |
| IL-6 | 0.735 |
| Nectin-1 | 0.735 |
| VEGF-D | 0.721 |
| BMP-4 | 0.720 |
| XIAP | 0.713 |
| PRELP | 0.702 |
| FGF-17 | 0.698 |
| MSP | 0.689 |
| EphB3 | 0.668 |
| OPN | 0.662 |
| IL-18 | 0.674 |
| Insulin R | 0.634 |
| Neurturin | 0.633 |
| Inhibin A | 0.632 |
| Cathepsin V | 0.632 |
| MMP-7 | 0.605 |
| Calreticulin-2 | 0.592 |
| RANK | 0.569 |

This approach was based on a list of proteins from which proteins with missing or 0 values in more than 50% of the samples were excluded at step 4 of the data preparation for analysis. To inspect if among the proteins that were filtered out due to low measurability there are some protein predictors (and with the option of more sensitive assays will be potential good predictors, or that they are relevant for a defined subgroup of the entire cohort), the measurability threshold was removed (the step of filtering out of proteins that were below the LOD in both TO and T1 for more than 50% of subjects), and proteins that had either TO or T1 above the LOD were used for the analysis. Using this approach three additional significant predictors were obtained: IL-2, Epo R and TAFA5 (Table 4).

TABLE 4
ROC AUC of single protein signatures obtained using linear
SVM on protein candidates with low measurability.

| | | % of Subjects Having Either T0 or T1 |
|---|---|---|
| Protein | AUC | above LOD |
| IL-2 | 0.712 | 0.44 |
| Epo R | 0.669 | 0.42 |
| TAFA5 | 0.653 | 0.40 |
| KIR2DL3 | 0.627 | 0.38 |
| BMPR-IB | 0.623 | 0.35 |
| Semaphorin 6C | 0.608 | 0.15 |
| LEDGF | 0.608 | 0.33 |
| IL-5 | 0.605 | 0.33 |
| Testican 2 | 0.604 | 0.31 |
| IL-5 Ra | 0.600 | 0.19 |
| TAFA1 | 0.600 | 0.37 |

A parallel method for identification of protein predictors is using linear SVM without a prior feature selection step. For this purpose, the AUC of the linear SVM model built as described above was computed for each of the 1000 determined proteins. Proteins with AUC>0.65 were defined as highly predictive (See Table 5; 35 of 1000 proteins, 3.5%). This threshold roughly correlated with significant AUCs (p<0.05). Proteins with an AUC between 0.58 and 0.65 were defined as moderately predictive (141 of 1000 proteins, 14.1%). The remaining 821 proteins were considered lowly predictive or non-predictive or had very low expression.

TABLE 5
ROC AUC of single protein signatures obtained using linear SVM

| | | Increased |
|---|---|---|
| Protein | AUC | expression in |
| uPAR | 0.871 | Non-Responders |
| P-Cadherin | 0.738 | Non-Responders |
| IL-6 | 0.735 | Non-Responders |
| Nectin-1 | 0.735 | Responders |
| VEGF-D | 0.721 | Responders |
| BMP-4 | 0.720 | Non-Responders |
| XIAP | 0.713 | Responders |
| PRELP | 0.702 | Responders |
| FGF-17 | 0.698 | Responders |
| CA125 | 0.695 | Non-Responders |
| Periostin | 0.694 | Non-Responders |
| MSP | 0.689 | Responders |
| CK18 | 0.683 | Non-Responders |
| Ephrin-A4 | 0.682 | Non-Responders |
| IL-4 Ra | 0.677 | Non-Responders |
| Granulysin | 0.676 | Non-Responders |
| IL-18 | 0.674 | Non-Responders |
| B4GalTl | 0.673 | Responders |
| BMP-9 | 0.670 | Responders |
| Semaphorin 4D | 0.670 | Non-Responders |
| EphB3 | 0.668 | Non-Responders |
| HGF | 0.665 | Non-Responders |
| IFNab R2 | 0.663 | Responders |
| OPN | 0.662 | Non-Responders |
| FLRT1 | 0.660 | Responders |
| B7-H2 | 0.659 | Responders |
| Notch-3 | 0.659 | Responders |
| CHST2 | 0.658 | Responders |
| I-309 | 0.658 | Non-Responders |
| CD97 | 0.656 | Responders |
| NTAL | 0.653 | Responders |
| Draxin | 0.653 | Non-Responders |
| IGFBP-4 | 0.653 | Non-Responders |
| IGFBP-5 | 0.652 | Responders |
| RCOR1 | 0.650 | Responders |
| Cystatin B | 0.64098 | Responders |
| Nesfatin | 0.62099 | Responders |
| PP | 0.61737 | Responders |
| DFF45 | 0.60077 | Non-Responders |

### Example 4: Multi-Protein Predictions Using Linear SVM

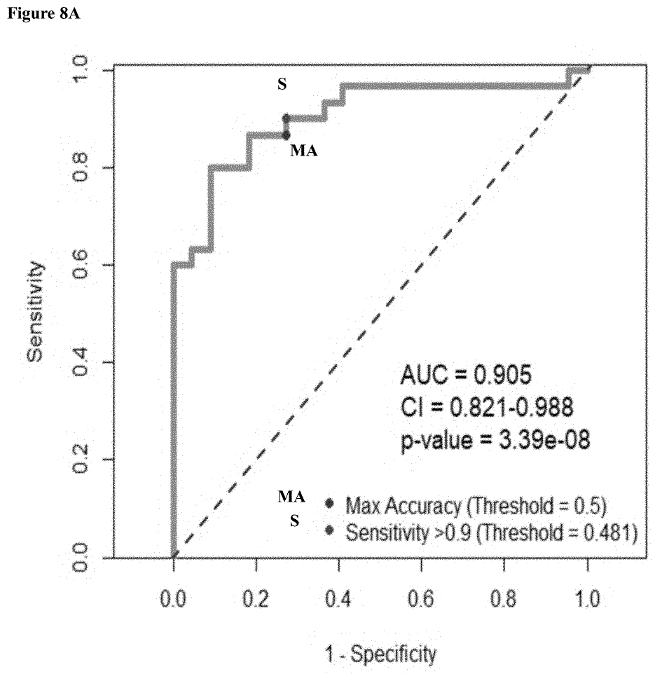

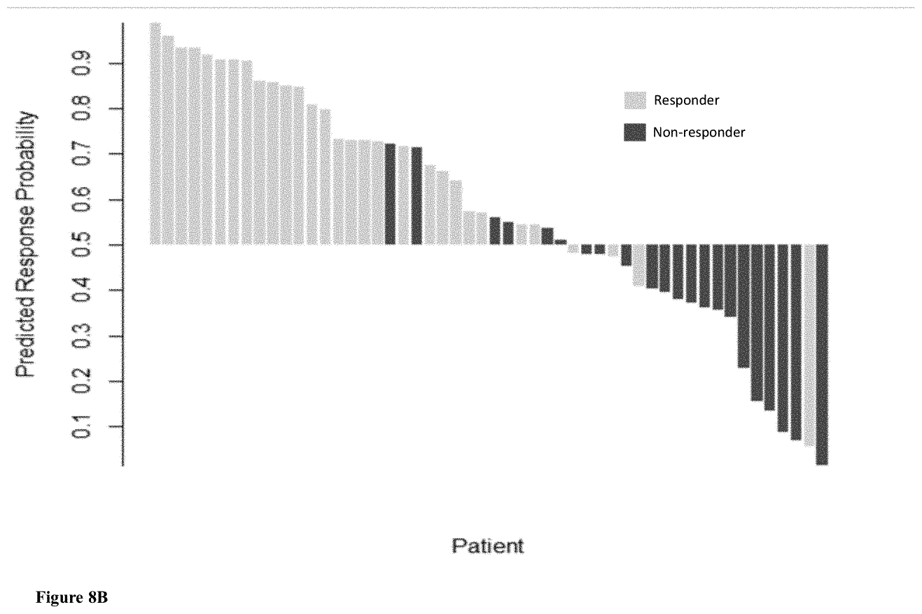

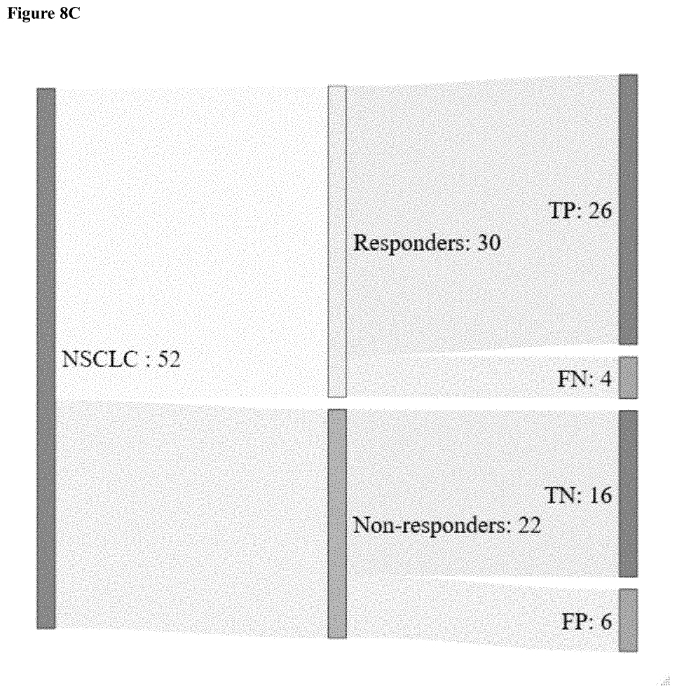

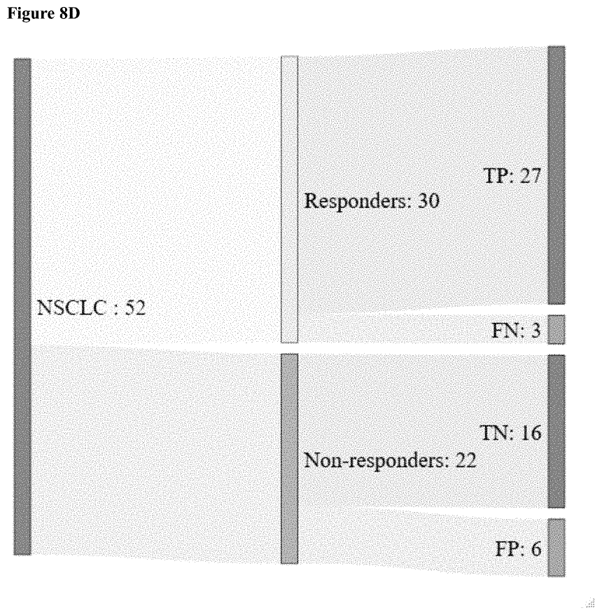

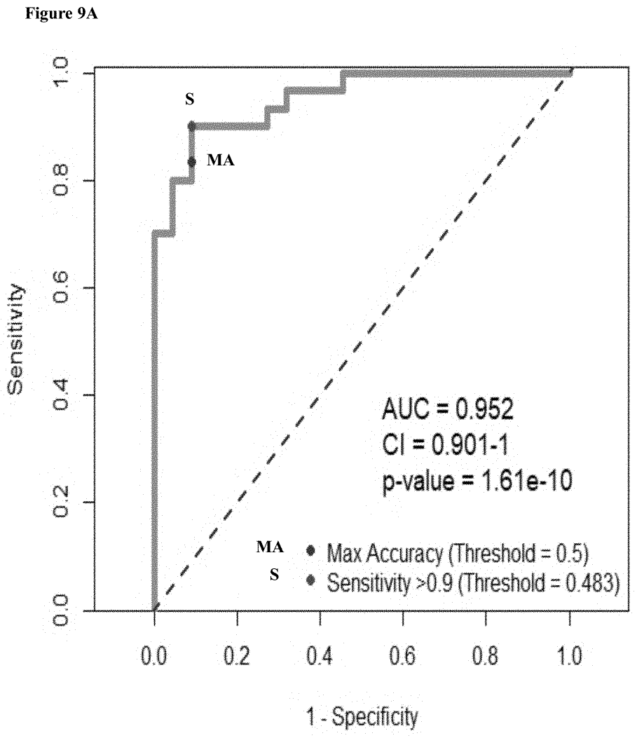

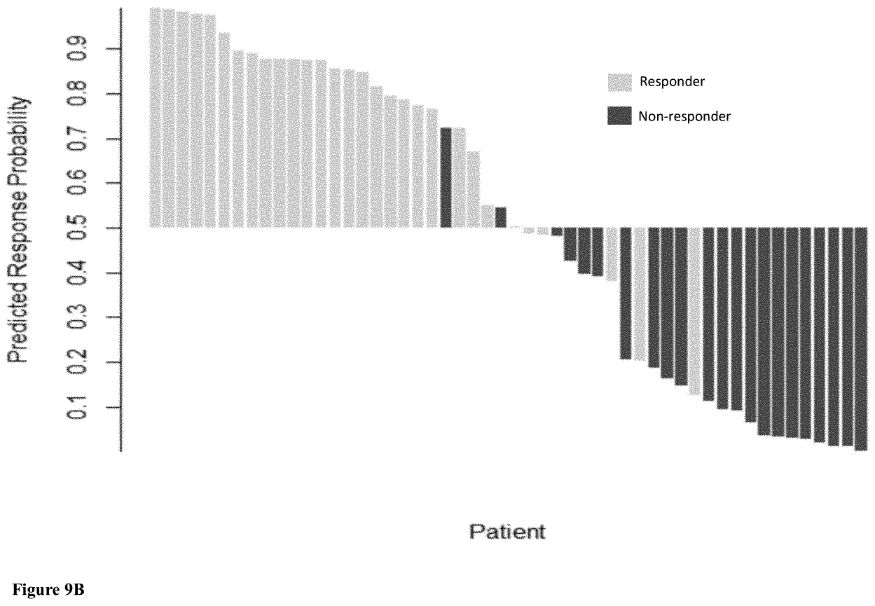

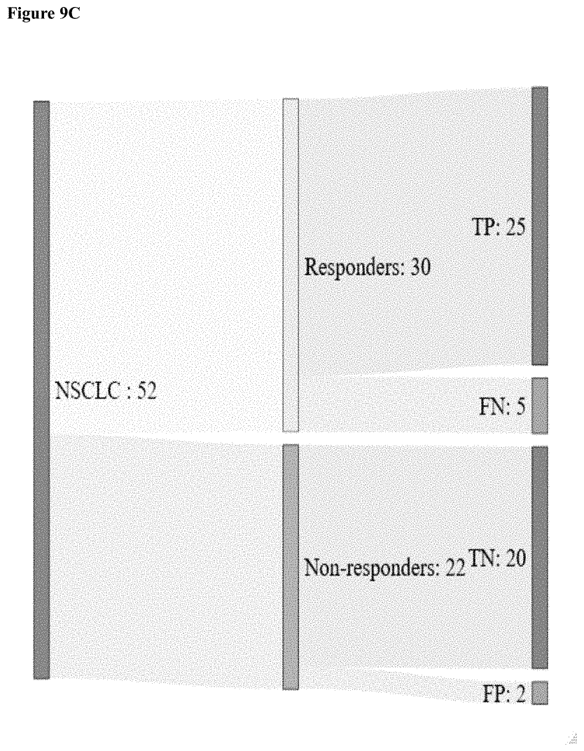

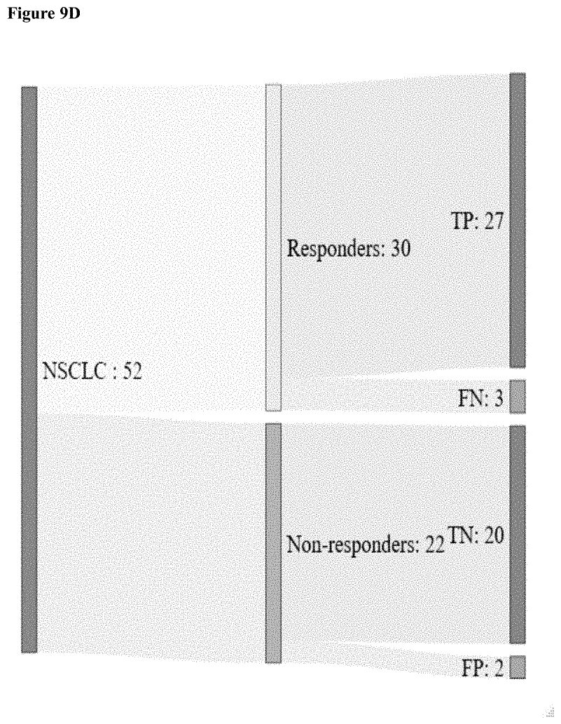

Following the single protein predictions, combinations of multiple proteins from the list presented in Table 5 were used as predictors. These models were generated to maximize prediction ROC AUC with a minimal number of proteins. The best prediction using 2 proteins was achieved using uPAR and IL-6, yielding an AUC of 0.905 (FIG. 8). Using uPAR, IL-6, PRELP, XIAP, and P-Cadherin yielded an AUC of 0.952 (FIG. 9). Additional models based on stable proteins yielding high ROC AUC are listed in Table 6.

TABLE 6
ROC AUC of multi-protein signatures using linear SVM.

| Proteins | AUC |
|---|---|
| uPAR, IL-6, BMP-4, P-Cadherin, PRELP, XIAP, OPN | 0.965 |
| uPAR, IL-6, PRELP, XIAP, P-Cadherin | 0.952 |
| IL-6, BMP-4, P-Cadherin, PRELP, Neurturin, XIAP, OPN | 0.947 |
| uPAR, PRELP, XIAP, IL-6 | 0.938 |
| uPAR, PRELP, XIAP | 0.911 |
| uPAR, IL-6 | 0.905 |
| P-Cadherin, PRELP, XIAP | 0.903 |
| uPAR, IL-6, BMP-4 | 0.892 |
| uPAR, XIAP | 0.885 |
| P-Cadherin, PRELP | 0.858 |
| PRELP, XIAP | 0.858 |
| IL-6, XIAP | 0.833 |
| IL-6, Neurturin | 0.821 |
| P-Cadherin, IL-18, FGF-17 | 0.817 |
| IL-6, Nectin-1 | 0.814 |
| P-Cadherin, Nectin-1 | 0.809 |
| PRELP, IL-6 | 0.800 |

### Example 5: Classifier Validation

To validate the classifiers, a second set of data (cohort B), a cohort comprised of 82 advanced stage NSCLC patients treated with anti-PD1 (either Nivolumab or Pembrolizumab) was assembled; The response to treatment was determined either using response evaluation criteria in solid tumors (RECIST) 1.1 or estimated based on clinical evaluation. For each patient, plasma samples pre- (T0) and early on- (T1) treatment were collected, and the proteomic changes following anti-PD1 treatment was determined. T0 and T1 values below the limit of detection (LOD) were rounded to LOD and the T1/T0 ratios (fold change) for each protein following log 2 transformation were calculated. Next, proteins with less than 50% measurability were filtered out and data was normalized.

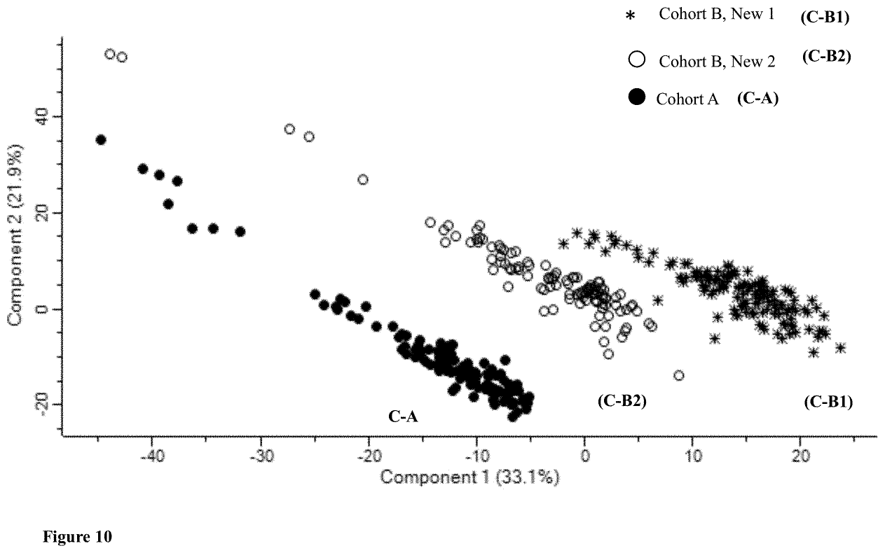

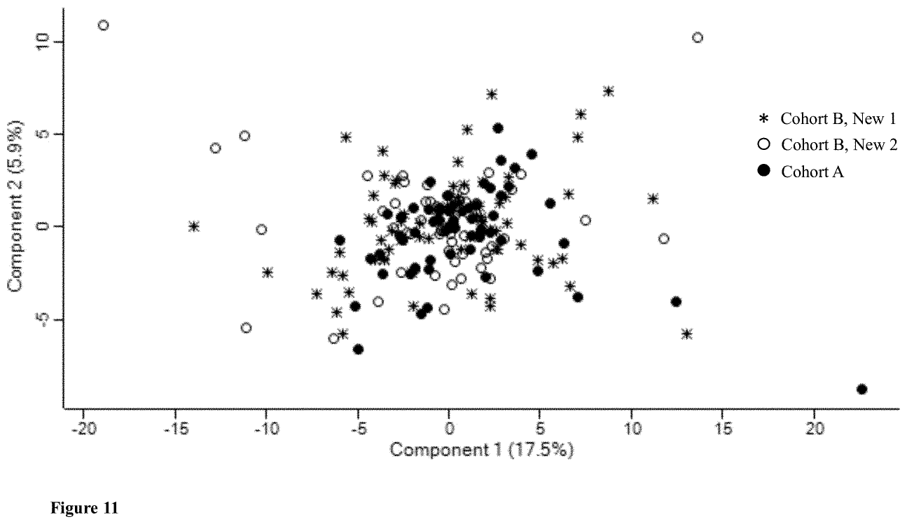

Cohort B was based on two sets that were protein profiled at two different time periods and thus were named New 1 and New 2. Following data normalization and quality control, checks were performed to identify technical biases and technical outliers (no outliers were removed in this analysis). The batch effects between all three batches (Cohort A, Cohort B new 1 and Cohort B new 2) were also analyzed. Principal component analysis (PCA) showed that while for the T0 and T1 data there are 3 clear clusters reflecting the three datasets (Cohort A and Cohort B New 1 and New 2 datasets; FIG. 10), the T1/T0 data showed a single cluster comprised of all 3 batches, without any separation between the batches based on the first or the second component (FIG. 11). Altogether, this indicates that at the proteome level, there are no batch effects in the T1/T0 data.

### Example 6: Dataset-Level QC and Validation of Predictive Proteins

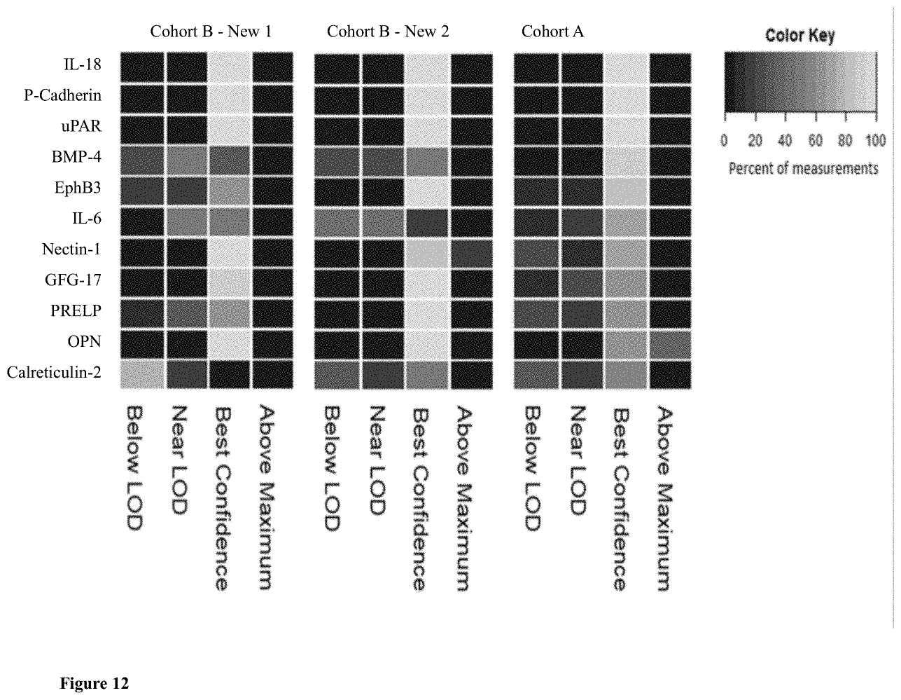

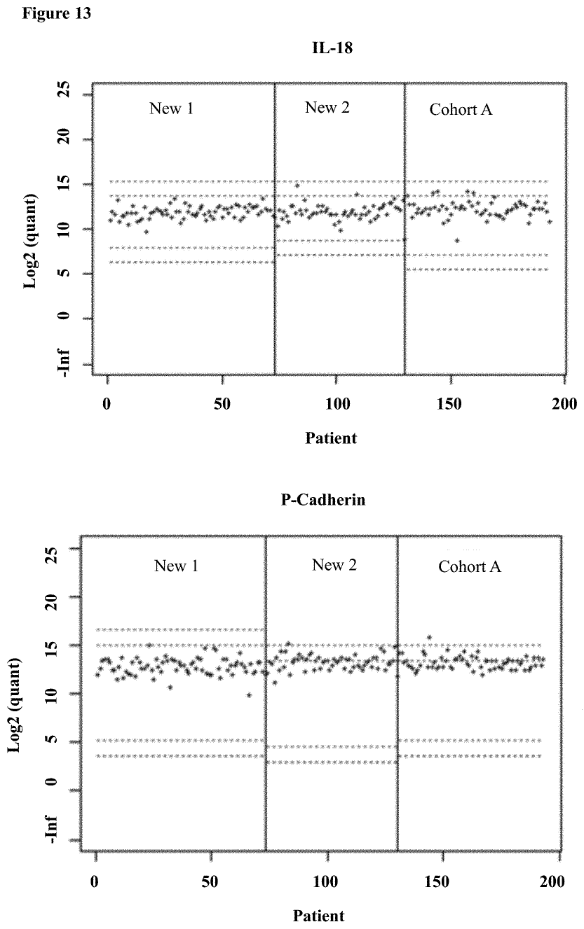

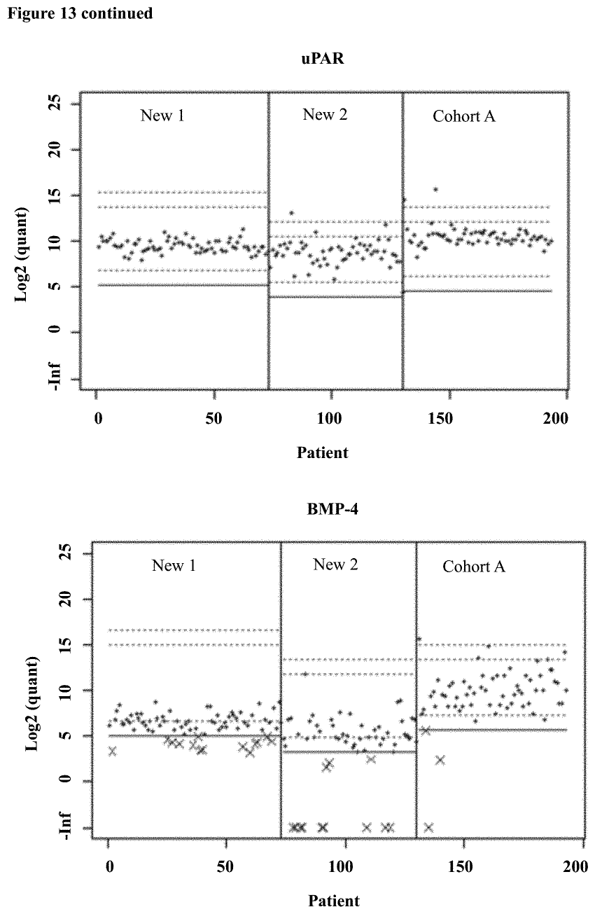

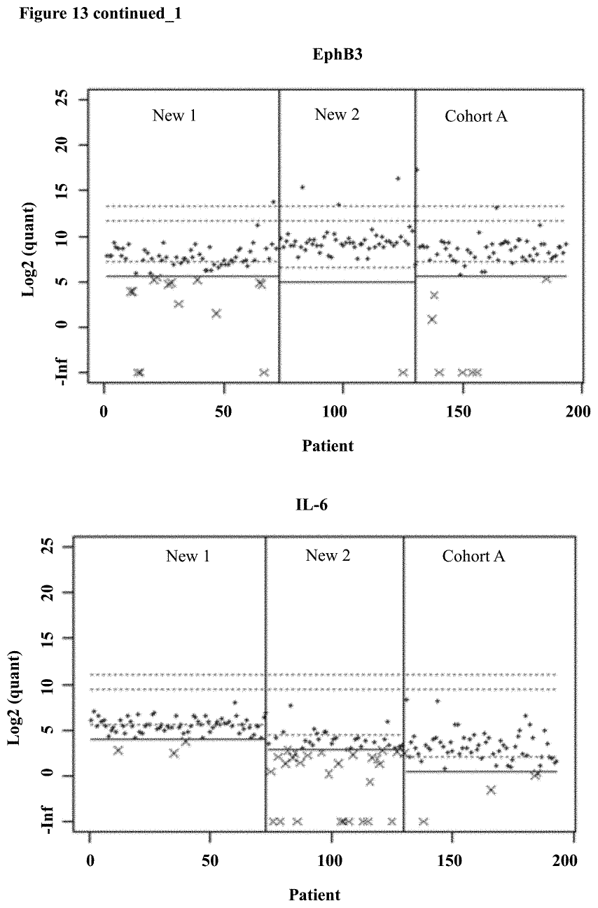

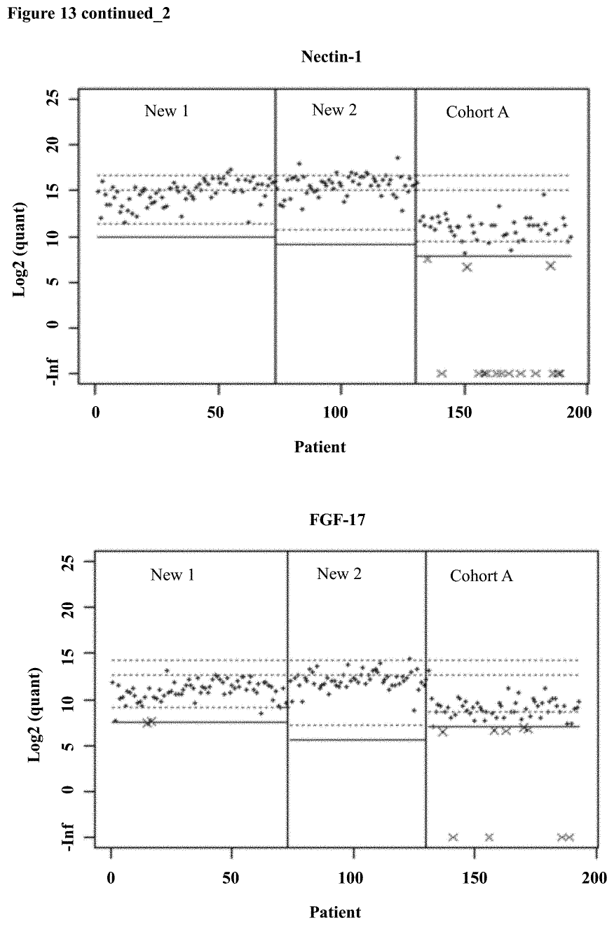

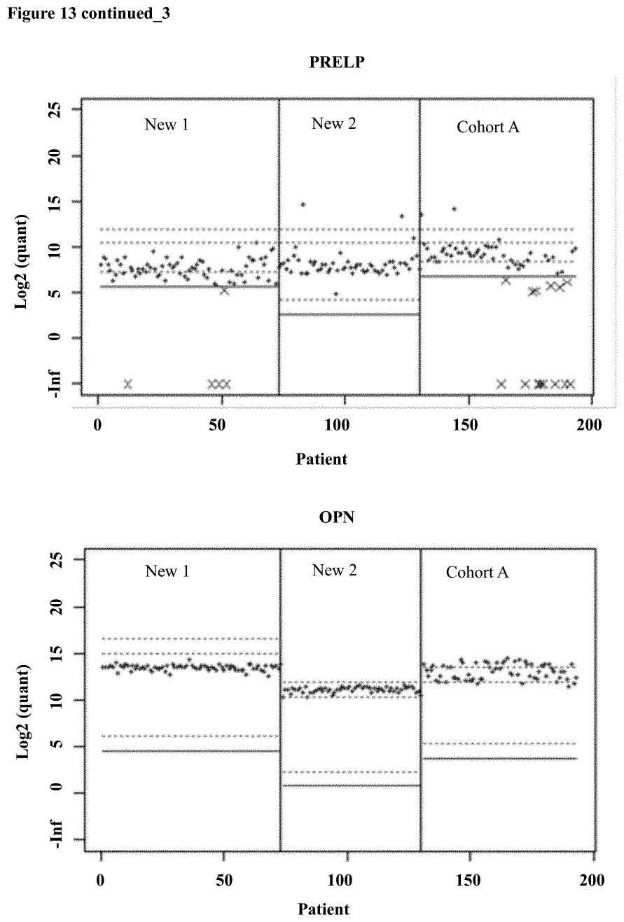

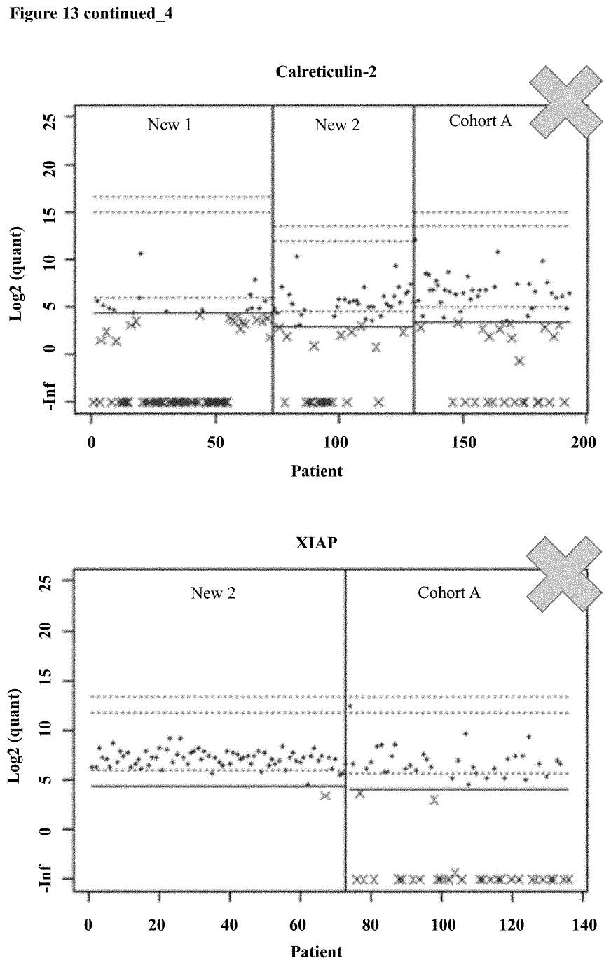

The most common proteins in the multi-protein models generated based on the cohort A dataset were examined for their measurability with similar values across all datasets (Cohort A and Cohort B New 1 and New 2 datasets). The measurability was very high for IL-18, uPAR and fairly high for P-Cadherin in all three datasets (FIG. 12) and the measured values were stable across the three datasets (FIG. 13). Calreticulin-2 and XIAP measurability values were low (many values were below the LOD), and thus they were excluded from any further analysis. Other proteins showed intermediate measurability and acceptable stability between datasets.

To validate the obtained protein signatures obtained using the Cohort A dataset on an independent dataset, Cohort B New 1 and Cohort B New 2 were combined into a single dataset. The dataset underwent the same analysis steps as the previous dataset: median values were subtracted for each protein. In order to prevent overfitting due to comparison of multiple models, the combined dataset was then randomly divided into two subsets: New 1+2 part 1 and New 1+2 part 2. Validation of all the previously obtained models was first performed on part 1. Then, only the three final models were validated over part 2 using ROC AUC with p-values, thus reducing bias of the measure due to overfitting.

The different protein signatures were examined without indicating the response annotation for any of the samples. The best performing SVM parameters found in the cohort A dataset were used in the validation procedure. First, simple models that were based on single proteins were examined (Table 7). Most proteins lost their predictive power in the cohort B part 1 dataset, probably due to the difference in patient cohort, tubes, batch effect as well as overfitting of the SEMMS output to the Cohort A dataset. Other proteins were not measured in the cohort B New datasets. However, P-Cadherin, Nectin-1, FGF-17, and IL-18 retained their predictive power.

TABLE 7
Validation of single-protein models on cohort B (part 1).

| | | Cohort B AUC |
|---|---|---|
| Proteins | Cohort A AUC | Part 1 |
| uPAR | 0.871 | 0.588 |
| P-Cadherin | 0.738 | 0.724 |
| IL-6 | 0.735 | 0.574 |
| Nectin-1 | 0.735 | 0.63 |
| VEGF-D | 0.721 | 0.515 |
| BMP-4 | 0.72 | 0.423 |
| PRELP | 0.702 | 0.495 |
| FGF-17 | 0.698 | 0.617 |
| MSP | 0.689 | 0.445 |
| EphB3 | 0.668 | 0.426 |
| OPN | 0.662 | 0.533 |
| IL-18 | 0.674 | 0.773 |
| Insulin R | 0.634 | NA |
| Neurturin | 0.633 | NA |
| Inhibin A | 0.632 | 0.44 |
| Cathepsin V | 0.632 | NA |
| MMP-7 | 0.605 | NA |
| RANK | 0.569 | NA |

Proteins that were not measured in Cohort B part 1 dataset or showed measurability below 50% are marked as NA.

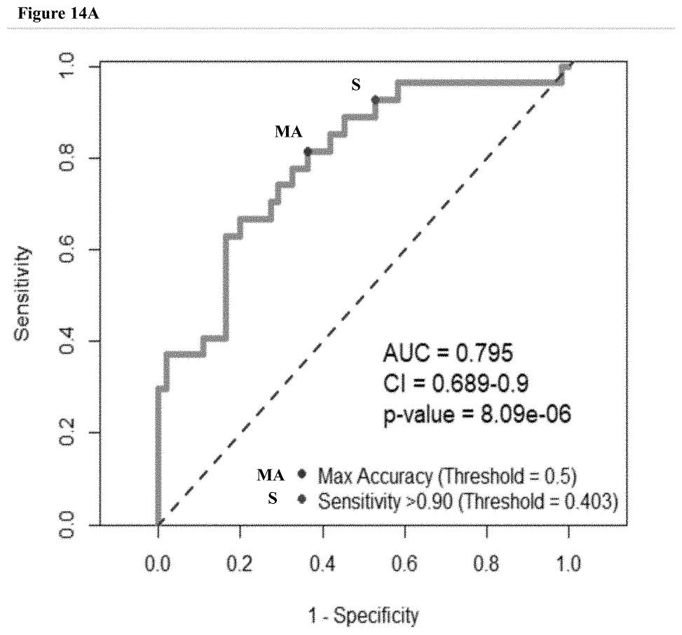

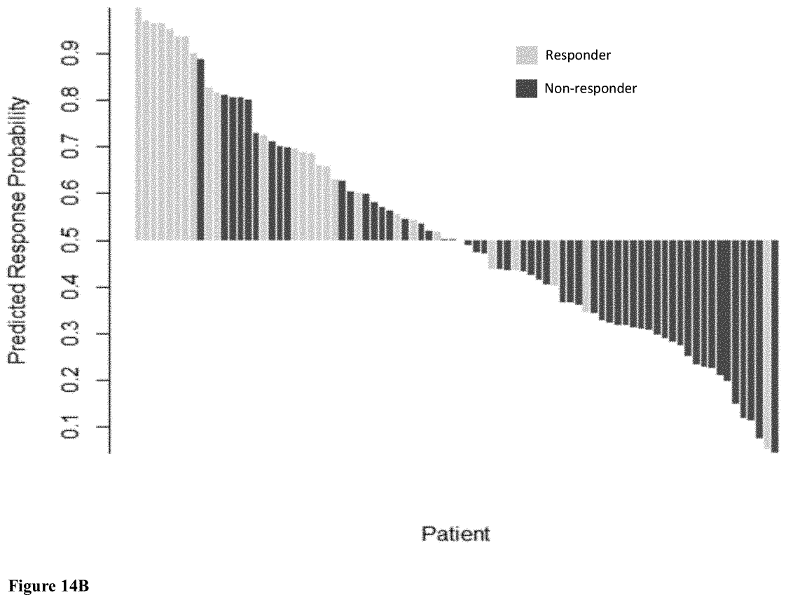

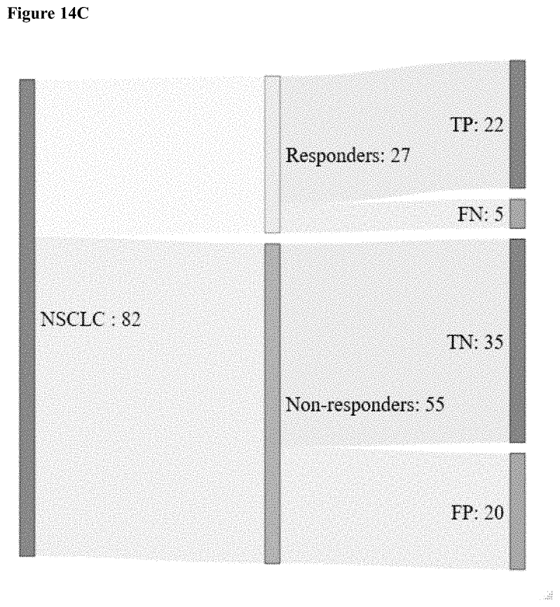

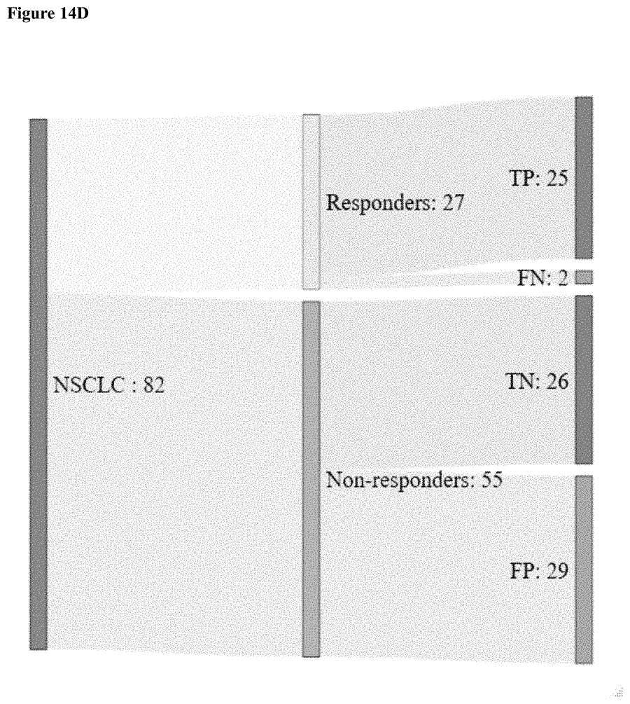

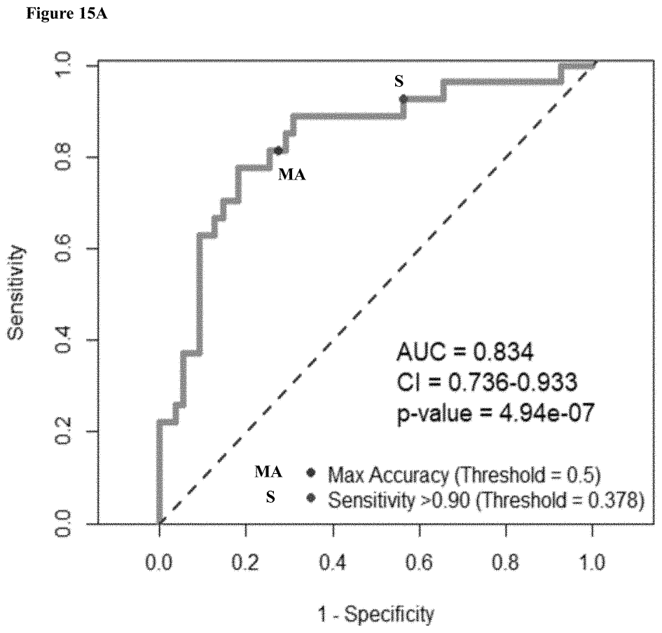

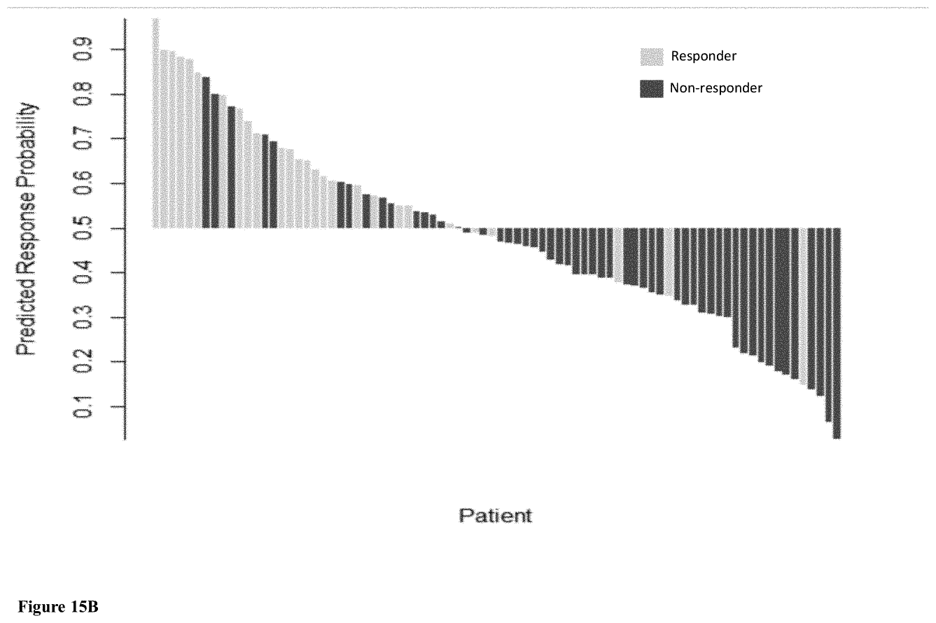

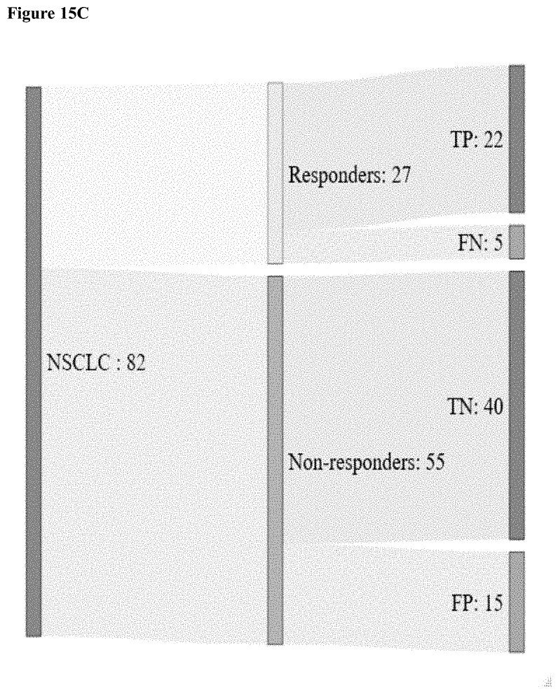

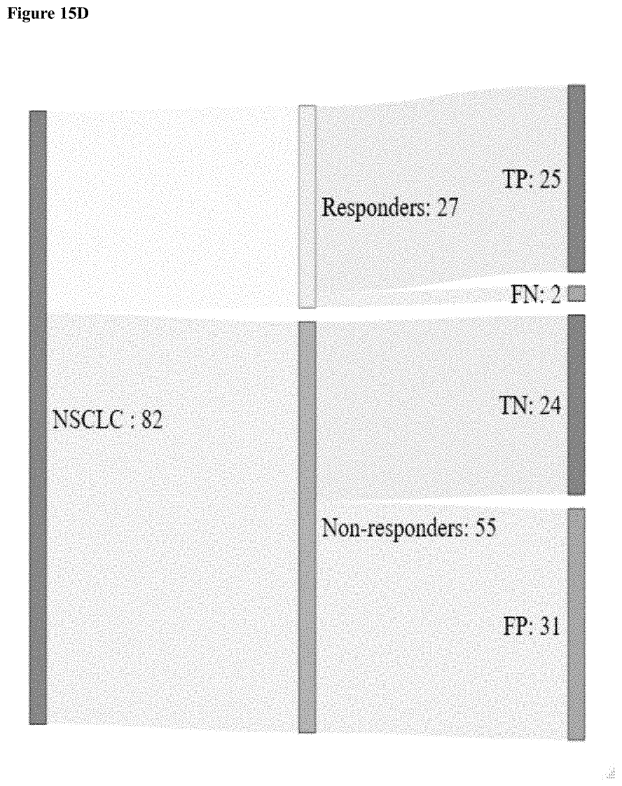

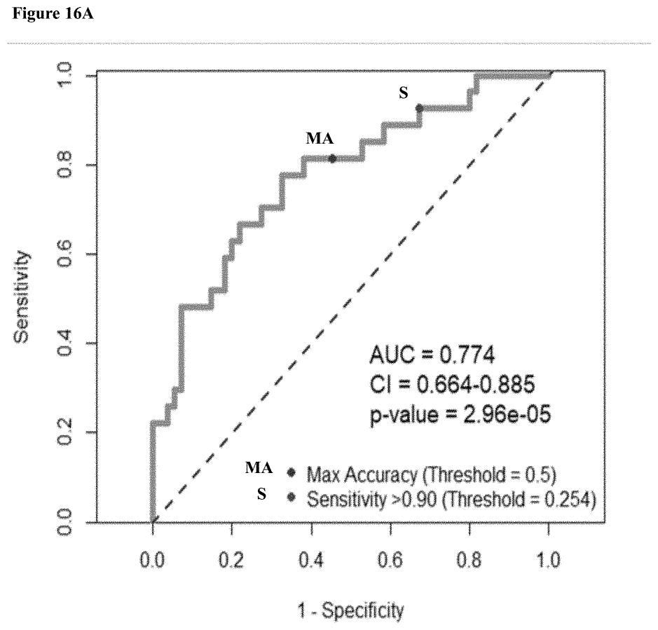

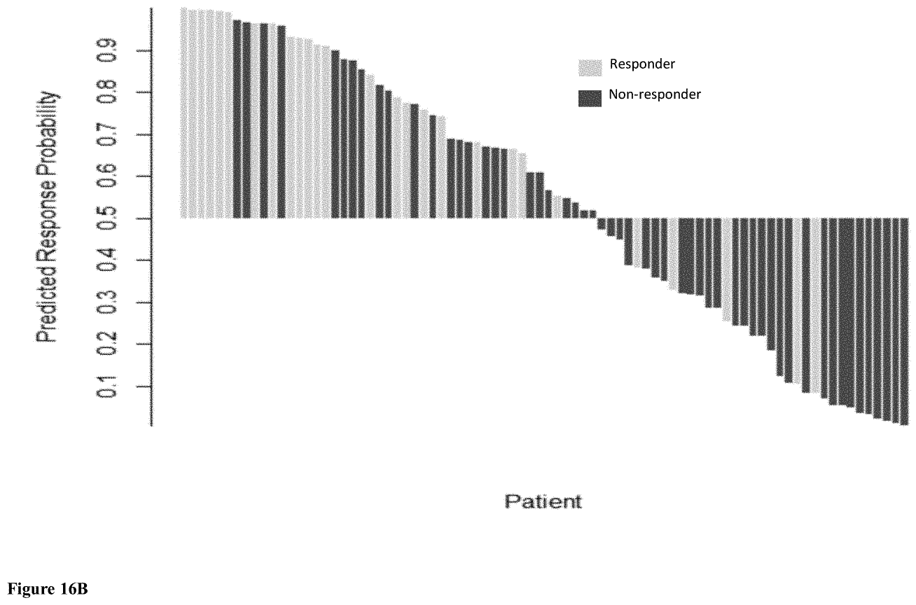

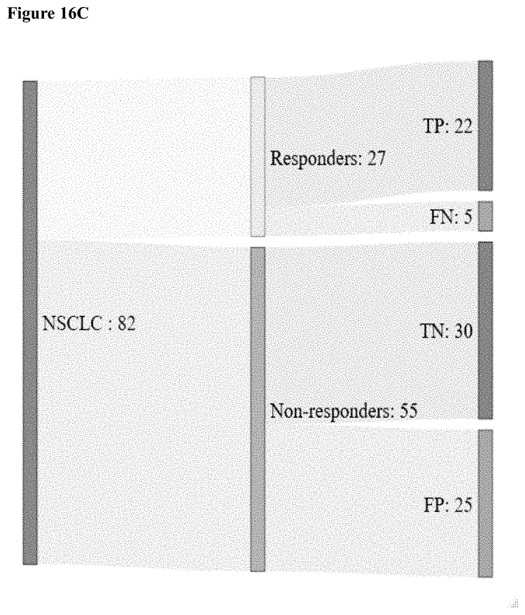

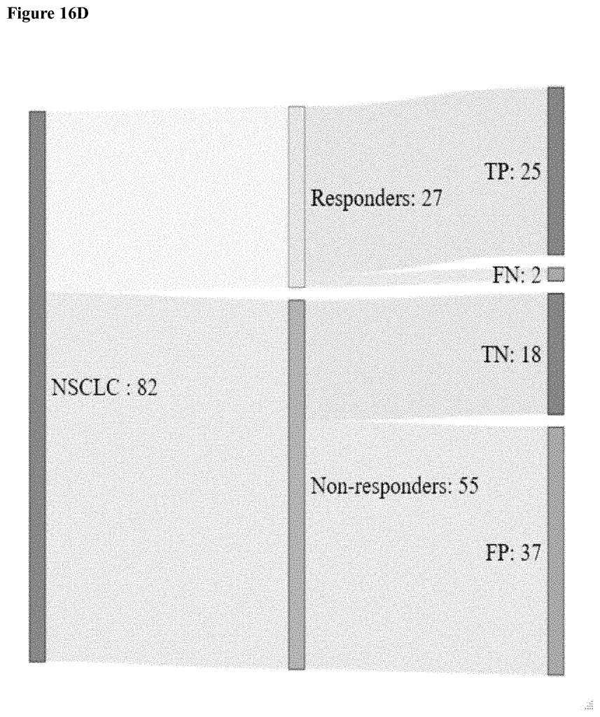

Because most single protein-based models did not generalize well to the New dataset or were not applicable since some predictive proteins were not measured in the New dataset, most multi-protein models also did not generalize well (Table 8). However, three models showed promising results on part 1 of the Cohort B dataset. Since multiple comparisons were performed on part 1, we used part 2 for testing the actual predictive value of these models (Table 9). The first model, based on P-Cadherin and Nectin-1, had a ROC AUC of 0.798 and p-value <0.001. The second model, that was based on P-Cadherin, IL-18, and FGF-17 had a ROC AUC of 0.775 and p-value <0.01. The third model, that was based on P-Cadherin and PRELP, had a ROC AUC of 0.835 and p-value <0.001. These results indicate that these models successfully generalized from the primary dataset to the New dataset. The overall results of applying these models on the full (part 1+2) New dataset are shown in FIG. 14, FIG. 15 and FIG. 16.

TABLE 8
Validation results of multi-protein models trained using the primary
dataset on part 1 of the New dataset. Models that contained proteins
that were either excluded during the batch QC process or simply not
measured in New dataset were rebuilt without these proteins.

| | | Cohort B |
|---|---|---|
| | Cohort A | Part 1 |
| Proteins | AUC | AUC |
| uPAR, P-Cadherin, IL-6, BMP-4, PRELP, OPN | 0.930 | 0.622 |
| uPAR, PRELP, IL-6 | 0.906 | 0.538 |
| uPAR, IL-6 | 0.905 | 0.492 |
| P-Cadherin, IL-6, BMP-4, PRELP, OPN | 0.897 | 0.681 |
| uPAR, PRELP | 0.891 | 0.531 |
| uPAR, IL-6, BMP-4 | 0.892 | 0.485 |
| P-Cadherin, PRELP | 0.858 | 0.712 |
| IL-6, Neurturin | 0.821 | NA |
| P-Cadherin, IL-18, FGF-17 | 0.817 | 0.913 |
| IL-6, Nectin-1 | 0.814 | 0.625 |
| P-Cadherin, Nectin-1 | 0.809 | 0.793 |
| PRELP, IL-6 | 0.800 | 0.538 |

TABLE 9
Validation results of the selected multi-protein models
on Cohort B part 2 and Cohort B full dataset.

| | Cohort A | Cohort B Validation AUC | | |
|---|---|---|---|---|
| Proteins | AUC | Part 1 | Part 2 | Full |
| P-Cadherin, | 0.809 | 0.793 | 0.798 (p < | 0.795 |
| Nectin-1 | | | 0.001) | |
| P-Cadherin, IL- | 0.817 | 0.913 | 0.775 (p < | 0.834 |
| 18, FGF-17 | | | 0.01) | |
| P-Cadherin, | 0.858 | 0.712 | 0.835 (p < | 0.774 |
| PRELP | | | 0.001) | |

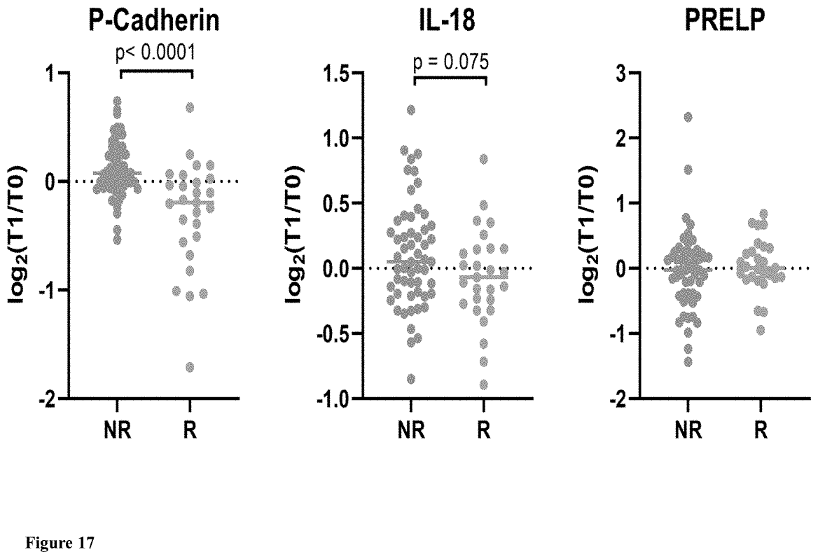

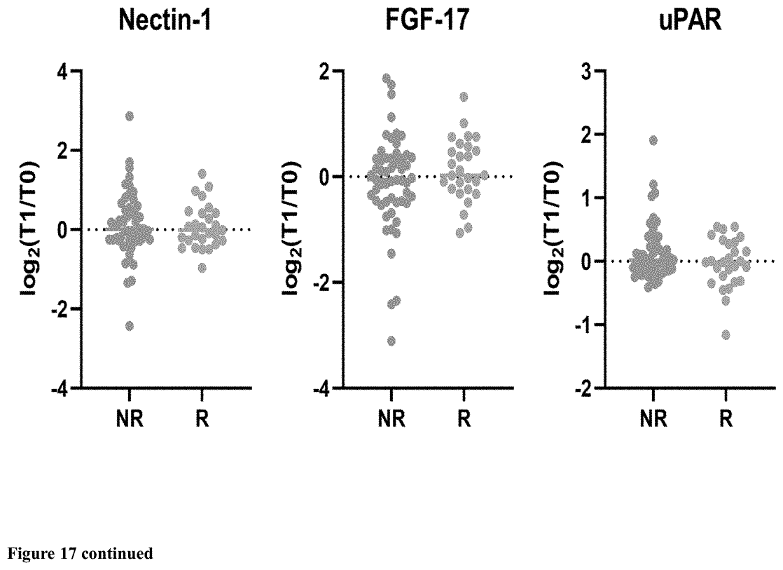

Examination of the expression levels of the signature proteins in the full validation set (parts 1+2) showed that P-Cadherin is the only protein that significantly changed. IL-18 also showed a trend towards significance. Although PRELP, Nectin-1 and FGF-17 helped in increasing predictivity, they showed similar levels between responders and non-responders (FIG. 17). Thus, the increased predictivity is likely to be due to statistical interaction with P-Cadherin expression levels. In contrast to the Cohort A dataset, uPAR levels in the Cohort B dataset is similar between responders and non-responders.

### Example 7: Cohort B Analysis

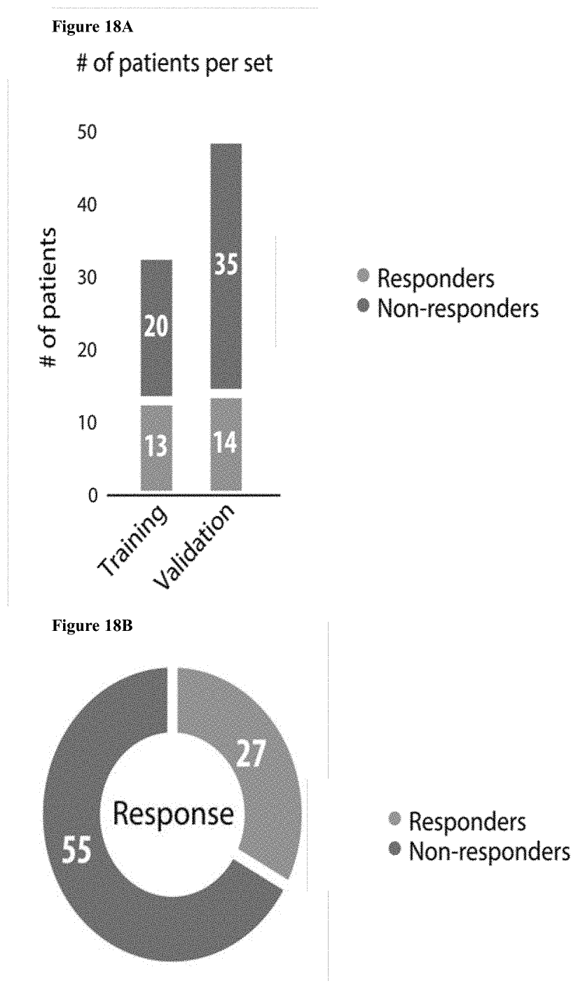

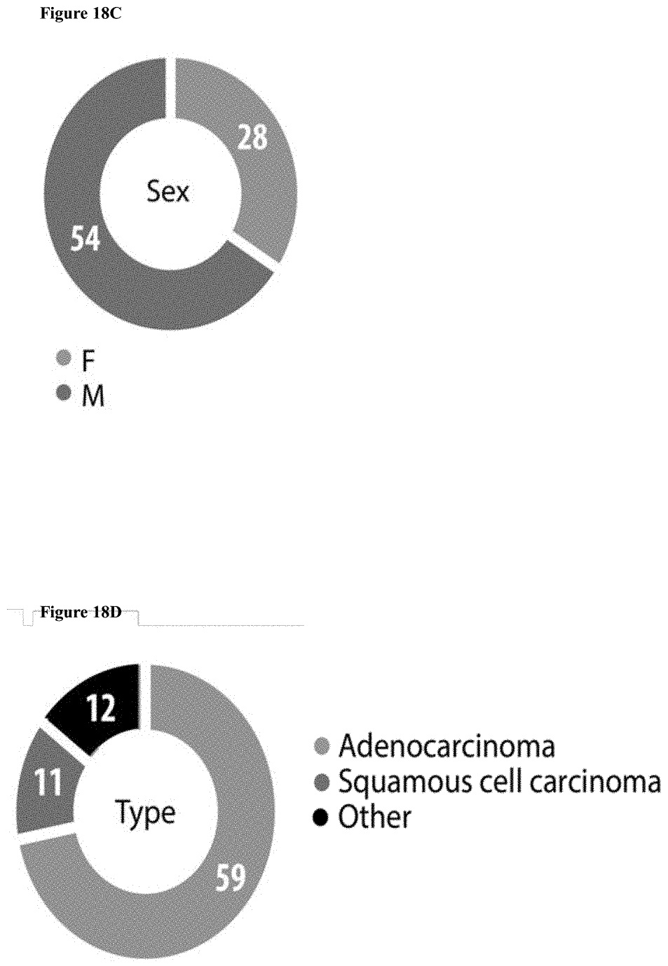

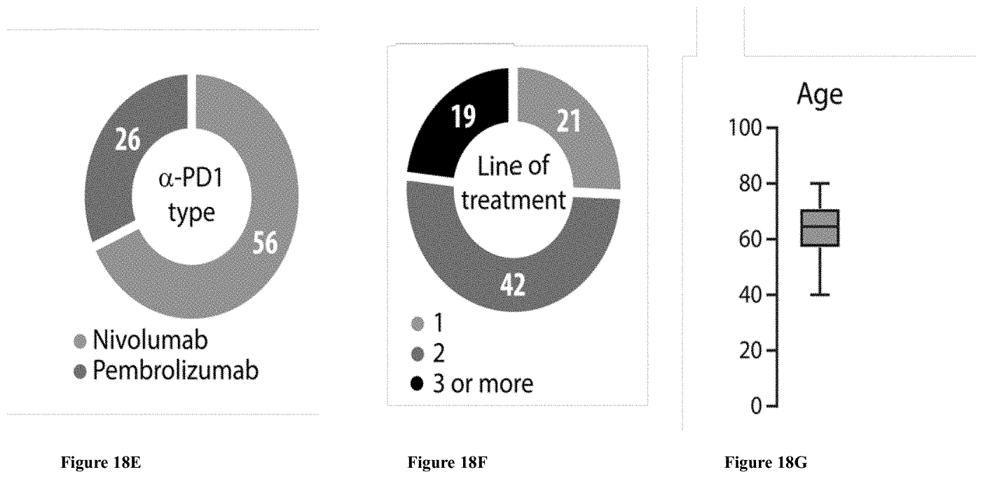

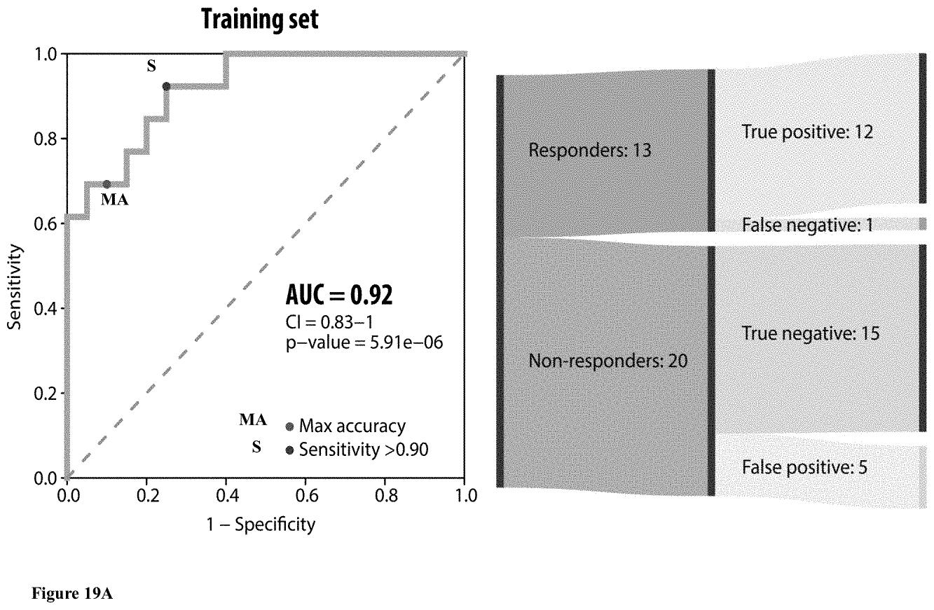

Next, cohort B comprising 82 advanced stage NSCLC patients mostly having adenocarcinoma treated with either pembrolizumab (approximately 70% of the patients) or nivolumab as a first- or second-line anti-PD-1 therapy was examined separately. As before, for each patient, plasma samples pre- (T0) and early on- (T1) treatment were collected, and the proteomic changes following anti-PD1 treatment was determined. The cohort was divided into training (n=33) and validation (n=49) sets (FIG. 18).

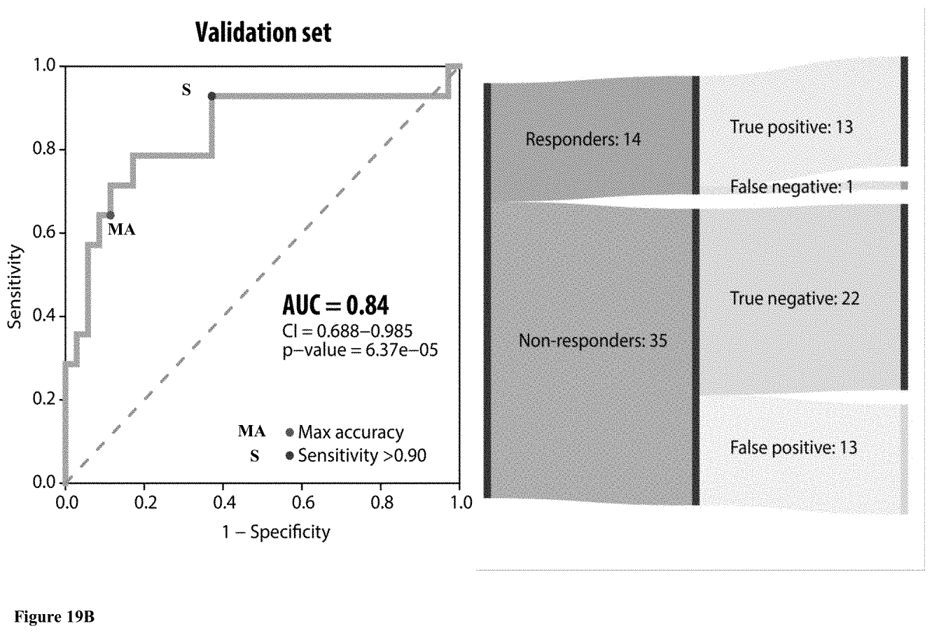

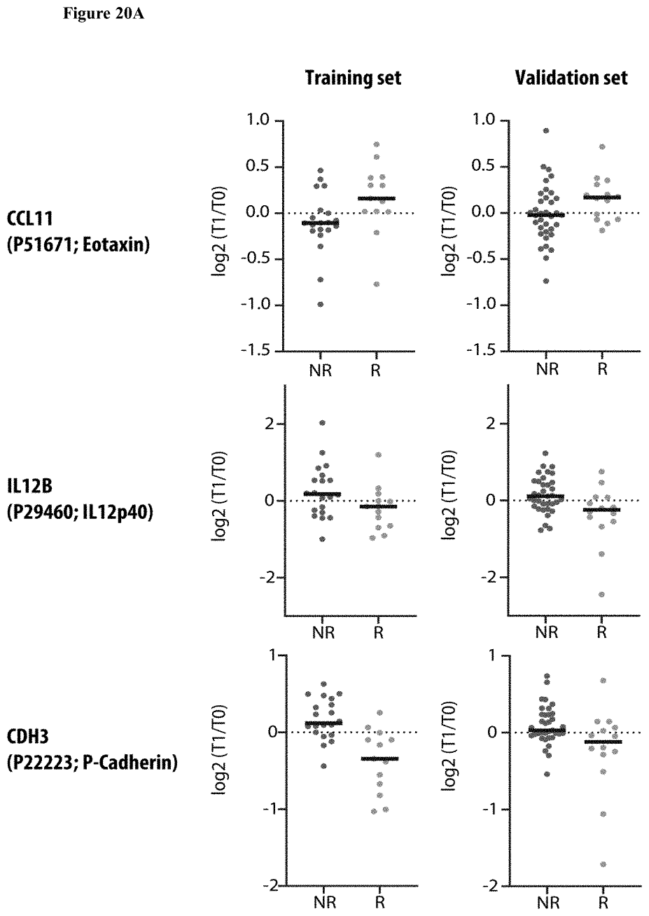

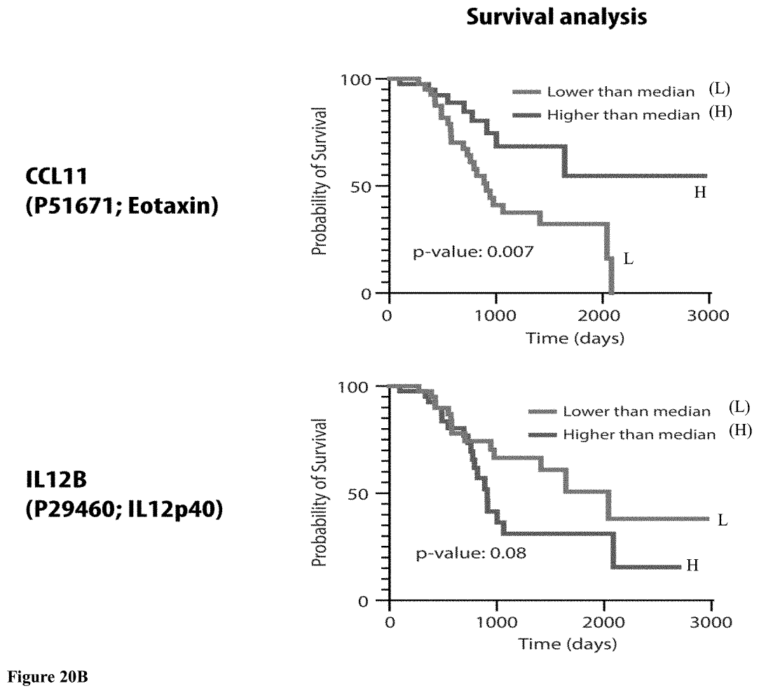

In order to identify predictive proteins for response, SVM algorithm was applied on the training set. Using this approach, a 3-protein signature composed of CCL11, IL12B and P-cadherin with high predictive power, as indicated by the area under the ROC curve of 0.92 (p-value 5.91E-06) (FIG. 19A). Further validation of the trained 3-protein signature obtained using the training dataset on the validation dataset demonstrated successful performance with AUC of 0.84 (6.37E-05) (FIG. 19B). As the aim was to have more than 90% sensitivity with maximal specificity, the confusion matrix resulted in only 1 false negative in each set (FIG. 19A-19B).

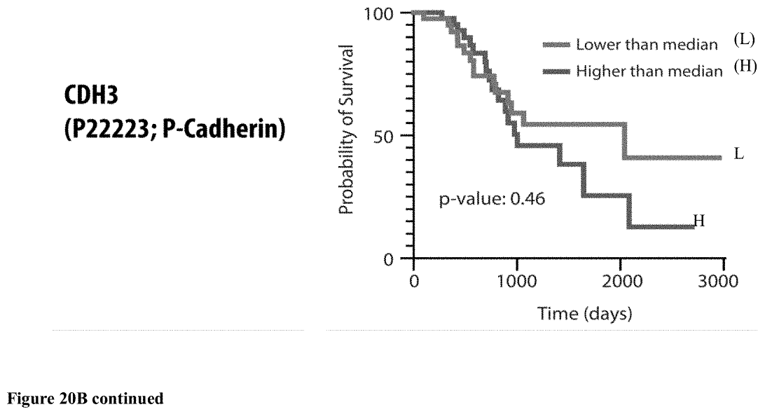

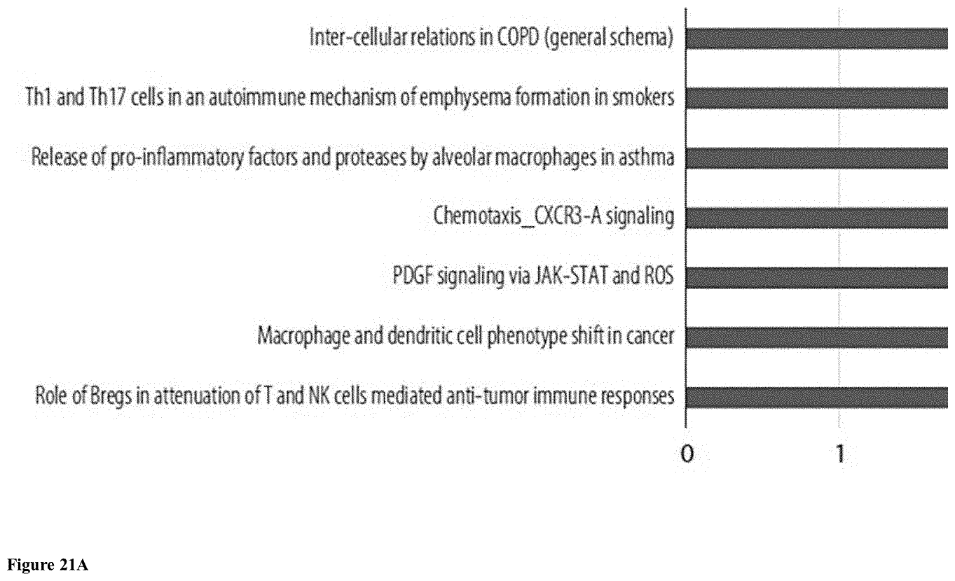

A deeper examination of the 3 proteins revealed that CCL11 was higher in the responder group, and indeed higher levels of this protein were significantly associated with increased overall survival (FIGS. 20A-20B); log-rank Kaplan Meier analysis; p-value=0.007). The other two proteins, IL12B and P-cadherin were higher in non-responders. These two proteins showed a trend of shorter survival when protein expression was high (FIGS. 20A-20B). Overall, these results indicate that the three proteins display an association between expression levels in the plasma and overall survival in accordance with the response prediction.

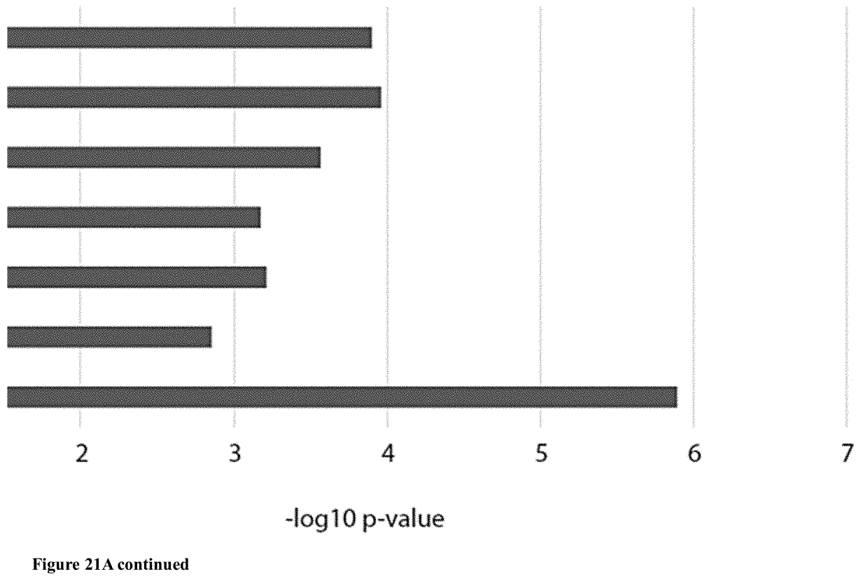

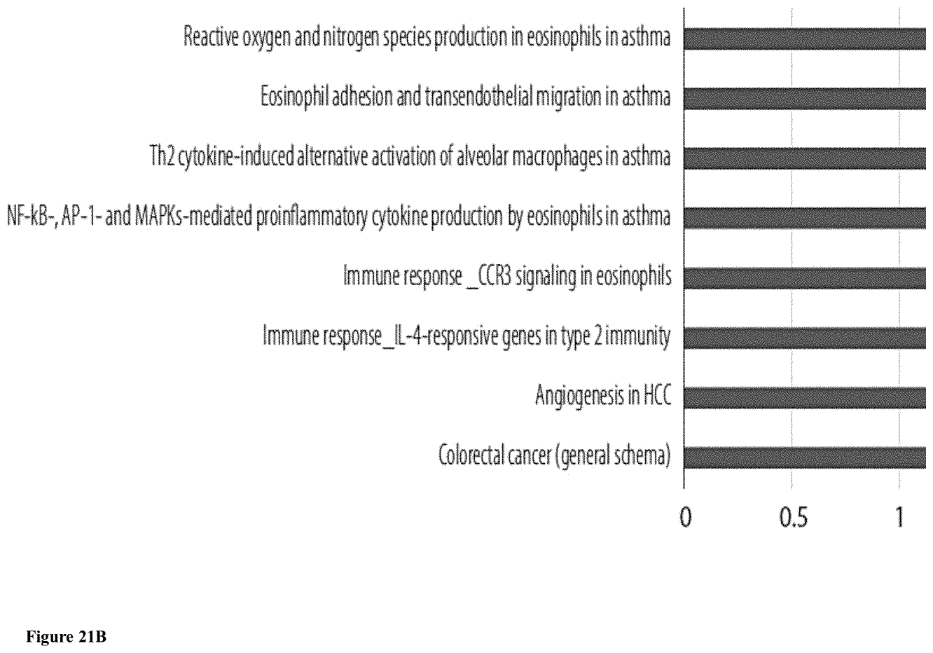

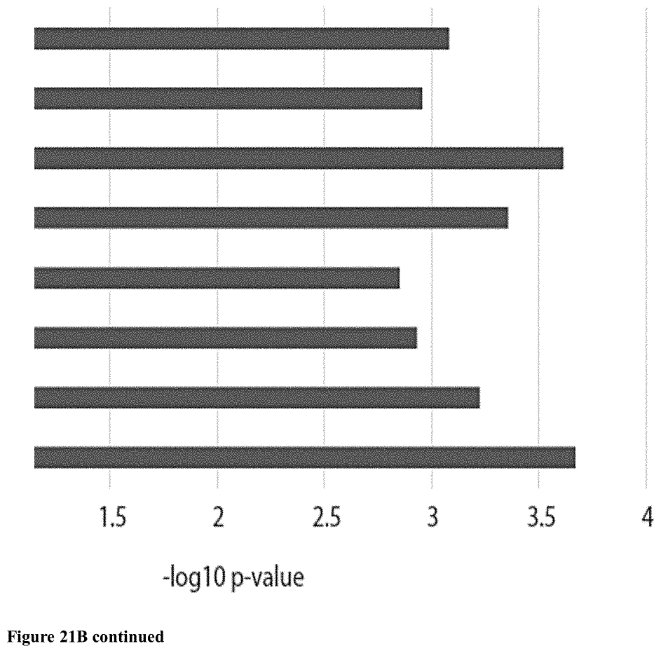

A further analysis of the biological functions that are associated with response revealed interesting differences between responders and non-responders (FDR p-value <0.05; analysis was done using Metacore). Importantly, non-responders had enrichment of immune-suppression related processes that involve regulatory B cells, macrophages and dendritic cell, which may contribute to resistance to therapy (FIG. 21A). Additionally, non-responders enriched biological processes included signaling pathways that may potentially be associated with resistance to treatment. Both responders and non-responders were enriched with pathways related to lung associated conditions, including asthma and chronic obstructive pulmonary disease (COPD; FIGS. 21A-21B).

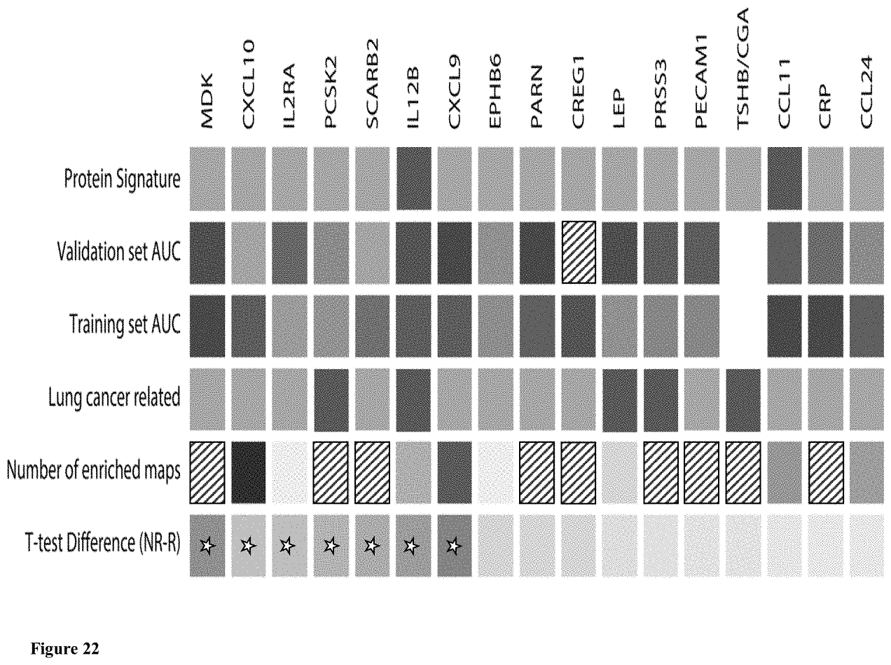

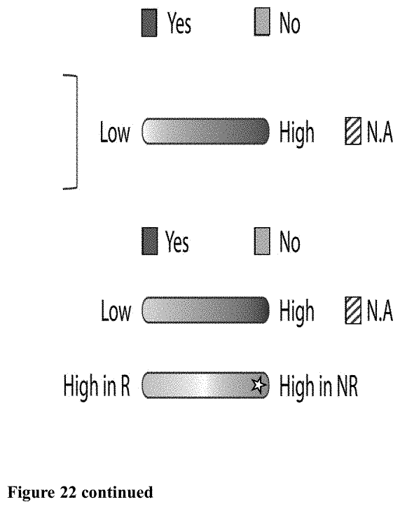

When integrating all the results into a single multi-layer analysis it is possible to identify potential targets for intervention, such as lung cancer-associated proteins that are both highly predictive and participate in many enriched biological processes (FIG. 22).

### Example 8: Confirmation with a Second Protein Array

Finally, the results achieved with the RayBiotech antibody array were compared with another antibody array (Olink). Of the proteins found to distinguish between responders and non-responders using the RayBiotech array, 11 were also found also to be differentially expressed when using the Olink array (AUC>0.60). The 11 significant proteins are provided in Table 10.

TABLE 10
Significant proteins based on both antibody arrays.

| RayBiotech Target | Olink Target | Uniprot | Olink Top Predictivity |
|---|---|---|---|
| CA125 | MUC-16 | Q8WXI7 | 0.68501 |
| IL-6 | IL6 | P05231 | 0.68475 |
| B7-H2 | ICOSLG | O75144 | 0.67655 |
| B4GalT1 | B4GALT1 | P15291 | 0.66056 |
| Cystatin B | CSTB | P04080 | 0.64098 |
| OPN | OPN | P10451 | 0.62943 |
| P-Cadherin | CDH3 | P22223 | 0.62939 |
| Nesfatin-1 | NUCB2 | P80303 | 0.62099 |
| PP | PPY | P01298 | 0.61737 |
| uPAR | U-PAR | Q03405 | 0.61046 |
| DFF45 | DFFA | O00273 | 0.60077 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for predicting a therapeutic response to immunotherapy in a subject suffering from lung cancer, the method comprising:
a) determining an expression level of at least two factors selected from Cadherin 3 (CDH3), Interleukin 18 (IL18), Plasminogen activator, urokinase receptor (PLAUR), Interleukin 6 (IL6), Nectin cell adhesion molecule 1 (NECTIN1), Vascular endothelial growth factor D (VEGFD), Bone morphogenetic protein 4 (BMP4), X-linked inhibitor of apoptosis (XIAP), Interleukin 2 (IL2), Proline and arginine rich end leucine rich repeat protein (PRELP), Fibroblast growth factor 17 (FGF17), Mucin 16, cell surface associated (MUC16), Periostin (POSTN), MST1, Keratin 18 (KRT18), Ephrin A4 (EFNA4), Interleukin 4 receptor (IL4R), Granulysin (GNLY), Beta-1,4-galactosyltransferase 1 (B4GALT1), Growth differentiation factor 2 (GDF2), Semaphorin 4D (SEMA4D), Erythropoietin receptor (EPOR), Ephrin type-B receptor 3 (EPHB3), Hepatocyte growth factor (HGF), Interferon alpha and beta receptor subunit 2 (IFNAR2), Secreted phosphoprotein 1 (SPP1), Fibronectin leucine rich transmembrane protein 1 (FLRT1), Inducible T cell costimulatory ligand (ICOSLG), Notch receptor 3 (NOTCH3), Neurturin (NRTN), Carbohydrate sulfotransferase 2 (CHST2), C-C motif chemokine ligand 1 (CCL1), Cluster of differentiation 97 (CD97), Linker for activation of T cells family member 2/non-T cell activation linker (LAT2), Dorsal inhibitory axon guidance protein (DRAXIN), Insulin like growth factor binding protein 4 (IGFBP4), TAFA Chemokine like family member 5 (TAFA5), Insulin like growth factor binding protein 5 (IGFBP-5), REST corepressor 1 (RCOR1), C-C motif chemokine ligand 11 (CCL11), Interleukin 12B (IL12B), Cystatin B (CSTB), Nucleobindin 2 (NUCB2), Pancreatic polypeptide (PPY) and DNA fragmentation factor subunit alpha (DFFA) in a biological sample obtained from said subject at a first time point relative to said immunotherapy;
b) determining an expression level of said at least two factors in a biological sample obtained from said subject at a second time point relative to said immunotherapy;
c) calculating a fold-change in expression of said at least two factors from said first time point to said second time point; and
d) analyzing said calculated fold-changes with a machine learning classifier, wherein said classifier is trained on a training set of fold-changes of said at least two factors in subjects who are known responders and non-responders, and wherein said classifier outputs a response prediction for said subject;
thereby predicting a therapeutic response to immunotherapy in a subject.

2. The method of claim 1, wherein a response prediction comprises a response score, and wherein a response score below a predetermined threshold indicates said subject is a non-responder, and a response score above a predetermined threshold indicates said subject is a responder.

3. The method of claim 1, wherein said first time point is a time point before said administration of said immunotherapy and said second time point is a time point after said administration of said immunotherapy.

4. A method for predicting a therapeutic response to immunotherapy in a subject suffering from lung cancer, the method comprising:
a) determining an expression level of at least one factor selected from CDH3 IL18 PLAUR, IL6, NECTIN1, VEGFD, BMP4, XIAP, IL2, PRELP, FGF17, MUC16, POSTN, MST1, KRT18, EFNA4, IL4R, GNLY, B4GALT1, GDF2, SEMA4D, EPOR, EPHB3, HGF, IFNAR2, SPP1, FLRT1, ICOSGL NOTCH3, NRTN, CHST2, CCL1, CD97, LAT2, DRAXIN, IGFBP4, TAFA5, IGFBP5, RCOR1, CCL11, IL12B, CSTB, NUCB2, PPY and DFFA in a biological sample obtained from said subject before initiation of said immunotherapy;
b) determining an expression level of said at least one factor in a biological sample obtained from said subject after initiation of immunotherapy; and
c) at least one of:
i. administering said immunotherapy between step (a) and step (b);
ii. continuing to administer said immunotherapy to a subject who is not a non-responder; and
iii. administering to a non-responder an agent that modulates a pathway differentially regulated in said non-responder.
wherein said factor is selected from BMP4, MUC16, KRT18, DRAXIN, EPHB3, EFNA4, GNLY, HGF, CCL1, IGFBP4, IL12B, IL18, IL2, IL4R, IL6, SPP1, CDH2, POSTN, SEMA4D, TAFA5, PLAUR and DFFA and an increased expression level of said at least one factor in said sample after initiation of immunotherapy is indicative of the subject being a non-responder to said immunotherapy, or wherein said factor is selected from B4GALT1, ICOSLG, BMP9, CCL11, CD97, CHST2, EPOR, FGF17, FLRT1, IFNAR2, IGFBP5, MST1, NECTIN1, NOTCH3, LAT2, NRTN, PRELP, RCOR1, VEGFD, CSTB, NUCB2, PPY and XIAP and an increased expression level of said at least one factor in said sample after initiation of immunotherapy is indicative of the subject being a responder to said immunotherapy, thereby predicting a therapeutic response to immunotherapy in a subject.

5. (canceled)

6. (canceled)

7. The method of claim 1, wherein determining an expression level comprises determining an expression level of a response signature, and wherein said response signature comprises at least a first factor selected from PLAUR, CDH3, and IL6, and at least a second factor selected from PLAUR, CDH3, IL6, BMP4, PRELP, SSP1, NRTN, NECTIN1, IL18, FGF17, CCL11, and IL12B.

8. The method of claim 1, wherein said immunotherapy is immune checkpoint blockade, optionally wherein said immune checkpoint blockade comprises an anti-PD-1/PD-L1 immunotherapy.

9. (canceled)

10. The method of claim 1, wherein said lung cancer is non-small cell lung cancer (NSCLC).

11. The method of claim 7, wherein said response signature is selected from the group consisting of:
a) CDH3, IL18 and FGF17;
b) PLAUR, CDH3, IL6, BMP4, PRELP and SPP1;
c) PLAUR, PRELP and IL6;
d) PLAUR and IL6;
e) CDH3, IL6, BMP4, PRELP and SPP1;
f) PLAUR and PRELP;
g) PLAUR, IL6, and BMP4;
h) CDH3 and PRELP;
i) IL6 and NRTN;
j) IL6 and NECTIN1;
k) CDH3 and NECTIN1;
l) PRELP and IL6; and
m) CDH3, CCL11 and IL12B.

12. The method of claim 1, wherein
a. said biological sample is plasma,
b. said expression level is a protein expression level;
c. said expression level is an mRNA expression level; or
d. a combination thereof.

13. (canceled)

14. The method of claim 12, wherein said expression level is a protein expression level.

15. The method of claim 1, comprising determining expression levels of a plurality of factors.

16. The method of claim 4, wherein said increased is by at least a pre-determined threshold.

17. (canceled)

18. (canceled)

19. (canceled)

20. A kit comprising reagents adapted to specifically determine the expression levels of at least two factors selected from CDH3, IL18, PLAUR, IL6, NECTIN1, VEGFD, BMP4, XIAP, IL2, PRELP, FGF17, MUC16, POSTN, MMST1SP, KRT18, EFNA4, IL4R, GNYL, B4GALT1, BMP9, SEMA4D, EPOR, EPHB3, HGF, IFNAR2, SPP1, FLRT1, ICOSLG, NOTCH3, CHST2, CCL1, CD97, LAT2, DRAXIN, IGFBP4, TAFA5, IGFBP5, RCOR1, NRTN, CCL11, IL12B, CSTB, NUCB2, PPY and DFFA and comprising at most 50 different reagents.

21. (canceled)

22. The kit of claim 20, comprising reagents adapted to specifically determine the expression level of at least a first factor selected from PLAUR, CDH3, and IL6, and at least a second factor selected from PLAUR, CDH3, IL6, BMP4, PRELP, SPP1, NRTN, NECTIN1, and IL18+FGF17.

23. The kit of claim 22, comprising reagents adapted to specifically determine the expression level of at least one group of factors selected from the group consisting of:
a) CDH3, IL18 and FGF17;
b) PLAUR, CDH3, IL6, BMP4, PRELP and SPP1;
c) PLAUR, PRELP and IL6;
d) PLAUR and IL6;
e) CDH3, IL6, BMP4, PRELP and SPP1;
f) PLAUR and PRELP;
g) PLAUR, IL6, and BMP4;
h) CDH3 and PRELP;
i) IL6 and NRTN;
j) IL6 and NECTIN1;
k) CDH3 and NECTIN1;
l) PRELP and IL6; and
m) CDH3, CCL11 and IL12B.

24. The kit of claim 20, wherein said expression level is selected from protein expression level and mRNA expression level.

25. The kit of claim 24, wherein said expression level is protein expression level and said reagents are antibodies.

26. The kit of claim 24, wherein said expression level is mRNA expression level and said reagents are isolated oligonucleotides, each oligonucleotide specifically hybridizing to a nucleic acid sequence of at least one of said factors.

27. The kit of claim 20, further comprising any one of: (i) a detectable tag or label, (ii) a secondary reagent for detection of said specific reagent, (iii) a solution for rendering a protein susceptible to binding or an mRNA susceptible to hybridization, (iv) a solution for lysing cells, (v) a solution for the purification of proteins or nucleic acids, (vi) any combination thereof.

28. The kit of claim 20, further comprising at least one reagent adapted to specifically determine the expression level of a control.
